SEQUENCE LISTING (51 sequences)

SEQ ID NO: 1 - protein, 4 aa, Artificial Sequence
  Description of Artificial Sequence Synthetic peptide
WMEW

SEQ ID NO: 2 - protein, 34 aa, Artificial Sequence
  Description of Artificial Sequence Synthetic peptide
WMEWDREINNYTSLIHSLIEESQNQQEKNEQELL

SEQ ID NO: 3 - protein, 35 aa, Artificial Sequence
  Description of Artificial Sequence Synthetic peptide
XWMEWDREINNYTSLIHSLIEESQNQQEKNEQELL

SEQ ID NO: 4 - protein, 35 aa, Artificial Sequence
  Description of Artificial Sequence Synthetic peptide
XWMEWDREINNYTSLIHSLIEESQNQQERNEQELL

SEQ ID NO: 5 - protein, 36 aa, Artificial Sequence
  Description of Artificial Sequence Synthetic peptide
XWMEWDREINNYTSLIHSLIEESQNQQEKNEQELLK

SEQ ID NO: 6 - protein, 36 aa, Artificial Sequence
  Description of Artificial Sequence Synthetic peptide
XWMEWDREINNYTSLIHSLIEESQNQQERNEQELLK

SEQ ID NO: 7 - protein, 36 aa, Artificial Sequence
  Description of Artificial Sequence Synthetic peptide
YTSLIHSLIEESQNQQEKNEQELLELDKWASLWNWF

SEQ ID NO: 8 - protein, 38 aa, Artificial Sequence
  Description of Artificial Sequence Synthetic peptide
NNLLRAIEAQQHLLQLTVWQIKQLQARILAVERYLKDQ

SEQ ID NO: 9 - protein, 34 aa, Artificial Sequence
  Description of Artificial Sequence Synthetic peptide
WMEWDREINNYTSLIHSLIEESQNQQERNEQELK

SEQ ID NO: 10 - protein, 34 aa, Artificial Sequence
  Description of Artificial Sequence Synthetic peptide
WMEWDREINNYTSLIHSLIEESQNQQERNEQEKL

SEQ ID NO: 11 - protein, 34 aa, Artificial Sequence
  Description of Artificial Sequence Synthetic peptide
WMEWDREINNYTSLIHSLIEESQNQQERNEQKLL

SEQ ID NO: 12 - protein, 34 aa, Artificial Sequence
  Description of Artificial Sequence Synthetic peptide
WMEWDREINNYTSLIHSLIEESQNQQERNEKELL

SEQ ID NO: 13 - protein, 34 aa, Artificial Sequence
  Description of Artificial Sequence Synthetic peptide
WMEWDREINNYTSLIHSLIEESQNQQERNKQELL

SEQ ID NO: 14 - protein, 34 aa, Artificial Sequence
  Description of Artificial Sequence Synthetic peptide
WMEWDREINNYTSLIHSLIEESQNQQERKEQELL

SEQ ID NO: 15 - protein, 35 aa, Artificial Sequence
  Description of Artificial Sequence Synthetic peptide
WMEWDREINNYTSLIHSLIEESQNQQERNEQELLK

SEQ ID NO: 16 - protein, 35 aa, Artificial Sequence
  Description of Artificial Sequence Synthetic peptide
WMEWDREINNYTSLIHSLIEESQNQQEKNEQELLK

SEQ ID NO: 17 - protein, 38 aa, Artificial Sequence; identical to SEQ ID NO: 8
  Description of Artificial Sequence Synthetic peptide

SEQ ID NO: 18 - protein, 36 aa, Artificial Sequence
  Description of Artificial Sequence Synthetic peptide
YTNTIYTLLEESQNQQEKNEQELLELDKWASLWNWF

SEQ ID NO: 19 - protein, 36 aa, Artificial Sequence
  Description of Artificial Sequence Synthetic peptide
YTGIIYNLLEESQNQQEKNEQELLELDKWANLWNWF

SEQ ID NO: 20 - protein, 36 aa, Artificial Sequence
  Description of Artificial Sequence Synthetic peptide
YTSLIYSLLEKSQIQQEKNEQELLELDKWASLWNWF

SEQ ID NO: 21 - protein, 33 aa, Artificial Sequence
  Description of Artificial Sequence Synthetic peptide
YTSVITIELSNIKENKCNGAKVKLIKQELDKYK

SEQ ID NO: 22 - protein, 33 aa, Artificial Sequence
  Description of Artificial Sequence Synthetic peptide
TSVITIELSNIKENKCNGAKVKLIKQELDKYKN

SEQ ID NO: 23 - protein, 33 aa, Artificial Sequence
  Description of Artificial Sequence Synthetic peptide
VITIELSNIKENKCNGAKVKLIKQELDKYKNAV

SEQ ID NO: 24 - protein, 33 aa, Artificial Sequence
  Description of Artificial Sequence Synthetic peptide
IALLSTNKAVVSLSNGVSVLTSKVLDLKNYIDK

SEQ ID NO: 25 - protein, 35 aa, Artificial Sequence
  Description of Artificial Sequence Synthetic peptide
VEAKQARSDIEKLKEAIRDTNKAVQSVQSSIGNLI

SEQ ID NO: 26 - protein, 35 aa, Artificial Sequence
  Description of Artificial Sequence Synthetic peptide
RSDIEKLKEAIRDTNKAVQSVQSSIGNLIVAIKSV

SEQ ID NO: 27 - protein, 35 aa, Artificial Sequence
  Description of Artificial Sequence Synthetic peptide
NSVALDPIDISIELNKAKSDLEESKEWIRRSNQKL

SEQ ID NO: 28 - protein, 35 aa, Artificial Sequence
  Description of Artificial Sequence Synthetic peptide
ALDPIDISIELNKAKSDLEESKEWIRRSNQKLDSI

SEQ ID NO: 29 - protein, 35 aa, Artificial Sequence
  Description of Artificial Sequence Synthetic peptide
LDPIDISIELNKAKSDLEESKEWIRRSNQKLDSIG

SEQ ID NO: 30 - protein, 35 aa, Artificial Sequence
  Description of Artificial Sequence Synthetic peptide
DPIDISIELNKAKSDLEESKEWIRRSNQKLDSIGN

SEQ ID NO: 31 - protein, 35 aa, Artificial Sequence
  Description of Artificial Sequence Synthetic peptide
PIDISIELNKAKSDLEESKEWIRRSNQKLDSIGNW

SEQ ID NO: 32 - protein, 35 aa, Artificial Sequence
  Description of Artificial Sequence Synthetic peptide
IDISIELNKAKSDLEESKEWIRRSNQKLDSIGNWH

SEQ ID NO: 33 - protein, 34 aa, Artificial Sequence
  Description of Artificial Sequence Synthetic peptide
HRIDLGPPISLERLDVGTNLGNAIAKLEAKELLE

SEQ ID NO: 34 - protein, 34 aa, Artificial Sequence
  Description of Artificial Sequence Synthetic peptide
IDLGPPISLERLDVGTNLGNAIAKLEAKELLESS

SEQ ID NO: 35 - protein, 34 aa, Artificial Sequence
  Description of Artificial Sequence Synthetic peptide
LGPPISLERLDVGTNLGNAIAKLEAKELLESSDQ

SEQ ID NO: 36 - protein, 34 aa, Artificial Sequence
  Description of Artificial Sequence Synthetic peptide
PISLERLDVGTNLGNAIAKLEAKELLESSDQILR

SEQ ID NO: 37 - protein, 35 aa, Artificial Sequence
  Description of Artificial Sequence Synthetic peptide
WQEWERKVDFLEENITALLEEAQIQQEKNMYELQK

SEQ ID NO: 38 - protein, 35 aa, Artificial Sequence
  Description of Artificial Sequence Synthetic peptide
QEWERKVDFLEENITALLEEAQIQQEKNMYELQKL

SEQ ID NO: 39 - protein, 35 aa, Artificial Sequence
  Description of Artificial Sequence Synthetic peptide
EWERKVDFLEENITALLEEAQIQQEKNMYELQKLN

SEQ ID NO: 40 - protein, 35 aa, Artificial Sequence
  Description of Artificial Sequence Synthetic peptide
WERKVDFLEENITALLEEAQIQQEKNMYELQKLNS

SEQ ID NO: 41 - protein, 35 aa, Artificial Sequence
  Description of Artificial Sequence Synthetic peptide
ERKVDFLEENITALLEEAQIQQEKNMYELQKLNSW

SEQ ID NO: 42 - protein, 35 aa, Artificial Sequence
  Description of Artificial Sequence Synthetic peptide
RKVDFLEENITALLEEAQIQQEKNMYELQKLNSWD

SEQ ID NO: 43 - protein, 35 aa, Artificial Sequence
  Description of Artificial Sequence Synthetic peptide
KVDFLEENITALLEEAQIQQEKNMYELQKLNSWDV

SEQ ID NO: 44 - protein, 35 aa, Artificial Sequence
  Description of Artificial Sequence Synthetic peptide
VDFLEENITALLEEAQIQQEKNMYELQKLNSWDVF

SEQ ID NO: 45 - protein, 35 aa, Artificial Sequence
  Description of Artificial Sequence Synthetic peptide
DFLEENITALLEEAQIQQEKNMYELQKLNSWDVFG

SEQ ID NO: 46 - protein, 35 aa, Artificial Sequence
  Description of Artificial Sequence Synthetic peptide
FLEENITALLEEAQIQQEKNMYELQKLNSWDVFGN

SEQ ID NO: 47 - protein, 8 aa, Artificial Sequence
  Description of Artificial Sequence Synthetic peptide
WASLWNWF

SEQ ID NO: 48 - protein, 34 aa, Artificial Sequence
  Description of Artificial Sequence Synthetic peptide
WMEWDREINNYTKLIHSLIEESQNQQEKNEQELL

SEQ ID NO: 49 - protein, 34 aa, Artificial Sequence
  Description of Artificial Sequence Synthetic peptide
WMEWDREINKYTSLIHSLIEESQNQQEKNEQELL

SEQ ID NO: 50 - protein, 34 aa, Artificial Sequence
  Description of Artificial Sequence Synthetic peptide
WMEWDREIKNYTSLIHSLIEESQNQQEKNEQELL

SEQ ID NO: 51 - protein, 37 aa, Artificial Sequence
  Description of Artificial Sequence Synthetic peptide
YTSLIHSLIEESQNQQEKNEQELLELDKWASLWNWFK

(19) United States
(12) Patent Application Publication
(10) Pub. No.: US 2009/0088377 A1
(43) Pub. Date: Apr. 2, 2009

(54) CYSTEIC ACID DERIVATIVES OF ANTI-VIRAL PEPTIDES

(75) Inventors: Omar Quraishi, Westmount (CA); Martin Robitaille, St. Columban (CA); Dominique P. Bridon, Franklin, MA (US)

Correspondence Address: LOWRIE, LANDO & ANASTASI, LLP, ONE MAIN STREET, SUITE 1100, CAMBRIDGE, MA, 02142, US

(73) Assignee: CONJUCHEM BIOTECHNOLOGIES, INC., Montreal (CA)

(21) Appl. No.: 12/122,627

(22) Filed: May 16, 2008

Related U.S. Application Data
(60) Provisional application No. 60/938,380, filed on May 16, 2007.
(60) Provisional application No. 60/938,394, filed on May 16, 2007.

(51) Int. Cl. A61K 38/16; C07K 14/00; C07K 14/76

(52) U.S. Cl. 514/12; 530/324; 530/362

(57) ABSTRACT

This invention relates to C34 peptide derivatives having improved aqueous solubility that are inhibitors of viral infection and/or exhibit antifusogenic properties. In particular, this invention relates to C34 derivatives having inhibiting activity against human immunodeficiency virus (HIV), respiratory synctial virus (RSV), human parainfluenza virus (HPV), measles virus (MeV), and simian immunodeficiency virus (SIV) with long duration of action for the treatment of the respective viral infections.

### CROSS REFERENCE TO RELATED APPLICATIONS

This application claims priority to U.S. Ser. No. 60/938,380 and U.S. Ser. No. 60/938,394, both of which were filed on May 16, 2007. The contents of the aforementioned applications are hereby incorporated by reference in their entirety.

### BACKGROUND OF THE INVENTION

Entry of human immunodeficiency virus type 1 (HIV-1) into uninfected cells encompasses three main steps: (i) the binding of gp120 to the CD4 receptor, (ii) the subsequent binding to co-receptor CXCR4 or CCR5, and (iii) a series of conformational changes of the ectodomain of the HIV-1 transmembrane glycoprotein gp41 that are important to trigger membrane fusion events that ultimately permit the infection to occur. Viruses such as respiratory syncytial virus (RSV), human parainfluenza virus type 3 (HPIV-3), measles virus and simian immunodeficiency virus (SIV) show a high degree of structural and functional similarity with HIV, including a gp41-like protein.

Several small molecule drug candidates, including those that inhibit binding to CD4 or to the CCR5 co-receptor, are either in human clinical trials or are close to market approval (Meanwell N A, Kadow J F (2003) Curr Opinion Drug Disc & Develop 6: 451-461; Olson W C, Maddon P J (2003) Curr Drug Targets-Infectious Disord 3: 283-294). Several synthetic peptides are known that inhibit or otherwise disrupt membrane fusion-associated events, including, for example, inhibiting retroviral transmission to uninfected cells. For example, the synthetic peptides C34, T1249, DP-107 and T-20 (DP-178), which are derived from separate domains within gp41, are potent inhibitors of HIV-1 infection and HIV induced cell-cell fusion.

T-20 (DP-178, enfuvirtide, Fuzeon®, Trimeris/Roche Applied Sciences) is a synthetic peptide based on the CHR sequence of HIV-1 gp41, and is believed to target the conformational rearrangements of gp41. It had been widely believed that T-20 inhibition was due to its ability to bind to the hydrophobic grooves of the NHR region of gp41 resulting in the inhibition of six-helix bundle formation (Kliger Y, Shai Y (2000) J Mol Biol 295: 163-168). Contrary to this view, recent studies have suggested that T-20 is capable of targeting multiple sites in gp41 and gp120 (Liu S et al. (2005) J Biol Chem 280:11259-11273). For example, T-20 binds and oligomerizes at the surface of membranes, thereby inhibiting recruitment and oligomerization of gp41 at the plasma membrane of infected cells (Muñoz-Barroso I et al. (1998) J Cell Biol 140: 315-23; Kliger Y et al. (2001) J Biol Chem 276:1391-1397). Furthermore, it has also been shown that the ectodomain of gp41 within a region immediately adjacent to the membrane-spanning domain having the peptide sequence, ⁶⁶⁶WASLWNWF⁶⁷³, constitutes a higher affinity site for T-20 than the NHR of gp41 (Munoz-Barroso I et al. (1998) supra 140: 315-23; Kliger Y et al. (2001) supra).

Another C-peptide, C34, composed of a peptide sequence which overlaps with T-20 but contains the gp41 coiled-coil cavity binding residues, ⁶²⁸WMEW⁶³¹, is known to compete with the CHR of gp41 for the hydrophobic grooves of the NHR region (Liu S et al. (2005) J Biol Chem 280:11259-11273).

While many of the anti-viral or anti-fusogenic peptides described in the art exhibit potent anti-viral and/or anti-fusogenic activity, these peptides suffer from poor solubility in aqueous formulations at physiological pH, as well as short plasma half-lifes in vivo. There is therefore a need for a method of increasing the solubility and prolonging the half-life of existing anti-viral and/or anti-fusogenic peptides, thus providing for water soluble, longer acting anti-viral and/or anti-fusogenic peptides in vivo.

### SUMMARY OF THE INVENTION

The present invention is directed to, at least in part, modified anti-viral and/or anti-fusogenic peptides having increased solubility in aqueous solution at physiological pH, compared to the peptides prior to modification. In one embodiment, the peptides of the invention are modified to include one or more polar groups or moieties, e.g., one or more cysteic acids, thereby increasing their solubilities in aqueous solutions. The modified peptides can further include chemically reactive moieties such that the modified peptides can react with available functionalities on blood components or carrier proteins, e.g., albumin (e.g., human serum albumin or recombinant albumin), thus increasing the stability in vivo of the modified peptides. In embodiments, the modified peptides are conjugated to the blood components or carrier proteins, e.g., albumin (e.g., human serum albumin, recombinant albumin, or other carrier proteins). These modified peptides, or conjugates thereof, thereby reduce, e.g., the need for more frequent, or even continual, administration of the peptides. The modified peptides of the present invention can be used, e.g., prophylactically and/or therapeutically for ameliorating infection of a number of viruses, including human immunodeficiency virus (HIV), human respiratory syncytial virus (RSV), human parainfluenza virus (HPIV), measles virus (MeV) and simian immunodeficiency virus (SIV). Modification of other peptides involved in viral transfection (e.g., Hepatitis, Epstein Barr and other related viruses) is also within the scope of the invention.

Accordingly, in one aspect the invention features a modified anti-viral and/or anti-fusogenic peptide having increased solubility in aqueous or water solution at a pH ranging from about 5 to 8 (e.g., at physiological pH), compared to the peptide prior to modification. In one embodiment, the modified anti-viral and/or anti-fusogenic peptide remains substantially soluble (e.g., less than about 40%, 30%, 20% 10% precipitation in water or aqueous solution at a pH ranging from about 5 to 8 (e.g., at physiological pH)) in a concentrated solution (e.g., a concentration in the range of about 10 to 500 mg/ml, about 10 to 400 mg/ml, about 10 to 300 mg/ml, about 10 to 200 mg/ml, about 10 to 180 mg/ml, about 40 to 180 mg/ml, about 60 to 180 mg/ml, or about 90 to 100 mg/ml, in aqueous solution (e.g., an isotonic or high salt aqueous solution). In embodiments, the modified anti-viral and/or anti-fusogenic peptide shows a solubility limit (i.e., the maximal concentration to maintain a clear solution) that is at least about 1.3, 1.5, 1.8, 2, 2.3, 2.5, 2.8, 3 or 3.5-fold higher than the peptide prior to modification. In embodiments, the modified anti-viral and/or anti-fusogenic peptide has a solubility limit of at least about 20 mg/ml, 25 mg/ml, 30 mg/ml, 35 mg/ml or 40 mg/ml in aqueous, isotonic solution at a pH ranging from about 5 to 8. An “aqueous solution” as used herein includes, without limitation, water, saline solution (e.g., isotonic solutions), buffers made in water (e.g., sodium phosphate buffer), aqueous gels, and aqueous formulations at a pH suitable for administration to a subject (e.g., a human subject), e.g., subcutaneous, intravenous pulmonary, intramuscular or intraperitoneal administration; or a formulation at a pH suitable for a manufacturing process.

In embodiments, the modified anti-viral and/or anti-fusogenic peptide includes one or more polar moieties. In one embodiment, the modified anti-viral and/or anti-fusogenic includes one or more polar moieties that are either charged or uncharged at physiological pH. In some embodiments, the side chains may be neutral and can increase the overall solubility of the modified peptide in an aqueous solution through, e.g., hydrogen bonding or other non-covalent interactions. For example, in certain instances a neutral side chain with oxygen or nitrogen groups is capable of hydrogen bonding to bulk solvent and may be used to increase the overall solubility of the peptide. In some embodiments, the side chain may be any non-natural polar or neutral side chain, e.g., a side chain not found in the twenty naturally occurring amino acids.

In embodiments, the polar moiety of the modified anti-viral and/or anti-fusogenic peptide includes the following structure:

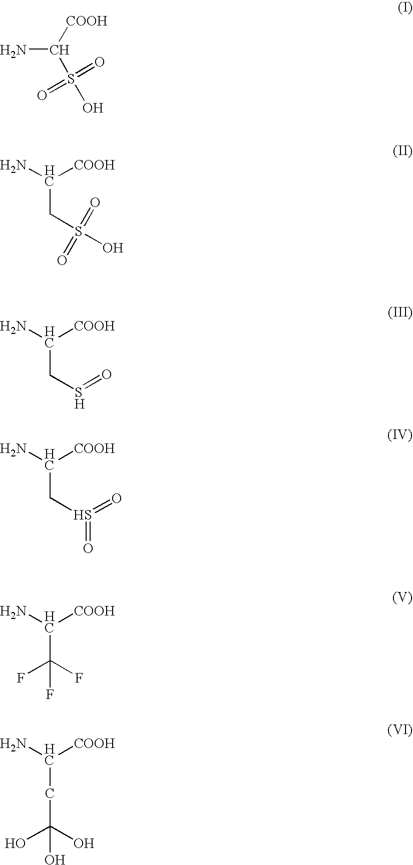

For example, the modified anti-viral and/or anti-fusogenic peptide can include one or more cysteic acids. In embodiments, the cysteic acid has the structure:

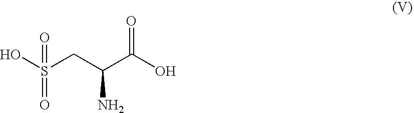

Additional suitable side chains that can increase the solubility of the peptides disclosed herein will be readily selected by the person of ordinary skill in the art, given the benefit of this disclosure.

In other embodiments, the one or more polar moieties (e.g., cysteic acids) are added to the N-terminal or C-terminal end of the anti-viral and/or anti-fusogenic peptide. In other embodiments, the one or more polar moieties are added to the internal sequence of the anti-viral and/or anti-fusogenic peptide.

In one embodiment, the modified anti-viral and/or anti-fusogenic peptide includes at least a portion of a gp41 coiled-coil cavity binding residues. For example, the peptide can include residues ⁶²⁸WMEW⁶³¹ (SEQ ID NO:1), or the amino acid sequence having up to one amino acid substitution (e.g., conservative or non-conservative substitution) or addition thereto. In other embodiments, the anti-viral and/or anti-fusogenic peptide includes the full or partial native amino acid sequence of C34 from amino acids ⁶²⁸WMEWDREINNYTSLIHSLIEESQNQQEKNEQELL⁶⁶¹ (corresponding to amino acids C1 to C34) (SEQ ID NO:2), or up to five, four, three, two or one amino acid substitutions (e.g., conservative or non-conservative substitution), deletions, or additions thereto.

In other embodiments, the modified anti-viral and/or anti-fusogenic peptide includes the amino acid sequence of DP107 and DP178 peptides and analogs thereof, including peptides comprised of amino acid sequences from other (non-HIV) viruses that correspond to the gp41 region of HIV from which DP107 and DP178 are derived and that exhibit anti-viral and/or anti-fusogenic activity. More particularly, these peptides can exhibit anti-viral activity against, among others, human respiratory syncytial virus (RSV), human parainfluenza virus (HPV), measles virus (MeV) and simian immunodeficiency virus (SIV). The invention also relates to modified peptides of SEQ ID NO: 1 to SEQ ID NO:86 of US 05/0070475, specifically incorporated by reference herein.

In embodiments, the modified anti-viral and/or anti-fusogenic peptides of the invention further include one or more chemically reactive moieties or groups such that the modified peptides can react with available functionalities on blood components or carrier proteins to form stable covalent bonds, thereby producing conjugated peptide forms. In one embodiment, the modified peptide comprises one or more reactive groups which react with one or more amino groups, hydroxyl groups, or thiol groups on one or more blood components (e.g., albumin) to form stable covalent bonds. For example, the peptide-reactive group albumin conjugates can be about a 1:1 molar ratio of peptide to albumin. Typically, the conjugation occurs via a covalent bond between the reactive group and amino acid 34 (Cys³⁴) of albumin, e.g., human albumin.

In another embodiment, the reactive group can be a maleimide-containing group (e.g., MPA (maleimido propionic acid) or GMBA (gamma-maleimide-butyralamide)) which is reactive with a thiol group on a blood protein, including a mobile blood protein such as albumin. The reactive modification or group can further include one or more linkers. In embodiments, the linker is chosen from one or more of: (2-amino)ethoxy acetic acid (ABA), [2-(2-amino)ethoxy)]ethoxy acetic acid (AEEA), ethylenediamine (EDA); one or more alkyl chains (C1-C10) such as 8-aminooctanoic acid (AOA), 8-aminopropanoic acid (APA), or 4-aminobenzoic acid (APhA). The reactive group, with or without linker, can be added to the N- or C-terminal of the anti-viral and/or anti-fusogenic modified peptide, typically, the C-terminal of the anti-viral and/or anti-fusogenic modified peptide. In other embodiments, the reactive group is attached to an internal residue of the modified peptide (e.g., attached to an epsilon NH₂ group of an internal lysine residue; a hydroxyl group of an internal serine residue (e.g., Serine 13 of C34)). Non-limiting examples of C34 modified peptides are disclosed in WO 02/096935, the entire contents of which are incorporated by reference herein in their entirety.

Typically, when one or more polar moieties (e.g., cysteic acids) are added to one end (e.g., the N-terminal end) of the modified anti-viral and/or anti-fusogenic peptide, the reactive group is added to the opposite end (e.g., the C-terminal end). For example, the modified peptides can have one of the following configurations:

[Polar Moiety (e.g., cysteic acid)−MODIFIED PEPTIDE−Linkerₙ−Reactive Group] (VI); or

[Reactive Group−Linkerₙ−MODIFIED PEPTIDE−Polar Moiety (e.g., cysteic acid)] (VII).

wherein the reactive group can be, e.g., a maleimide-containing group, with or without a linker, e.g., n can be 0, 1, 2, 3, 4 or more linkers. When more than one linker are present, the linkers may be the same, e.g., AEEA-AEEA, or different, e.g., AEEA-EDA or AEA-AEEA.

In certain embodiments, the additional group for inclusion in the modified anti-viral and/or anti-fusogenic peptide may be a compound having formula (I).

(R₁)ₘ-X-(R₂)ₙ (VIII)

In formula (VIII), the sum of m and n is at least 1 and m and n are each integers that are zero or greater. For example, where m is zero, then n is 1 or greater, and where n is zero, then m is 1 or greater. X is an anti-viral and/or anti-fusogenic peptide, such as, for example, C34, T20, T1249 or an analog or derivative thereof including, for example, maleimide derivative thereof. Where R₁ is present and R₂ is absent, R₁ is present at the N-terminus of the X group. When R₁ is absent and R₂ is present, R₂ is present at the C-terminus of the X group.

In certain examples, R₁ and R₂ may each be independently selected from a compound having formula (IX).

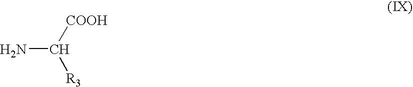

The core structure of formula (IX) is similar to that of an amino acid and includes an amino group, an alpha carbon and a carboxyl group. Depending on the exact position of the R₁ and R₂ groups in the peptide derivative, the groups may be bound to the peptide through different atoms of formula (IX). For example, where R₁ is a compound having formula (IX), R₁ may be bound to the peptide through the carboxyl group of formula (IX) to provide a peptide bond between the carboxyl group of R₁ and an amino group of the peptide. Where R₂ is a compound having formula (IX), R₂ may be bound to the peptide through the amino group of formula (IX) to provide a peptide bond between the amino group of R₂ and a carboxy group of the peptide.

In some embodiments, the R₃ group of formula (IX) may be any polar, uncharged group other than the polar, uncharged groups commonly found in the 20 naturally occurring amino acids. For example, the R₃ group may be, or may include, a sulfonyl group (HS═(O)₂), a sulfoxide group (HS═O), a sulfonic acid group (HO—S═(O)₂), a haloalkyl group, a secondary amine, a tertiary amine, a hydroxyl group, or other side chain group that is polar or even neutral and that can increase the overall solubility of the peptide derivative in an aqueous solution. For example, a side chain with groups capable of hydrogen bonding may be used to increase the overall solubility of the peptide. In certain examples, the side chain is preferably non-reactive such that unwanted side reactions with a linker or other species do not occur to any substantial degree. In some examples, the above-noted groups for R₃ may be spaced from the alpha carbon, for example, by 1-3 carbon atoms. In certain examples, R₃ may be selected to provide a compound having formulae (X) —(XV).

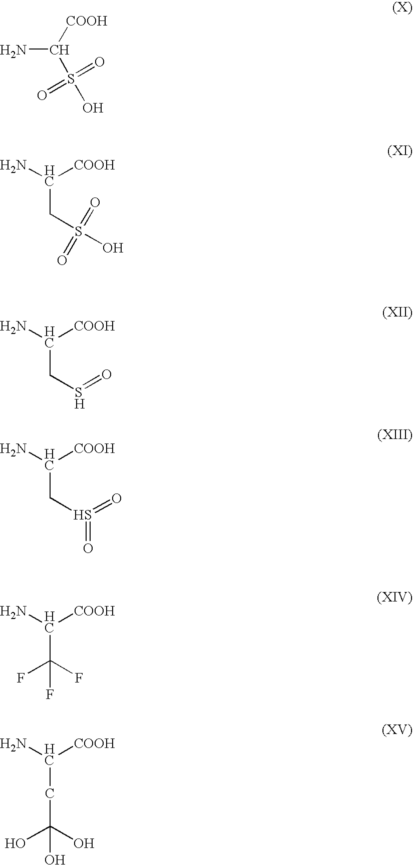

Additional suitable side chains that can increase the solubility of the peptides disclosed herein will be readily selected by the person of ordinary skill in the art, given the benefit of this disclosure.

In certain embodiments, the R₁ and R₂ groups do not substantially affect the overall secondary, or in certain instances the tertiary structure, of the peptide conjugate. By not substantially affecting the secondary structure of the peptide conjugate, the overall activity of the peptide conjugate should not be appreciably less than that of the non-derivatized peptide.

In other embodiments, the peptide derivative may take the form of a composition as shown in formula (XVI).

X₁-(R₁)ₘ-X₂-(R2)ₙ (XVI)

In formula (XVI), X₁ and X₂ represent portions of a peptide that when joined together would provide, for example, C34, T20, or T1249, or a variant thereof. In formula (XVI), R₁ and R₂ may be any of those groups discussed above in reference to formula (IX), and the sum of m and n is an integer greater than or equal to 1, with the possibility that either m or n may be zero. In formula (XVI), the group has been inserted into the middle of the peptide chain. Such insertion may be performed using many different methods including enzymatic digestion of the peptide, followed by insertion of an R₁ or R₂ group or both and then subsequent attachment of the peptide fragments together.

In certain embodiments, the compounds disclosed herein may be linked to one or more additional groups at the N-terminus, the C-terminus or through a side chain of one or more of the amino acids of the peptide. For example, compositions as shown schematically in formulae (XVII)-(XX) may be produced.

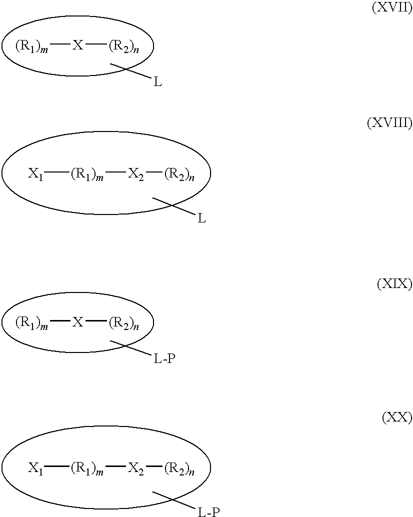

In formulas (XVII)-(XX), L is a linker such as, for example, (2-amino)ethoxy acetic acid (AEA), ethylenediamine (EDA), 2-[2-(2-amino)ethoxy]ethoxy acetic acid (AEAA), alkyl chain motifs (C1-C10) such as glycine, 3-aminopropionic acid (APA), 8-aminooctoanic acid (AOA), 4-aminobenzoic acid (APhA) or the like, and R₁ and R₂ may be any of those groups discussed herein. The linker may be bound to the peptide through any amino acid of the peptide, for example, through an amino group of a lysine, a thiol group, a hydroxyl group in one or more amino acid side chain residues of the peptide; or at the N-terminus or at the C-terminus of the peptide. The X, X₁ and X₂ groups are a peptide (X) or peptide fragments (X₁ and X₂). The P group shown in formulae (XIX) and (XX) represents a protein that may be conjugated to the derivatized peptide through the linker L. Illustrative proteins include a blood protein or a carrier protein (e.g., human serum albumin, recombinant albumin, an immunoglobulin or fragment thereof, a transferrin or other suitable proteins.

The protein conjugates (formulae (XIX) and (XX)) may be produced ex vivo or in vivo. Where in vivo production occurs, compounds, such as those shown in formulae (XVII) and (XVIII), may be introduced into a subject and react with an in vivo protein such as albumin.

Anti-viral and/or anti-fusogenic peptides of the invention can have one or more amino acid substitutions or additions. For example, the peptides can have one or more conservative or non-conservative substitutions. In certain embodiments, the modified peptides can further include one or more amino acid residues. For example, the modified peptides of C34 can optionally have a substitution of native Lysine at position 28 (Lys²⁸) for an arginine and/or add a Lys residue (or a Lysine residue modified at its 1-nitrogen atom to be covalently coupled directly or indirectly to a reactive group as described herein (e.g., AEEA-MPA) at the C-terminal end. It should be understood that within group Lys (ε-AEEA-MPA), AEEA-MPA is attached to the epsilon NH₂ group of lysine.

Non-limiting examples of modified anti-viral and/or anti-fusogenic modified peptides of C34 of the present invention include the following sequences:

CA Compound I: (Cysteic Acid (CA) directly linked to C34; also referred to herein as CA-C34 (SEQ ID NO:3).

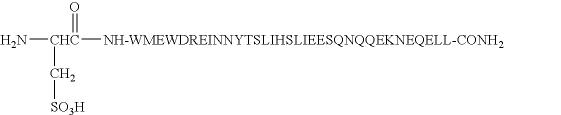

CA Compound II: (Cysteic Acid (CA) directly linked to C34 having a substitution of native Lysine at position 28 (Lys²⁸) for an arginine; also referred to herein as CA-C34 (Arg²⁸) (SEQ ID NO:4).

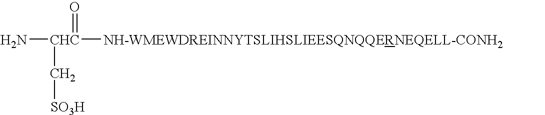

CA Compound III: (Cysteic Acid (CA) directly linked to C34 having an additional Lysine residue at position 35 (Lys³⁵), wherein the epsilon NH₂ group of lysine is coupled to the reactive group via linker (AEEA-MPA); also referred to herein as CA-C34-Lys³⁵ (ε-AEEA-MPA) (SEQ ID NO:5).

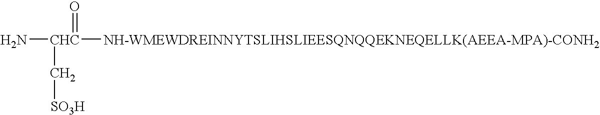

and
CA Compound IV: (Cysteic Acid (CA) directly linked to C34 having a substitution of native Lysine at position 28 (Lys²⁸) for an arginine; an additional Lysine residue at position 35 (Lys³⁵), wherein the epsilon NH₂ group of lysine is coupled to the reactive group via linker (AEEA-MPA); also referred to herein as CA-C34 (Arg²⁸)-Lys³⁵ (E-AEEA-MPA) (SEQ ID NO:6).

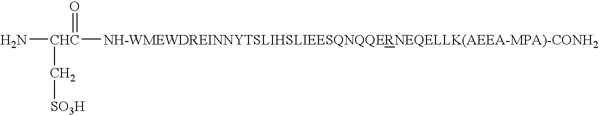

In yet another aspect, the invention features conjugates of the modified anti-viral and/or anti-fusogenic peptides described herein having one or more chemically reactive modifications coupled to available functionalities on one or more blood components. In one embodiment of the invention, the modified peptides comprise a reactive group which is coupled to amino groups, hydroxyl groups, or thiol groups on blood components to form stable covalent bonds. The maleimide group can be directly coupled to the modified peptide or can be coupled indirectly, e.g., via a linker (e.g., a linker as described herein). In another embodiment of the invention, the reactive group can be a maleimide which is reactive with a thiol group on a blood protein, including a mobile blood protein such as albumin. The peptide-reactive group albumin conjugates can be about a 1:1 molar ratio of peptide to albumin. Typically, the conjugation occurs via a covalent bond between the reactive group and amino acid 34 (Cys³⁴) of human albumin.

The modified anti-viral and/or anti-fusogenic peptide can include a reactive moiety, e.g., a maleimide-containing group, that has the ability to covalently bond one or more blood components, e.g., serum albumin, so as to form a conjugate. The conjugation step can occur in vivo, e.g., after administraton of the modified peptide to a subject. Alternatively, the conjugation step can occur ex vivo or in vitro, e.g., by contacting the modified peptide containing the reactive group with a blood components, e.g., albumin. The preparation and uses of conjugates of C34, DP107, DP178 and the like are disclosed in WO 02/096935 and US 05/0070475, incorporated by reference herein in their entirety. The conjugates formed in vivo or ex vivo are useful in inhibiting the viral and/or fusogenic activity of viruses, such as HIV, RSV, HPV, MeV or SIV in a subject, e.g., a human subject.

In another aspect, the invention features, compositions, e.g., pharmaceutical compositions, that include one or modified anti-viral and/or anti-fusogenic peptides as described herein, and a pharmaceutically acceptable carrier. In embodiments, the compostions are suitable for injection (e.g., subcutaneous or intravascular injection), as well as pulmonary, intramuscular and/or intraperitoneal delivery. In other embodiments, the compositions are suitable for manufacturing processes.

In other embodiments, the compositions are concentrated, e.g., a concentration in the range of about 10 to 500 mg/ml, about 10 to 400 mg/ml, about 10 to 300 mg/ml, about 10 to 200 mg/ml, about 10 to 180 mg/ml, about 40 to 150 mg/ml, about 60 to 125 mg/ml, or about 90 to 100 mg/ml, in aqueous solution (e.g., an isotonic or high salt aqueous solution) in a pH ranging from about 5 to 8).

In another aspect, the invention features methods and compositions for use in the prevention and/or treatment of viral infection comprising a modified anti-viral and/or anti-fusogenic peptide or conjugate thereof, as described herein. The method includes administering to a subject (e.g., a human subject) in need to treatment an effective amount, e.g., a prophylactic or therapeutic amount, of a modified anti-viral and/or anti-fusogenic peptide or conjugate thereof, as described herein to reduce one or more symptoms associated with the viral infection. Exemplary viral infections that can be treated or prevented include AIDS, human respiratory syncytial virus (RSV), human parainfluenza virus (HPV), measles virus (MeV) and simian immunodeficiency virus (SIV). Thus, methods for reducing or inhibiting, or preventing or delaying the onset of, one or more symptoms of a viral-associated disorder or condition using the modified anti-viral and/or anti-fusogenic peptides, or conjugates thereof, are disclosed. In the case of prophylactic use (e.g., to prevent, reduce or delay onset or recurrence of one or more symptoms of the disorder or condition), the subject may or may not have one or more symptoms of the disorder or condition. For example, the modified anti-viral and/or anti-fusogenic peptide or conjugate thereof can be administered prior to any detectable manifestation of the symptoms, or after at least some, but not all the symptoms are detected. In the case of therapeutic use, the treatment may improve, cure, maintain, or decrease duration of, the disorder or condition in the subject. In therapeutic uses, the subject may have a partial or full manifestation of the symptoms. In a typical case, treatment improves the disorder or condition of the subject to an extent detectable by a physician, or prevents worsening of the disorder or condition.

Methods and compositions for inhibiting one or more activities of HIV, RSV, HPV, MeV or SIV in a subject, e.g., a human subject, are disclosed. The method includes administering to a subject in need to treatment an effective amount, e.g., a prophylactic or therapeutic amount, of a modified anti-viral and/or anti-fusogenic peptide or a conjugate thereof, as described herein.

The modified peptides of the invention are also useful in facilitating purification and manufacturing process since the increased solubility of the modified peptides allows for more concentrated reacting solutions, thus facilitating large-scale manufacturing processes. Accordingly, the invention also features a method for enhancing the solubility of an antiviral and/or anti-fusogenic peptide. The method includes providing a modified antiviral and/or anti-fusogenic peptide containing one or more polar moieties (e.g., one or more cysteic acids), e.g., a modified peptide as described herein; and preparing a solution of the modified peptide (e.g., a pharmaceutical composition as described herein, or a manufacturing preparation). The method can, optionally, include determining the solubility of the modified antiviral and/or anti-fusogenic peptide in solution (e.g., by obtaining a sample of the modified antiviral and/or anti-fusogenic peptide in solution, and evaluating the turbidity and/or opalescence of the sample).

In another aspect, the invention features a method for enhancing the preparation, e.g., conjugaton (e.g., large-scale conjugation), of an antiviral and/or anti-fusogenic peptide. The method includes providing a modified antiviral and/or anti-fusogenic peptide containing one or more polar moieties (e.g., one or more cysteic acids), e.g., a modified peptide as described herein; and preparing a solution of the modified peptide that has a high concentration of the modified peptide (e.g., a high concentration as described herein).

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The contents of all publications, pending patent applications, published patent applications (inclusive of WO 02/096935 and US 05/0070475), and published patents cited throughout this application are hereby incorporated by reference in their entirety.

Others features, objects and advantages of the invention will be apparent from the description and drawings, and from the claims.

### BRIEF DESCRIPTION OF THE DRAWINGS

FIG. 1 is a linear graph depicting the inhibition of HIV-1_{IIIB} replication in peripheral blood mononuclear cells (PBMC) in the presence of control (filled diamonds) compared to native C34 (open squares).

FIG. 2 is a linear graph depicting the inhibition of HIV-1_{III}B replication in PBMC in the presence of control (filled diamonds) compared to C34-Lys³⁵ (ε-AEEA-MPA) conjugated to human serum albumin (C34-Lys³⁵ (F-AEEA-MPA):HSA)(open squares).

FIG. 3 is a linear graph depicting the inhibition of HIV-1_{III}B replication in PBMC in the presence of control (filled diamonds) compared to the albumin conjugate of C34 having a cysteic acid at the N-terminal end, and AEEA-MPA attached to the epsilon NH₂ of lysine added at the C-terminal end (CA-C34-Lys³⁵ (ε-AEEA-MPA) conjugated to human serum albumin (CA-C34-Lys³⁵ (ε-AEEA-MPA):HSA)(open squares).

FIG. 4 is a linear graph depicting the inhibition of HIV-1_{III}B replication in PBMC in the presence of control (filled diamonds) compared to conjugate of albumin coupled to the N-terminal α-amino group of tryptophan of C34 via a MPA-AEEA linker ((also referred to therein as PC-1505; MPA-(AEEA)-C34) (open squares).

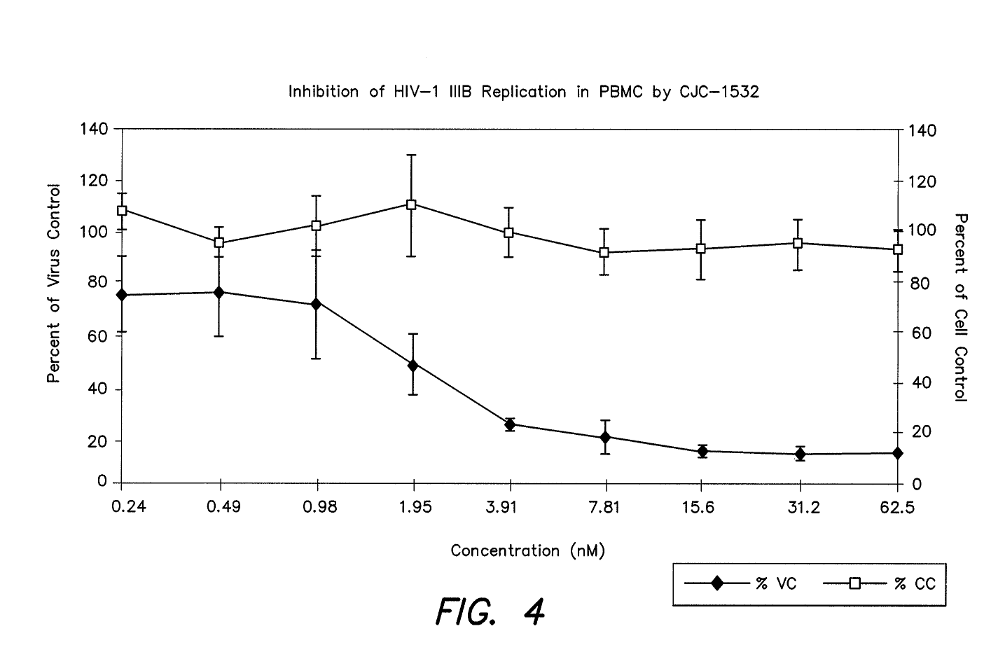

FIG. 5A illustrates pharmacokinetic curves of C34 peptide and Compound VIII (also referred to therein as PC-1505; MPA-(AEEA)-C34; and AC-CpdVIII) following either intravenous or subcutaneous administration into Sprague-Dawley rats.

FIG. 5B illustrates pharmacokinetic curve of Compound VIII as compared to that of rHA following either intravenous or subcutaneous administration into Sprague-Dawley rats. The superimposition of the curves provides definitive supporting evidence for the stability of the chemical bond linking maleimido-Compound VIII to cysteine-34 of human serum albumin as well as the stability of Compound VIII against renal clearance and peptidase degradation.

FIG. 6 is a table summarizing the results of the activity of several modified anti-fusogenic peptides in PBMC using HIV_{IIIb}.

### DETAILED DESCRIPTION OF THE INVENTION

Modified anti-viral and/or anti-fusogenic peptides having increased solubility in aqueous solution at physiological pH, compared to the peptides prior to modification, are disclosed. In one embodiment, the peptides of the invention are modified to include one or more polar moieties, e.g., one or more cysteic acids, thereby increasing their solubilities in aqueous solutions. The modified peptides can further include chemically reactive moieties such that the modified peptides can react with available functionalities on blood components or carrier proteins, e.g., albumin, thus increasing the stability in vivo of the modified peptides. The modified peptides of the present invention can be used, e.g., prophylactically against and/or therapeutically for ameliorating infection of a number of viruses, including human immunodeficiency virus (HIV), human respiratory syncytial virus (RSV), human parainfluenza virus (HPV), measles virus (MeV) and simian immunodeficiency virus (SIV).

Certain terms are defined herein as follows:

Anti-viral peptides: As used herein, “anti-viral peptides” shall refer to peptides that inhibit viral infection of cells, by, for example, inhibiting cell-cell fusion or free virus infection. The route of infection may involve membrane fusion, as occurs in the case of enveloped viruses, or some other fusion event involving viral and cellular structures. Peptides that inhibit viral infection by a particular virus may be referenced with respect to that particular virus, e.g., anti-HIV peptide, anti-RSV peptide, among others.

Antifusogenic peptides: “Anti-fusogenic peptides” are peptides demonstrating an ability to inhibit or reduce the level of membrane fusion events between two or more entities, e.g., virus-cell or cell-cell, relative to the level of membrane fusion that occurs in the absence of the peptide.

HIV and anti-HIV peptides: The human immunodeficiency virus (HIV), which is responsible for acquired immune deficiency syndrome (AIDS), is a member of the lentivirus family of retroviruses. There are two prevalent types of HIV, HIV-1 and HIV-2, with various strain of each having been identified. HIV targets CD-4+ cells, and viral entry depends on binding of the HIV protein gp41 to CD-4+ cell surface receptors. Anti-HIV peptides refer to peptides that exhibit anti-viral activity against HIV, including inhibiting CD-4+ cell infection by free virus and/or inhibiting HIV-induced syncytia formation between infected and uninfected CD-4+ cells.

SIV and anti-SIV peptides: Simian immunodeficiency viruses (SIV) are lentiviruses that cause acquired immunodeficiency syndrome (AIDS)-like illnesses in susceptible monkeys. Anti-SIV peptides are peptides that exhibit anti-viral activity against SIV, including inhibiting of infection of cells by the SIV virus and inhibiting syncytia formation between infected and uninfected cells.

RSV and anti-RSV peptides: Respiratory syncytial virus (RSV) is a respiratory pathogen, especially dangerous in infants and small children where it can cause bronchiolitis (inflammation of the small air passages) and pneumonia. RSVs are negative sense, single stranded RNA viruses and are members of the Paramyxoviridae family of viruses. The route of infection of RSV is typically through the mucous membranes by the respiratory tract, i.e., nose, throat, windpipe and bronchi and bronchioles. Anti-RSV peptides are peptides that exhibit anti-viral activity against RSV, including inhibiting mucous membrane cell infection by free RSV virus and syncytia formation between infection and uninfected cells.

HPV and anti-HPV peptides: Human parainfluenza virus (HPIV or HPV), like RSV, is another leading cause of respiratory tract disease, and like RSVs, are negative sense, single stranded RNA viruses that are members of the Paramyxoviridae family of viruses. There are four recognized serotypes of HPIV—HPIV-1, HPIV-2, HPIV-3 and HPIV-4. HPIV-1 is the leading cause of croup in children, and both HPIV-1 and HPIV-2 cause upper and lower respiratory tract illnesses. HPIV-3 is more often associated with bronchiolitis and pneumonia. Anti-HPV peptides are peptides that exhibit anti-viral activity against HPV, including inhibiting infection by free HPV virus and syncytia formation between infected and uninfected cells.

MeV and anti-Mev peptides: Measles virus (VM or MeV) is an enveloped negative, single-stranded RNA virus belonging to the Paramyxoviridae family of viruses. Like RSV and HPV, MeV causes respiratory disease, and also produces an immuno-suppression responsible for additional, opportunistic infections. In some cases, MeV can establish infection of the brain leading to severe neurlogical complications. Anti-MeV peptides are peptides that exhibit anti-viral activity against MeV, including inhibiting infection by free MeV virus and syncytia formation between infected and uninfected cells.

C34 and C34 analogs: The term “C34” refers to a portion of a gp41 coiled-coil cavity binding residues. For example, the peptide can include residues ⁶²⁸WMEW⁶³¹ of gp41 (SEQ ID NO:1), or ⁶²⁸WMEWDREINNYTSLIHSLIEESQNQQEKNEQELL⁶⁶¹ of gp41 (SEQ ID NO:2).

Analogs of C34 can include truncations, deletions, insertions and/or amino acid substitutions (e.g., conservative or non-conservative substitution) thereof. Deletions may consist of the removal of one or more amino acid residues from the C34 peptide, and may involve the removal of a single contiguous portion of the peptide sequence or multiple portions. Insertions may comprise single amino acid residues or stretches of residues and may be made at the carboxy or amino terminal end of the C34 peptide or at a position internal to the peptide.

DP-178 and DP178 analogs: Unless otherwise indicated explicitly or by context, DP-178 means the 36 amino acid DP-178 peptide corresponding to amino acid residues 638-673 of the gp41 glycoprotein of HIV-1 isolate LAI (HIV_{LAI}) and having the sequence:

(SEQ ID NO:7)

YTSLIHSLIEESQNQQEKNEQELLELDKWASLWNWF

Analogs of DP178 can include truncations, deletions, insertions and/or amino acid substitutions (e.g., conservative or non-conservative substitution) thereof. Truncations of the peptide may comprise peptides of between 3-36 amino acids. Deletions may consist of the removal of one or more amino acid residues from the DP178 peptide, and may involve the removal of a single contiguous portion of the peptide sequence or multiple portions. Insertions may comprise single amino acid residues or stretches of residues and may be made at the carboxy or amino terminal end of the DP178 peptide or at a position internal to the peptide.

DP178 peptide analogs are peptides whose amino acid sequences are comprised of the amino acid sequences of peptide regions of viruses other than HIV-1_{LAI} that correspond to the gp41 region from which DP178 was derived, as well as an truncations, deletions or insertions thereof. Such other viruses may include, but are not limited to, other HIV isolates such as HIV-2_{NIHZ}, respiratory syncytial virus (RSV), human parainfluenza virus (HPV), simian immunodeficiency virus (SIV), and measles virus (MeV). DP178 analogs also refer to those peptide sequences identified or recognized by the ALLMOTI5, 107×178×4 and PLZIP search motifs described in U.S. Pat. Nos. 6,013,263, 6,017,536 and 6,020,459 and incorporated herein, having structural and/or amino acid motif similarity to DP178. DP178 analogs further refer to peptides described as “DP178-like” as that term is defined in U.S. Pat. Nos. 6,013,263, 6,017,536 and 6,020,459.

DP-107 and DP 107 analogs: Unless otherwise indicated explicitly or by context, DP-107 means the 38 amino acid DP-107 peptide corresponding to amino acid residues 558-595 of the gp41 protein of HIV-1 isolate LAI (HIV_{LAI}) and having the sequence:

(SEQ ID NO:8)

NNLLRAIEAQQHLLQLTVWQIKQLQARILAVERYLKDQ.

Analogs of DP107 can include truncations, deletions, insertions and/or amino acid substitutions (e.g., conservative or non-conservative substitution) thereof. Truncations of the peptide may comprise peptides of between 3-38 amino acids. Deletions may consist of the removal of one or more amino acid residues from the DP107 peptide, and may involve the removal of a single contiguous portion of the peptide sequence or multiple portions. Insertions may comprise single amino acid residues or stretches of residues and may be made at the carboxy or amino terminal end of the DP107 peptide or at a position internal to the peptide.

DP107 peptide analogs are peptides whose amino acid sequences are comprised of the amino acid sequences of peptide regions of viruses other than HIV-1_{LAI} that correspond to the gp41 region from which DP107 was derived, as well as truncations, deletions and/or insertions thereof. Such other viruses may include, but are not limited to, other HIV isolates such as HIV-2_{NIHZ}, respiratory syncytial virus (RSV), human parainfluenza virus (HPV), simian immunodeficiency virus (SIV), and measles virus (MeV). DP107 analogs also refer to those peptide sequences identified or recognized by the ALLMOTI5, 107×178×4 and PLZIP search motifs described in U.S. Pat. Nos. 6,013,263, 6,017,536 and 6,020,459 and incorporated herein, having structural and/or amino acid motif similarity to DP107. DP107 analogs further refer to peptides described as “DP107-like” as that term is defined in U.S. Pat. Nos. 6,013,263, 6,017,536 and 6,020,459.

Reactive Groups: Reactive groups are chemical groups capable of forming a covalent bond. Such reactive groups are coupled or bonded to a C34, DP-107, DP-178 or T-1249 peptide or analogs thereof or other anti-viral or anti-fusogenic peptide of interest. Reactive groups will generally be stable in an aqueous environment and will usually be carboxy, phosphoryl, or convenient acyl group, either as an ester or a mixed anhydride, or an imidate, thereby capable of forming a covalent bond with functionalities such as an amino group, a hydroxy or a thiol at the target site on mobile blood components. For the most part, the esters will involve phenolic compounds, or be thiol esters, alkyl esters, phosphate esters, or the like.

Functionalities: Functionalities are groups on blood components to which reactive groups on modified anti-viral peptides react to form covalent bonds. Functionalities include hydroxyl groups for bonding to ester reactive entities; thiol groups for bonding to maleimides, imidates and thioester groups; amino groups for bonding to carboxy, phosphoryl or acyl groups and carboxyl groups for bonding to amino groups.

Blood Components or Carrier Proteins: Blood components may be either fixed or mobile. Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems. Mobile blood components are blood components that do not have a fixed situs for any extended period of time, generally not exceeding 5, more usually one minute. These blood components are not membrane-associated and are present in the blood for extended periods of time and are present in a minimum concentration of at least 0.1.mu.g/ml. Mobile blood components include carrier proteins. Mobile blood components include serum albumin, transferrin, ferritin and immunoglobulins such as IgM and IgG. The half-life of mobile blood components is at least about 12 hours. Additional examples of blood components include ferritin, steroid binding proteins, transferrin, thyroxin binding protein, and α-2-macroglobulin. Typically, serum albumin and IgG being more preferred, and serum albumin, e.g., human serum albumin being the most preferred. Albumin may also be derived from a recombinant or genomic source, such as yeast, bacteria (e.g., E. coli), mammalian cells (e.g., Chinese hamster ovary (CHO) cells), transgenic plant, transgenic animal, Thus, the term “blood component” includes proteins that are biochemically purified from a subject, as well as proteins made recombinantly.

Protective Groups: Protective groups are chemical moieties utilized to protect peptide derivatives from reacting with themselves. Various protective groups are disclosed herein and in U.S. Pat. No. 5,493,007, which is hereby incorporated by reference. Such protective groups include acetyl, fluorenylmethyloxycarbonyl (Fmoc), t-butyloxycarbonyl (B oc), benzyloxycarbonyl (CBZ), and the like. The specific protected amino acids are depicted in Table 1.

TABLE 1
NATURAL AMINO ACIDS AND THEIR ABBREVIATIONS

| | 3-Letter | 1-Letter | |
|---|---|---|---|
| Name | Abbreviation | Abbreviation | Modified Amino Acids |
| Alanine | Ala | A | Fmoc-Ala-OH |
| Arginine | Arg | R | Fmoc-Arg(Pbf)-OH |
| Asparagine | Asn | N | Fmoc-Asn(Trt)-OH |
| Aspartic acid | Asp | D | Asp(tBu)-OH |
| Cysteine | Cys | C | Fmoc-Cys(Trt) |
| Glutamic acid | Glu | E | Fmoc-Glu(tBu)-OH |
| Glutamine | Gln | Q | Fmoc-Gln(Trt)-OH |
| Glycine | Gly | G | Fmoc-Gly-OH |
| Histidine | His | H | Fmoc-His(Trt)-OH |
| Isoleucine | Ile | I | Fmoc-Ile-OH |
| Leucine | Leu | L | Fmoc-Leu-OH |
| Lysine | Lys | Z | Boc-Lys(Aloc)-OH |
| Lysine | Lys | X | Fmoc-Lys(Aloc)-OH |
| Lysine | Lys | K | Fmoc-Lys(Mtt)-OH |
| Methionine | Met | M | Fmoc-Met-OH |
| Phenylalanine | Phe | F | Fmoc-Phe-OH |
| Proline | Pro | P | Fmoc-Pro-OH |
| Serine | Ser | S | Fmoc-Ser(tBu)-OH |
| Threonine | Thr | T | Fmoc-Thr(tBu)-OH |
| Tryptophan | Trp | W | Fmoc-Trp(Boc)-OH |
| Tyrosine | Tyr | Y | Boc-Tyr(tBu)-OH |
| Valine | Val | V | Fmoc-Val-OH |

Linking Groups Linking (spacer) groups are chemical moieties that link or connect reactive entities to antiviral or antifusogenic peptides. Linking groups may comprise one or more alkyl moeities, alkoxy moeity, alkenyl moeity, alkynyl moeity or amino moeity substituted by alkyl moeities, cycloalkyl moeity, polycyclic moeity, aryl moeity, polyaryl moeities, substituted aryl moeities, heterocyclic moeities, and substituted heterocyclic moeities. Linking groups may comprise (2-amino)ethoxy acetic acid (AEA), [2-(2-amino)ethoxy)]ethoxy acetic acid (AEEA), ethylenediamine (EDA); one or more alkyl chains (C₁-C₁₀) such as 8-aminooctanoic acid (AOA), 8-aminopropanoic acid (APA), or 4-aminobenzoic acid (APhA).

Sensitive Functional Groups: A sensitive functional group is a group of atoms that represents a potential reaction site on an antiviral and/or antifusogenic peptide. If present, a sensitive functional group may be chosen as the attachment point for the linker-reactive group modification. Sensitive functional groups include but are not limited to carboxyl, amino, thiol, and hydroxyl groups.

Modified Peptides: A modified peptide is an antiviral and/or antifusogenic peptide that has been modified by attaching a reactive group. The reactive group may be attached to the peptide either via a linking group, or optionally without using a linking group. It is also contemplated that one or more additional amino acids may be added to the peptide to facilitate the attachment of the reactive entity. Modified peptides may be administered in vivo such that conjugation with blood components occurs in vivo, or they may be first conjugated to blood components or carrier proteins in vitro (e.g., using recombinantly produced proteins, such as recombinant albumin, immunoglobulin, or transferring) and the resulting conjugated peptide (as defined below) administered in vivo.

Conjugated Peptides: A conjugated peptide is a modified peptide that has been conjugated to a blood component via a covalent bond formed between the reactive group of the modified peptide and the functionalities of the blood component, with or without a linking group. As used throughout this application, the term “conjugated peptide” can be made more specific to refer to particular conjugated peptides, for example “conjugated C34” or “conjugated DP107.”

In embodiments, the modified anti-viral and/or anti-fusogenic peptides of the invention include a maleimide containg group which has the ability to covalently bond blood components and more particularly serum albumin so as to form a conjugate. The administration of a maleimide derivative of an anti-viral and/or anti-fusogenic peptide to a subject can result in the in vivo conjugation of the peptide to a blood component such as serum albumin. It is also encompassed by the present invention to prepare the conjugate ex vivo (or in vivo) by contacting the modified anti-viral and/or anti-fusogenic peptidewith a blood component or carrier protein, e.g., albumin. In this case, albumin can be provided from different sources, e.g., in blood samples, purified albumin, recombinant albumin (including modified forms of albumin, e.g., having amino acid substitutions, insertions and/or deletions) or the like. The preparation and use of conjugates of C34 and albumin have been thoroughly disclosed in WO 02/096935, and similar preparations and uses apply to conjugates of the present invention. The conjugates formed in vivo in a subject and the ex vivo prepared conjugates when administered to a subject are both useful for exhibiting anti-fusogenic activity of the corresponding fusion peptide inhibitor an, therefore, inhibiting the activity of HIV, RSV, HPV, MeV or SIV in a subject.

Taking into account these definitions, the present invention takes advantage of the properties of existing anti-viral and antifusogenic peptides. The viruses that may be inhibited by the peptides include, but are not limited to all strains of viruses listed, e.g., in U.S. Pat. Nos. 6,013,263, 6,017,536 and 6,020,459 at Tables V-VII and IX-XIV therein. These viruses include, e.g., human retroviruses, including HIV-1, HIV-2, and human T-lympocyte viruses (HTLV-I and HTLV-II), and non-human retroviruses, including bovine leukosis virus, feline sarcoma virus, feline leukemia virus, simian immunodeficiency virus (SIV), simian sarcoma virus, simian leukemia, and sheep progress pneumonia virus. Non-retroviral viruses may also be inhibited by the peptides of the present invention, including human respiratory syncytial virus (RSV), canine distemper virus, Newcastle Disease virus, human parainfluenza virus (HPIV), influenza viruses, measles viruses (MeV), Epstein-Barr viruses, hepatitis B viruses, and simian Mason-Pfizer viruses. Non-enveloped viruses may also be inhibited by the peptides of the present invention, and include, but are not limited to, picornaviruses such as polio viruses, hepatitis A virus, enteroviruses, echoviruses, coxsackie viruses, papovaviruses such as papilloma virus, parvoviruses, adenoviruses, and reoviruses.

As an example, the mechanism of action of HIV fusion peptides has been described as discussed in the background section of this application and antiviral and antifusogenic properties of the peptides have been well established. A synthetic peptide corresponding to the carboxyl-terminal ectodomain sequence (for instance, amino acid residues 643-678 of HIV-1 class B, of the LAI strain or residues 638-673 from similar strain as well as residues 558-595) has been shown to inhibit virus-mediated cell-cell fusion completely at low concentration. The peptides of the invention compete with the leucine zipper region of the native viral gp41 thus resulting in the interference of the fusion/infection of the virus into the cell.

The invention additionally provides methods and reagents used to modify a selected anti-viral and/or antifusogenic peptide with the DAC™ (Drug Activity Complex) technology to confer to this peptide improved bio-availability, extended half-life and better distribution through selective conjugation of the peptide onto a protein carrier but without modifying the peptide's anti-viral properties. The carrier of choice (but not limited to) for this invention would be albumin conjugated through its free thiol by an anti-viral and/or antifusogenic peptide modified with a maleimide moiety.

Anti-Viral and/or Anti-Fusogenic Inhibitors

Several peptide sequences have been described in the literature as highly potent for the prevention of HIV-1 fusion/infection. As examples, peptides C34, DP107, DP178 binds to a conformation of gp41 that is relevant for fusion. Thus, in one embodiment of the invention, C34-, DP178- and DP178-like peptides are modified. Likewise, other embodiments of the invention include modification of C34-, DP107 and DP107-like peptide for use against HIV, as well as peptides analagous to DP107 and DP178 that are found in RSV, HPV, MeV and SIV viruses.

### Modified C34 Peptides or Analogues

In certain embodiments, the modified C34 peptides of the invention include additional group for inclusion in the peptide may be a compound having formula (I).

(R₁)ₘ-X-(R₂)ₙ (VIII)

In formula (VIII), the sum of m and n is at least 1 and m and n are each integers that are zero or greater. For example, where m is zero, then n is 1 or greater, and where n is zero, then m is 1 or greater. X is a peptide, peptide fragment or protein such as, for example, C34, T20, T1249 or derivatives thereof including, for example, maleimide derivatives thereof. Where R₁ is present and R₂ is absent, R₁ is present at the N-terminus of the X group. When R₁ is absent and R₂ is present, R₂ is present at the C-terminus of the X group.

In certain examples, R₁ and R₂ may each be independently selected from a compound having formula (IX).

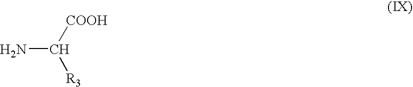

The core structure of formula (IX) is similar to that of an amino acid and includes an amino group, an alpha carbon and a carboxyl group. Depending on the exact position of the R₁ and R₂ groups in the peptide derivative, the groups may be bound to the peptide through different atoms of formula (IX). For example, where R₁ is a compound having formula (IX), R₁ may be bound to the peptide through the carboxyl group of formula (IX) to provide a peptide bond between the carboxyl group of R₁ and an amino group of the peptide. Where R₂ is a compound having formula (IX), R₂ may be bound to the peptide through the amino group of formula (IX) to provide a peptide bond between the amino group of R₂ and a carboxy group of the peptide.

In some examples, the R₃ group of formula (IX) may be any polar, uncharged group other than the polar, uncharged groups commonly found in the 20 naturally occurring amino acids. For example, the R₃ group may be, or may include, a sulfonyl group (HS═(O)₂), a sulfoxide group (HS═O), a sulfonic acid group (HO—S═(O)₂), a haloalkyl group, a secondary amine, a tertiary amine, a hydroxyl group, or other side chain group that is polar or even neutral and that can increase the overall solubility of the peptide derivative in an aqueous solution. For example, a side chain with groups capable of hydrogen bonding may be used to increase the overall solubility of the peptide. In certain examples, the side chain is preferably non-reactive such that unwanted side reactions with a linker or other species do not occur to any substantial degree. In some examples, the above-noted groups for R₃ may be spaced from the alpha carbon, for example, by 1-3 carbon atoms.

In certain examples, R₃ may be selected to provide a compound having formulae (X)-(XV).

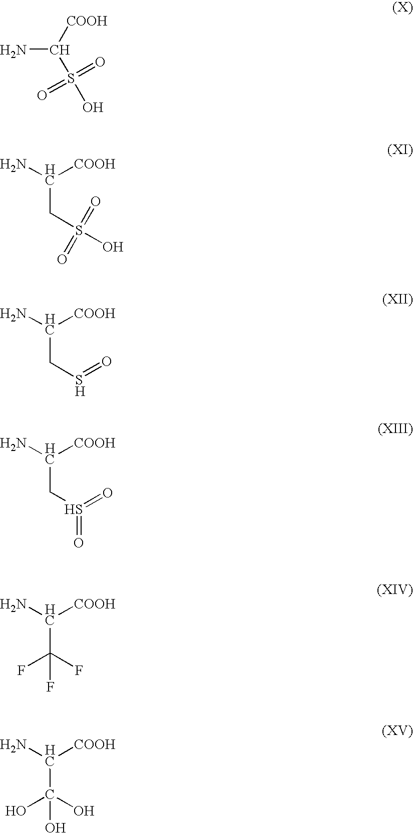

Additional suitable side chains that can increase the solubility of the peptides disclosed herein will be readily selected by the person of ordinary skill in the art, given the benefit of this disclosure.

In certain embodiments, the R₁ and R₂ groups do not substantially affect the overall secondary, or in certain instances the tertiary structure, of the peptide conjugate. By not substantially affecting the secondary structure of the peptide conjugate, the overall activity of the peptide conjugate should not be appreciably less than that of the non-derivatized peptide.

In other embodiments, the peptide derivative may take the form of a composition as shown in formula (XVI).

X₁-(R₁)ₘ-X₂-(R2)ₙ (XVI)

In formula (XVI), X₁ and X₂ represent portions of a peptide that when joined together would provide, for example, C34, T20, or T1249. In formula (XVI), R₁ and R₂ may be any of those groups discussed above in reference to formula (IX), and the sum of m and n is an integer greater than or equal to 1, with the possibility that either m or n may be zero. In formula (XVI), the group has been inserted into the middle of the peptide chain. Such insertion may be performed using many different methods including enzymatic digestion of the peptide, followed by insertion of an R₁ or R₂ group or both and then subsequent attachment of the peptide fragments together.

Synthesis of cysteic acid derivatives of C34 described herein is performed using an automated solid-phase procedure on a Symphony Peptide Synthesizer with manual intervention during the generation of the peptide (see Examples 1-5 herein). The synthesis was performed on Fmoc-protected Ramage amide linker resin, using Fmoc-protected amino acids. Coupling was achieved by using O-benzotriazol-1-yl-N,N,N′,N′-tetramethyl-uronium hexafluorophosphate (HBTU) and diisopropylethylamine (DIEA) as the activator cocktail in N,N-dimethylfonmamide (DMF) solution. The Fmoc protective group was removed using 20% piperidine/DMF. A Boc-protected amino acid was used at the N-terminus in order to generated the free N_{α}-terminus once the peptides were cleaved from the resin. Sigmacoted glass reaction vessels were used during the synthesis.

In certain embodiments, a portion of the peptide may be synthesized using conventional solid phase synthesis techniques as described for example by Merrifield, 1986. Solid phase synthesis. Science. 232: 341-347. In brief, a blocking group is added to the N-terminus of an amino acid, and the carboxyl group of the amino acid may be activated by reaction with dicyclohexylcarbodiimide (DCCD). The activated amino acid may be reacted with an amino acid having a free N-terminus and a C-terminus bound to a resin or bead. After formation of an amino-blocked dipeptidyl compound, acid treatment results in production of isobutylene, carbon dioxide and a dipeptide bound to the resin or bead. Additional amino acids may be added to the dipeptide bead by repeating these steps. In addition, the amino acid derivatives disclosed herein may also be added to the peptide chain, at any point along the chain, using similar reactions. Thus, it is possible to insert R₁ or R₂ groups anywhere at a position in a desired peptide to provide a compound having formula (IX).

In certain embodiments, the compounds disclosed herein may be linked to one or more additional groups at the N-terminus, the C-terminus or through a side chain of one or more of the amino acids of the peptide. For example, compositions as shown schematically in formulae (XVII)-(XX) may be produced.

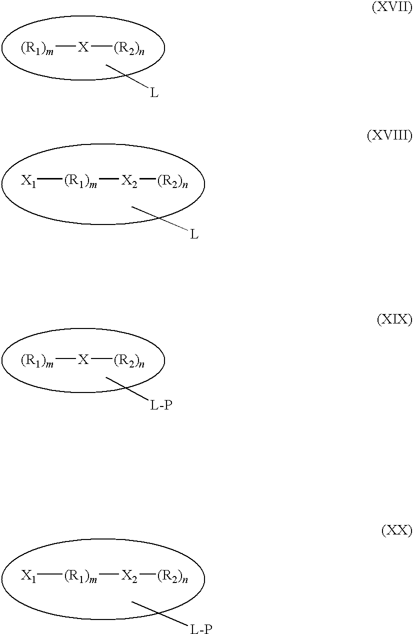

In formulas (XVII)-(XX), L is a linker such as, for example, (2-amino)ethoxy acetic acid (AEA), ethylenediamine (EDA), 2-[2-(2-amino)ethoxy]ethoxy acetic acid (AEAA), alkyl chain motifs (C1-C10) such as glycine, 3-aminopropionic acid (APA), 8-aminooctoanic acid (AOA), 4-aminobenzoic acid (APhA) or the like, and R₁ and R₂ may be any of those groups discussed herein. The linker may be bound to the peptide through any amino acid of the peptide, for example, through an epsilon amino group of a lysine in the peptide, at the N-terminus or at the C-terminus of the peptide. The X, X₁ and X₂ groups are a peptide (X) or peptide fragments (X₁ and X₂). The P group shown in formulae (XIX) and (XX) represents a protein that may be conjugated to the derivatized peptide through the linker L. Illustrative proteins include, a blood protein, human serum albumin, recombinant albumin or other suitable proteins.

The protein conjugates (formulae (XIX) and (XX)) may be produced ex vivo or in vivo. Where in vivo production occurs, compounds, such as those shown in formulae (XVII) and (XVIII), may be introduced into a subject and react with an in vivo protein such as albumin.

Non-limiting examples of modified anti-viral and/or anti-fusogenic modified peptides of C34 of the present invention include the following sequences:

CA Compound I: (Cysteic Acid (CA) directly linked to C34; also referred to herein as CA-C34 (SEQ ID NO:3).

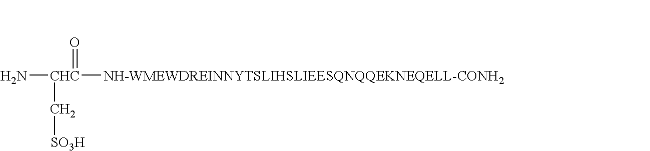

CA Compound II: (Cysteic Acid (CA) directly linked to C34 having a substitution of native Lysine at position 28 (Lys²⁸) for an arginine; also referred to herein as CA-C34 (Arg²⁸) (SEQ ID NO:4).

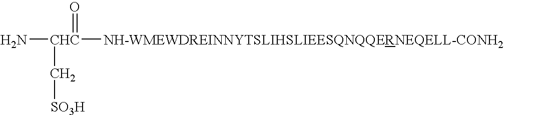

CA Compound III: (Cysteic Acid (CA) directly linked to C34 having an additional Lysine residue at position 35 (Lys³⁵), wherein the epsilon NH₂ group of lysine is coupled to the reactive group via linker (AEEA-MPA); also referred to herein as CA-C34-Lys³⁵ (ε-AEEA-MPA) (SEQ ID NO:5).

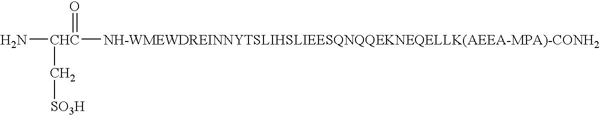

CA Compound IV: (Cysteic Acid (CA) directly linked to C34 having a substitution of native Lysine at position 28 (Lys²⁸) for an arginine; an additional Lysine residue at position 35 (Lys³⁵), wherein the epsilon NH₂ group of lysine is coupled to the reactive group via linker (AEEA-MPA); also referred to herein as CA-C34 (Arg²⁸)-Lys³⁵ (ε-AEEA-MPA) (SEQ ID NO:6).

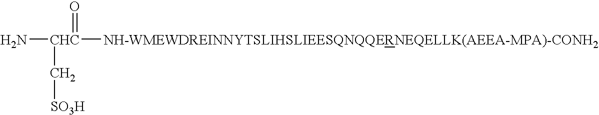

Additional examples of modified C34 peptides that can be modified following the teachings of the application also include the following amino acid sequences:

(SEQ ID NO:8)

N term-W M E W D R E I N N Y T S L I H S L I E E S
Q N Q Q E K N E Q E L L-C term;

(SEQ ID NO:9)

N term-W M E W D R E I N N Y T S L I H S L I E E S
Q N Q Q E R N E Q E L K-C term;

(SEQ ID NO:10)

N term-W M E W D R E I N N Y T S L I H S L I E E S
Q N Q Q E R N E Q E K L-C term;

(SEQ ID NO:11)

N term-W M E W D R E I N N Y T S L I H S L I E E S
Q N Q Q E R N E Q K L L-C term;

(SEQ ID NO:12)

N term-W M E W D R E I N N Y T S L I H S L I E E S
Q N Q Q E R N E K E L L-C term;

(SEQ ID NO:13)

N term-W M E W D R E I N N Y T S L I H S L I E E S
Q N Q Q E R N K Q E L L-C term;

(SEQ ID NO:14)

N term-W M E W D R E I N N Y T S L I H S L I E E S
Q N Q Q E R K E Q E L L-C term;

(SEQ ID NO:15)

N term-W M E W D R E I N N Y T S L I H S L I E E S
Q N Q Q E R N E Q E L L K-C term;
and

(SEQ ID NO:16)

N term-W M E W D R E I N N Y T S L I H S L I E E S
Q N Q Q E K N E Q E L L K-C term..

Non-limiting examples of modified C34 peptides are the compounds of Formulae I-VIII illustrated below, which are capable of reacting with thiol groups on a blood component either in vivo or ex vivo, to form a stable covalent bond. Synthesis of these compounds is described in WO 02/096935, the contents of which are hereby specifically incorporated by reference.

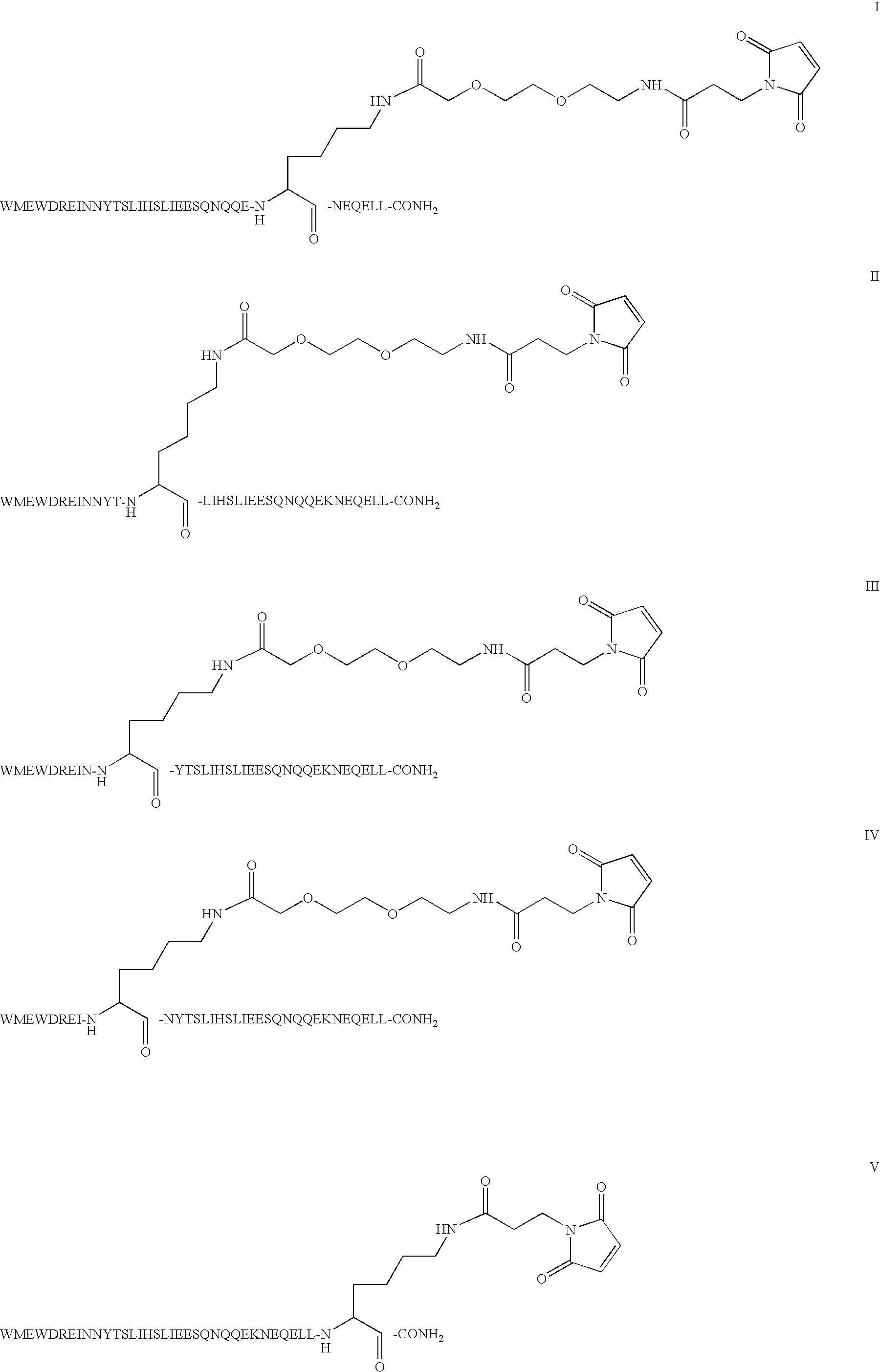

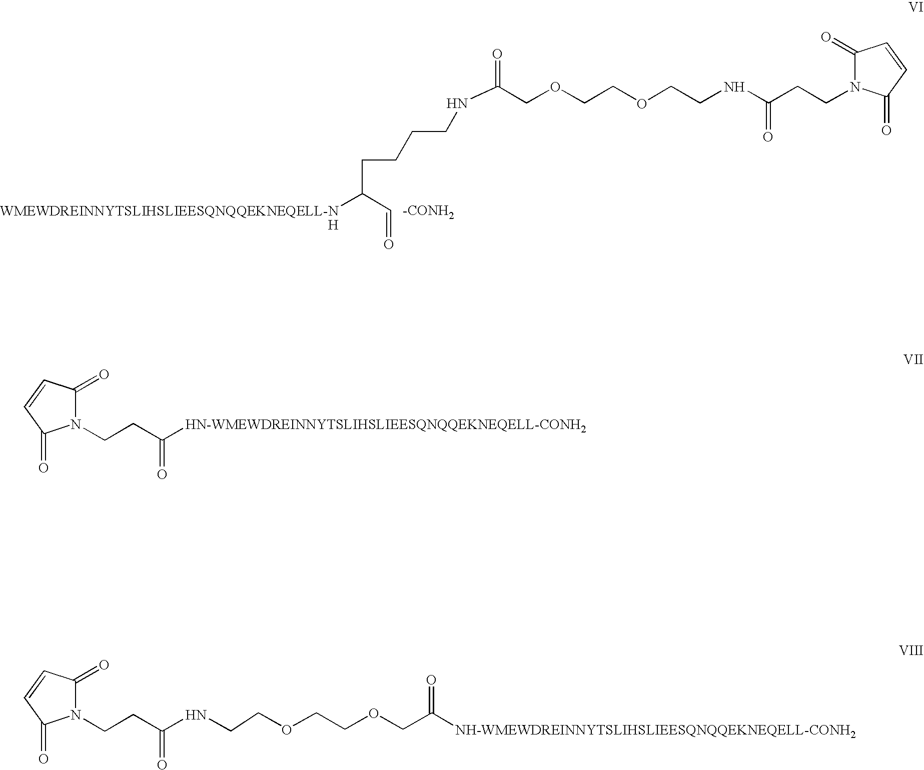

### DP178 and DP107

### DP178 Peptides

The DP178 peptide corresponds to amino acid residues 638 to 673 of the transmembrane protein gp41 from the HIV-1_{LAI} isolate, and has the 36 amino acid sequence (reading from amino to carboxy terminus):

(SEQ ID NO:7)

NH₂-YTSLIHSLIEESQNQQEKNEQELLELDKWASLWNWF-COOH

In addition to the full-length DP178 36-mer, the peptides of this invention include truncations of the DP178 peptide comprising peptides of between 3 and 36 amino acid residues (i.e., peptides ranging in size from a tripeptide to a 36-mer polypeptide), These truncated peptides are shown in Tables 2 and 3.

In addition amino acid substitutions of the DP178 peptide are also within the scope of the invention. HIV-1 and HIV-2 enveloped proteins are structurally distinct, but there exists a striking amino acid conservation within the DP178-corresponding regions of HIV-1 and HIV-2. The amino acid conservation is of a periodic nature, suggesting some conservation of structure and/or function. Therefore, one possible class of amino acid substitutions would include those amino acid changes which are predicted to stabilize the structure of the DP178 peptides of the invention. Utilizing the DP178 and DP178 analog sequences described herein, the skilled artisan can readily compile DP178 consensus sequences and ascertain from these, conserved amino acid residues which would represent preferred amino acid substitutions.

The amino acid substitutions may be of a conserved or non-conserved nature. Conserved amino acid substitutions consist of replacing one or more amino acids of the DP178 peptide sequence with amino acids of similar charge, size, and/or hydrophobicity characteristics, such as, for example, a glutamic acid (E) to aspartic acid (D) amino acid substitution. Non-conserved substitutions consist of replacing one or more amino acids of the DP178 peptide sequence with amino acids possessing dissimilar charge, size, and/or hydrophobicity characteristics, such as, for example, a glutamic acid (E) to valine (V) substitution.

Amino acid insertions of DP178 may consist of single amino acid residues or stretches of residues. The insertions may be made at the carboxy or amino terminal end of the DP178 or DP178 truncated peptides, as well as at a position internal to the peptide.

Such insertions will generally range from 2 to 15 amino acids in length. It is contemplated that insertions made at either the carboxy or amino terminus of the peptide of interest may be of a broader size range, with about 2 to about 50 amino acids being preferred. One or more such insertions may be introduced into DP178 or DP178 truncations, as long as such insertions result in peptides which may still be recognized by the 107×178×4, ALLMOTI5 or PLZIP search motifs described above.

Preferred amino or carboxy terminal insertions are peptides ranging from about 2 to about 50 amino acid residues in length, corresponding to gp41 protein regions either amino to or carboxy to the actual DP178 gp41 amino acid sequence, respectively. Thus, a preferred amino terminal or carboxy terminal amino acid insertion would contain gp41 amino acid sequences found immediately amino to or carboxy to the DP178 region of the gp41 protein.

Deletions of DP178 or DP178 truncations are also within the scope of this invention. Such deletions consist of the removal of one or more amino acids from the DP178 or DP178-like peptide sequence, with the lower limit length of the resulting peptide sequence being 4 to 6 amino acids.

Such deletions may involve a single contiguous or greater than one discrete portion of the peptide sequences. One or more such deletions may be introduced into DP178 or DP178 truncations, as long as such deletions result in peptides which may still be recognized by the 107×178×4, ALLMOTI5 or PLZIP search motifs described above.

### DP107 Peptides

DP107 is a 38 amino acid-peptide which exhibits potent antiviral activity, and corresponds to residues 558 to 595 of HIV-1_{LAI} isolate transmembrane (TM) gp41 glycoprotein, as shown here:

(SEQ ID NO:17)

NH₂-NNLLRAIEAQQHLLQLTVWQIKQLQARILAVERYLKDQ-COOH.

In addition to the full-length DP107 38-mer, the DP107 peptides include truncations of the DP107 peptide comprising peptides of between 3 and 38 amino acid residues (i.e., peptides ranging in size from a tripeptide to a 38-mer polypeptide). These peptides are shown in Tables 4 and 5 of US 2005/0070475.

In addition, amino acid substitutions of the DP178 peptide are also within the scope of the invention. As for DP178, there also exists a strong amino acid conservation within the DP107-corresponding regions of HIV-1 and HIV-2, again of a periodic nature, suggesting conservation of structure and/or function. Therefore, one possible class of amino acid substitutions includes those amino acid changes predicted to stabilize the structure of the DP107 peptides of the invention. Utilizing the DP107 and DP107 analog sequences described herein, the skilled artisan can readily compile DP107 consensus sequences and ascertain from these, conserved amino acid residues which would represent preferred amino acid substitutions.

The amino acid substitutions may be of a conserved or non-conserved nature. Conserved amino acid substitutions consist of replacing one or more amino acids of the DP107 peptide sequence with amino acids of similar charge, size, and/or hydrophobicity characteristics, such as, for example, a glutamic acid (E) to aspartic acid (D) amino acid substitution. Non-conserved substitutions consist of replacing one or more amino acids of the DP107 peptide sequence with amino acids possessing dissimilar charge, size, and/or hydrophobicity characteristics, such as, for example, a glutamic acid (E) to valine (V) substitution.

Amino acid insertions may consist of single amino acid residues or stretches of residues. The insertions may be made at the carboxy or amino terminal end of the DP107 or DP107 truncated peptides, as well as at a position internal to the peptide.

Such insertions will generally range from 2 to 15 amino acids in length. It is contemplated that insertions made at either the carboxy or amino terminus of the peptide of interest may be of a broader size range, with about 2 to about 50 amino acids being preferred. One or more such insertions may be introduced into DP107 or DP107 truncations, as long as such insertions result in peptides which may still be recognized by the 107×178×4, ALLMOTI5 or PLZIP search motifs described above.

Preferred amino or carboxy terminal insertions are peptides ranging from about 2 to about 50 amino acid residues in length, corresponding to gp41 protein regions either amino to or carboxy to the actual DP107 gp41 amino acid sequence, respectively. Thus, a preferred amino terminal or carboxy terminal amino acid insertion would contain gp41 amino acid sequences found immediately amino to or carboxy to the DP107 region of the gp41 protein.

Deletions of DP107 or DP107 truncations are also within the scope of this invention. Such deletions consist of the removal of one or more amino acids from the DP107 or DP107-like peptide sequence, with the lower limit length of the resulting peptide sequence being 4 to 6 amino acids.

Such deletions may involve a single contiguous or greater than one discrete portion of the peptide sequences. One or more such deletions may be introduced into DP107 or DP107 truncations, as long as such deletions result in peptides which may still be recognized by the 107×178×4, ALLMOTI5 or PLZIP search motifs.

DP107 and DP107 truncations are more fully described in U.S. Pat. No. 5,656,480.

### DP107 and DP178 Analogs

Peptides corresponding to analogs of the DP178, DP178 truncations, DP107 and DP107 truncation sequences of the invention, described, above, may be found in other viruses, including, for example, non-HIV-1 enveloped viruses, non-enveloped viruses and other non-viral organisms.

Such DP178 and DP107 analogs may, for example, correspond to peptide sequences present in transmembrane (“TM”) proteins of enveloped viruses and may, correspond to peptide sequences present in non enveloped and nonviral organisms. Such peptides may exhibit antifusogenic activity, antiviral activity, most particularly antiviral activity which is specific to the virus in which their native sequences are found, or may exhibit an ability to modulate intracellular processes involving coiled-coil peptide structures.

### DP178 Analogs

DP178 analogs are peptides whose amino acid sequences are comprised of the amino acid sequences of peptide regions of, for example, other (i.e., other than HIV-1) viruses that correspond to the gp41 peptide region from which DP178 was derived. Such viruses may include, but are not limited to, other HIV-1 isolates and HIV-2 isolates.

DP178 analogs derived from the corresponding gp41 peptide region of other (i.e., non HIV-1LAI) HIV-1 isolates may include, for example, peptide sequences as shown below.

(SEQ ID NO:18)

NH2-YTNTIYTLLEESQNQQEKNEQELLELDKWASLWNWF-COOH

(SEQ ID NO:19)

NH2-YTGIIYNLLEESQNQQEKNEQELLELDKWANLWNWF-COOH

(SEQ ID NO:10)

NH2-YTSLIYSLLEKSQIQQEKNEQELLELDKWASLWNWF-COOH

The peptides of SEQ ID NO:18, SEQ ID NO:19 and SEQ ID NO:20 are derived from HIV-1_{SF2}, HIV-1_{RF}, and HIV-1_{MN}, respectively. Other DP178 analogs include those derived from HIV-2, including the peptides of SEQ ID NO:6 and SEQ ID NO:7 of US 2005/0070475, which are derived from HIV-2_{ROD} and HIV-2_{NIHZ}, respectively. Still other useful analogs include the peptides of SEQ ID NO:8 and SEQ ID NO:9 of US 2005/0070475, which have been demonstrated to exhibit anti-viral activity.

In the present invention, it is preferred that the DP178 analogs represent peptides whose amino acid sequences correspond to the DP178 region of the gp41 protein, it is also contemplated that the peptides disclosed herein may, additionally, include amino sequences, ranging from about 2 to about 50 amino acid residues in length, corresponding to gp41 protein regions either amino to or carboxy to the actual DP178 amino acid sequence.

Table 6 and Table 7 of US 2005/0070475 show some possible truncations of the HIV-2_{NIHZ} DP178 analog, which may comprise peptides of between 3 and 36 amino acid residues (i.e., peptides ranging in size from a tripeptide to a 36-mer polypeptide). Peptide sequences in these tables are listed from amino (left) to carboxy (right) terminus.

### Additional DP178 Analogs and DP107 Analogs

DP178 and DPI 07 analogs are recognized or identified, for example, by utilizing one or more of the 107×178×4, ALLMOTI5 or PLZIP computer-assisted search strategies described above. The search strategy identifies additional peptide regions which are predicted to have structural and/or amino acid sequence features similar to those of DP107 and/or DP178.

The search strategies are described fully in the example presented in Section 9 of U.S. Pat. Nos. 6,013,263, 6,017,536 and 6,020,459. While this search strategy is based, in part, on a primary amino acid motif deduced from DPI 07 and DP178, it is not based solely on searching for primary amino acid sequence homologies, as such protein sequence homologies exist within, but not between major groups of viruses. For example, primary amino acid sequence homology is high within the TM protein of different strains of HIV-1 or within the TM protein of different isolates of simian immunodeficiency virus (SIV).

The computer search strategy disclosed in U.S. Pat. Nos. 6,013,263, 6,017,536 and 6,020,459 successfully identified regions of proteins similar to DP107 or DP178. This search strategy was designed to be used with a commercially-available sequence database package, preferably PC/Gene.

In U.S. Pat. Nos. 6,013,263, 6,017,536 and 6,020,459, a series of search motifs, the 107×178×4, ALLMOTI5 and PLZIP motifs, were designed and engineered to range in stringency from strict to broad, with 107×178×4 being preferred. The sequences identified via such search motifs, such as those listed in Tables V-XIV, of U.S. Pat. Nos. 6,013,263, 6,017,536 and 6,020,459 potentially exhibit antifusogenic, such as antiviral, activity, may additionally be useful in the identification of antifusogenic, such as antiviral, compounds.

### Other Anti-Viral Peptides

### Anti-RSV Peptides

Anti-RSV peptides include DP178 and/or DP107 analogs identified from corresponding peptide sequences in RSV which have further been identified to inhibit viral infection by RSV. Such peptides of interest include the peptides of Table 16 and peptides of SEQ ID NO: 10 to SEQ ID NO:30 of US 2005/0070475. Detailed protocols for synthesizing these peptides are disclosed in US 2005/0070475, the contents of which are hereby specifically incorporated by reference. Of particular interest are the following peptides:

| | |
|---|---|
| YTSVITIELSNIKLNKCNGAKVKLIKQELDKYK | (SEQ ID NO:21) |
| TSVITIELSNIKENKCNGAKVKLIKQELDKYKN | (SEQ ID NO:22) |
| VITIELSNIKENKCNGAKVKLIKQELDKYKNAV | (SEQ ID NO:23) |
| IALLSTNKAVVSLSNGVSVLTSKVLDLKNYIDK | (SEQ ID NO:24) |

The peptide of SEQ ID NO: 10 of US 2005/0070475 is derived from the F2 region of RSV and was identified in U.S. Pat. Nos. 6,103,236 and 6,020,459 using the search motifs described as corresponding to DP107 and DP178 peptides (i.e., “DP107/178 like”). The peptides of SEQ ID NO:21 to SEQ ID NO:23 each have amino acid sequences contained within the peptide of SEQ ID NO:10 and each has been shown to exhibit anti-RSV activity, in particular, inhibiting fusion and syncytia formation between RSV-infected and uninfected Hep-2 cells at concentrations of less than 50 μg/ml.

The peptide of SEQ ID NO: 11 of US 2005/0070475 is derived from the F1 region of RSV and was identified in U.S. Pat. Nos. 6,103,236 and 6,020,459 using the search motifs described as corresponding to DP107 (i.e., “DP107-like”). The peptide of SEQ ID NO:24 contains amino acid sequences contained within the peptide of SEQ ID NO: 10 of US 2005/0070475 and likewise has been shown to exhibit anti-RSV activity, in particular, inhibiting fusion and syncytia formation between RSV-infected and uninfected Hep-2 cells at concentrations of less than 50 μg/ml.

### Anti-HPIV Peptides

Anti-HPIV peptides include DP178 and/or DP107 analogs identified from corresponding peptide sequences in HPIV and which have further been identified to inhibit viral infection by HPIV. Such peptides of interest include the peptides of Table 17 and SEQ ID NO:31 to SEQ ID NO:62 of US 2005/0070475. Detailed protocols for synthesizing these peptides are disclosed in US 2005/0070475, the contents of which are hereby specifically incorporated by reference. Of particular interest are the following peptides:

| | |
|---|---|
| VEAKQARSDIEKLKEAIRDTNKAVQSVQSSIGNLI | (SEQ ID NO:25) |
| RSDIEKLKEAIRDTNKAVQSVQSSIGNLIVAIKSV | (SEQ ID NO:26) |
| NSVALDPIDISIELNKAKSDLEESKEWIRRSNQKL | (SEQ ID NO:27) |
| ALDPIDISIELNKAKSDLEESKEWIRRSNQKLDSI | (SEQ ID NO:28) |
| LDPIDISIELNKAKSDLEESKEWIRRSNQKLDSIG | (SEQ ID NO:29) |
| DPIDISIELNKAKSDLEESKEWIRRSNQKLDSIGN | (SEQ ID NO:30) |
| PIDISIELNKAKSDLEESKEWIRRSNQKLDSIGNW | (SEQ ID NO:31) |
| IDISIELNKAKSDLEESKEWIRRSNQKLDSIGNWH | (SEQ ID NO:32) |

The peptide of SEQ ID NO:31 of US 2005/0070475 is derived from the F1 region of HPIV-3 and was identified in U.S. Pat. Nos. 6,103,236 and 6,020,459 using the search motifs described as corresponding to DP107 (i.e., “DP107-like”). The peptides of SEQ ID NO:25 and SEQ ID NO:26 each have amino acid sequences contained within the peptide of SEQ ID NO:30 of US 2005/0070475 and each has been shown to exhibit anti-HPIV-3 activity, in particular, inhibiting fusion and syncytia formation between HPIV-3-infected Hep2 cells and uninfected CV-1W cells at concentrations of less than 1 μg/ml.

The peptide of SEQ ID NO:32 of US 2005/0070475 is also derived from the F1 region of HPIV-3 and was identified in U.S. Pat. Nos. 6,103,236 and 6,020,459 using the search motifs described as corresponding to DP178 (i.e., “DP178-like”). The peptides of SEQ ID NO:27 and SEQ ID NO:28 to SEQ ID NO:32 each have amino acid sequences contained within the peptide of SEQ ID NO:32 of US 2005/0070475 and each also has been shown to exhibit anti-HPIV-3 activity, in particular, inhibiting fusion and syncytia formation between HPIV-3-infected Hep2 cells and uninfected CV-1W cells at concentrations of less than 1 μg/ml.

### Anti-MeV Peptides

Anti-MeV peptides are DP178 and/or DP107 analogs identified from corresponding peptide sequences in measles virus (MeV) which have further been identified to inhibit viral infection by the measles virus. Such peptides of particular interest include the peptides of Table 19 and peptides of SEQ ID NO:74 to SEQ ID NO:86 of US 2005/0070475. Detailed protocols for synthesizing these peptides are disclosed in US 2005/0070475, the contents of which are hereby specifically incorporated by reference. Of particular interest are the peptides listed below.

| | |
|---|---|
| HRIDLGPPISLERLDVGTNLGNAIAKLEAKELLE | (SEQ ID NO:33) |
| IDLGPPISLERLDVGTNLGNAIAKLEAKELLESS | (SEQ ID NO:34) |
| LGPPISLERLDVGTNLGNAIAKLEAKELLESSDQ | (SEQ ID NO:35) |
| PISLERLDVGTNLGNAIAKLEAKELLESSDQILR | (SEQ ID NO:36) |

Sequences derived from measles virus were identified in U.S. Pat. Nos. 6,103,236 and 6,020,459 using the search motifs described as corresponding to DP178 (i.e., “DP178-like”). The peptides of SEQ ID NO:33, SEQ ID NO:34, SEQ ID NO:35 and SEQ ID NO:36 each have amino acid sequences so identified, and each has been shown to exhibit anti-MeV activity, in particular, inhibiting fusion and syncytia formation between MeV-infected Hep2 and uninfected Vero cells at concentrations of less than 1 μg/ml.

### Anti-SIV Peptides

Anti-SIV peptides are DP178 and/or DP107 analogs identified from corresponding peptide sequences in SIV which have further been identified to inhibit viral infection by SIV. Such peptides of interest include the peptides of Table 18 and peptides of SEQ ID NO:63 to SEQ ID NO:73 of US 2005/0070475. Detailed protocols for synthesizing these peptides are disclosed in US 2005/0070475, the contents of which are hereby specifically incorporated by reference. Of particular interest are the following peptides:

| | |
|---|---|
| WQEWERKVDFLEENITALLEEAQIQQEKNMYELQK | (SEQ ID NO:37) |
| QEWERKVDFLEENITALLEEAQIQQEKNMYELQKL | (SEQ ID NO:38) |
| EWERKVDFLEENITALLEEAQIQQEKNMYELQKLN | (SEQ ID NO:39) |
| WERKVDFLEENITALLEEAQIQQEKNMYELQKLNS | (SEQ ID NO:40) |
| ERKVDFLEENITALLEEAQIQQEKNMYELQKLNSW | (SEQ ID NO:41) |
| RKVDFLEENITALLEEAQIQQEKNMYELQKLNSWD | (SEQ ID NO:42) |
| KVDFLEENITALLEEAQIQQEKNMYELQKLNSWDV | (SEQ ID NO:43) |
| VDFLEENITALLEEAQIQQEKNMYELQKLNSWDVF | (SEQ ID NO:44) |
| DFLEENITALLEEAQIQQEKNMYELQKLNSWDVFG | (SEQ ID NO:45) |
| FLEENITALLEEAQIQQEKNMYELQKLNSWDVFGN | (SEQ ID NO:46) |

Sequences derived from SIV transmembrane fusion protein were identified in U.S. Pat. Nos. 6,103,236 and 6,020,459 using the search motifs described as corresponding to DP178 (i.e., “DP178-like”). The peptides of SEQ ID NO:37 to SEQ ID NO:46 each have amino acid sequences so identified, and each has been shown to exhibit potent anti-SIV activity as crude peptides.

### Additional Viral and Fusion Inhibitors

The expression “viral inhibitor derivative” is intended to mean any modification or derivative of a viral inhibitor chosen from an antifusogenic compound or an entry Inhibitor (or non-antifusogenic) compound.

Antifusogenic compounds include, without limitation, enfuvirtide; C34; T-1249; TRI-899; TRI-999; 5-helix; N36 Mut (e.g); NCCG-gp41; DP-107; M41-P; N36; M87o; FM-006; ADS-J1; C14 linkmid; C34coil; hemolysin A; IQN17; IQN23; SC34EK; SPI-30,014; SPI-70,038; T-1249-HSA; T-649; T-651; TRI-1144; C14; MBP-107; scC34; SJ-2176; T-1249-transferrin; p26; p38; ADS-J2; C52L; clone 3 antibody; D5 IgG; D5 scFc; F240 scFv; sifuvirtide; IZN-36; T-1249 mimetibody; N-36-E; NB-2; NB-64; S-29-I; theaflavin-3,3′-digallate; VIRIP; siamycin I; siamycin II.

Entry Inhibitor (or non-antifusogenic) compounds include, without limitation, AMD-070; SPC-3; KRH-2731; AMD-8664; FC-131; HIV-1 Tat analogs; KRH-1120; KRH-1636; POL-2438; T-134; T-140; stromal cell-derived factor 1; ALX40-4C; AMD-3100; T-22; TJN-151; AM-1401; EradicAide viral macrophage inflammatory protein II; AMD-3451; conocurvone; maraviroc; vicriviroc; INCB-9471; INCB-15,050; DAPTA; PRO-140; HGS-004; SCH-C; TAK-652; TAK-220; nifeviroc; AMD-887; anti-CD63 MAb; AOP-RANTES; CPMD-167; E-913; FLSC R/T-IgG1; HGS-101; NIBR-1282; nonakine; PSC-RANTES; sCD4-17b; SCH-350,634; MIP-1 alpha; MIP-1 beta; RANTES; aplaviroc; peptide T; TAK-779; pCLXSN vector; UCB-35,625; J-113,863; CLIV; I-309; EGCG; Epigallocathechin Gallate; HB-19; lambda-carrageenan; PC-515; curdlan sulfate; OKU-40; OKU-41; VGV-1; Zintevir; AR-177; T-30,177; succinylated albumin; NSC0-658,586; ISIS-5320; RP-400c; SA-1042; C31G; Savvy; PRO-542; rCD4-IgG2; BMS-488,043; BMS-378,806; DES-6; 12 p1; Actinohivin; BlockAide/VP; CD4M33; CT-319; CT-326; cyanovirin-N; DCM-205; DES-10; griffithsin; HNG-105; NBD-556; NBD-557; PEG-cyanovirin-N; scytovirin; sCD4; dextrin-2-sulfate; F-105; FP-21,399; TNX-355; B4 MAb; R-15-K; sCD38(51-75) MBP; PRO-2000; NSC-13,778; SB-673,461M; SB-673,462M; rsCD4; Ac(Ala10,11) RANTES (2-14); IC-9564; RPR-103,611; Immudel-gp120; suligovir; IQP-0410; acetylated triiodothyronine; SP-01A; DEB-025; CSA-54; HGS-H/A 27; SP-10; VIR-5103; BMS-433,771; TMC-353,121; NSC-650,898; Michellamine B; NSC-692,906; TG-102; VIR-576; MEDI-488; CovX-Body; CNI-H0294.

### Modification of Anti-Viral and Antifusogenic Peptides

The invention contemplates modifying peptides that exhibit anti-viral and/or antifusogenic activity, including such modifications of DP-107 and DP-178 and analogs thereof. Such modified peptides can react with the available reactive functionalities on blood components via covalent linkages. The invention also relates to such modifications, such combinations with blood components, and methods for their use. These methods include extending the effective therapeutic life of the conjugated anti-viral peptides derivatives as compared to administration of the unconjugated peptides to a patient. The modified peptides are of a type designated as a DAC™ (Drug Affinity Complex) which comprises the anti-viral peptide molecule and a linking group together with a chemically reactive group capable of reaction with a reactive functionality of a mobile blood protein. By reaction with the blood component or protein the modified peptide, or DAC, may be delivered via the blood to appropriate sites or receptors.

To form covalent bonds with functionalities on the protein, one may use as a reactive group a wide variety of active carboxyl groups, particularly esters, where the hydroxyl moiety is physiologically acceptable at the levels required to modify the peptide. While a number of different hydroxyl groups may be employed in these reactive groups, the most convenient would be N-hydroxysuccinimide or (NHS), N-hydroxy-sulfosuccinimide (sulfo-NHS). In preferred embodiments of this invention, the functionality on the protein will be a thiol group and the reactive group will be a maleimido-containing group such as gamma-maleimide-butyralamide (GMBA) or maleimidopropionic acid (MPA).

Primary amines are the principal targets for NHS esters. Accessible α-amine groups present on the N-termini of proteins react with NHS esters. However, α-amino groups on a protein may not be desirable or available for the NHS coupling. While five amino acids have nitrogen in their side chains, only the ε-amine of lysine reacts significantly with NHS esters. An amide bond is formed when the NHS ester conjugation reaction reacts with primary amines releasing N-hydroxysuccinimide as demonstrated in the schematic below.

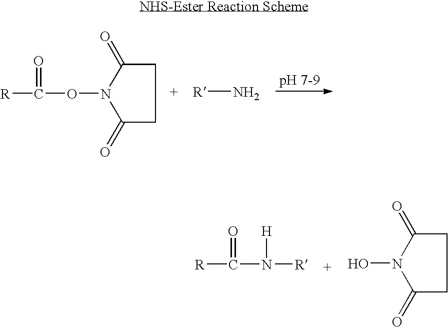

In the preferred embodiments of this invention, the functional group on this protein will be a thiol group and the chemically reactive group will be a maleimido-containing group such as MPA or GMBA (gamma-maleimide-butyralamide). The maleimido group is most selective for sulfhydryl groups on peptides when the pH of the reaction mixture is kept between 6.5 and 7.4. At pH 7.0, the rate of reaction of maleimido groups with sulfhydryls is 1000-fold faster than with amines. A stable thioether linkage between the maleimido group and the sulfhydryl is formed which cannot be cleaved under physiological conditions, as demonstrated in the following schematic.

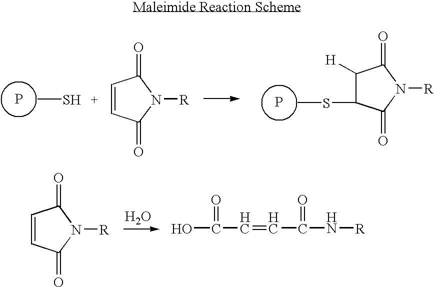

### Specific Labeling.

Preferably, the modified peptides of this invention are designed to specifically react with thiol groups on mobile blood proteins. Such reaction is preferably established by covalent bonding of the peptide modified with a maleimide link (e.g. prepared from GMBS, MPA or other maleimides) to a thiol group on a mobile blood protein such as serum albumin or IgG.

Under certain circumstances, specific labeling with maleimides offers several advantages over non-specific labeling of mobile proteins with groups such as NHS and sulfo-NHS. Thiol groups are less abundant in vivo than amino groups. Therefore, the maleimide-modified peptides of this invention, i.e., maleimide peptides, will covalently bond to fewer proteins. For example, in albumin (the most abundant blood protein) there is only a single thiol group. Thus, peptide-maleimide-albumin conjugates will tend to comprise approximately a 1:1 molar ratio of peptide to albumin. In addition to albumin, IgG molecules (class II) also have free thiols. Since IgG molecules and serum albumin make up the majority of the soluble protein in blood they also make up the majority of the free thiol groups in blood that are available to covalently bond to maleimide-modified peptides.

Further, even among free thiol-containing blood proteins, including IgGs, specific labeling with maleimides leads to the preferential formation of peptide-maleimide-albumin conjugates, due to the unique characteristics of albumin itself. The single free thiol group of albumin, highly conserved among species, is located at amino acid residue 34 (Cys³⁴). It has been demonstrated recently that the Cys³⁴ of albumin has increased reactivity relative to free thiols on other free thiol-containing proteins. This is due in part to the very low pK value of 5.5 for the Cys³⁴ of albumin. This is much lower than typical pK values for cysteine residues in general, which are typically about 8. Due to this low pK, under normal physiological conditions Cys³⁴ of albumin is predominantly in the ionized form, which dramatically increases its reactivity. In addition to the low pK value of Cys³⁴, another factor which enhances the reactivity of Cys³⁴ is its location, which is in a crevice close to the surface of one loop of region V of albumin. This location makes Cys³⁴ very available to ligands of all kinds, and is an important factor in Cys³⁴'s biological role as free radical trap and free thiol scavenger. These properties make Cys³⁴ highly reactive with maleimide-peptides, and the reaction rate acceleration can be as much as 1000-fold relative to rates of reaction of maleimide-peptides with other free-thiol containing proteins.

Another advantage of peptide-maleimide-albumin conjugates is the reproducibility associated with the 1:1 loading of peptide to albumin specifically at Cys³⁴. Other techniques, such as glutaraldehyde, DCC, EDC and other chemical activations of, e.g, free amines, lack this selectivity. For example, albumin contains 52 lysine residues, 25-30 of which are located on the surface of albumin and therefore accessible for conjugation. Activating these lysine residues, or alternatively modifying peptides to couple through these lysine residues, results in a heterogenous population of conjugates. Even if 1:1 molar ratios of peptide to albumin are employed, the yield will consist of multiple conjugation products, some containing 0, 1, 2 or more peptides per albumin, and each having peptides randomly coupled at any one or more of the 25-30 available lysine sites. Given the numerous possible combinations, characterization of the exact composition and nature of each conjugate batch becomes difficult, and batch-to-batch reproducibility is all but impossible, making such conjugates less desirable as a therapeutic. Additionally, while it would seem that conjugation through lysine residues of albumin would at least have the advantage of delivering more therapeutic agent per albumin molecule, studies have shown that a 1:1 ratio of therapeutic agent to albumin is preferred. In an article by Stehle, et al., “The Loading Rate Determines Tumor Targeting properties of Methotrexate-Albumin Conjugates in Rats,” Anti-Cancer Drugs, Vol. 8, pp. 677-685 (1988), the authors report that a 1:1 ratio of the anti-cancer methotrexate to albumin conjugated via glutaraldehyde gave the most promising results. These conjugates were preferentially taken up by tumor cells, whereas conjugates bearing 5:1 to 20:1 methotrexate molecules had altered HPLC profiles and were quickly taken up by the liver in vivo. It is postulated that at these higher ratios, conformational changes to albumin diminish its effectiveness as a therapeutic carier.

Through controlled administration of maleimide-peptides in vivo, one can control the specific labeling of albumin and IgG in vivo. In typical administrations, 80-90% of the administered maleimide-peptides will label albumin and less than 5% will label IgG. Trace labeling of free thiols such as glutathione will also occur. Such specific labeling is preferred for in vivo use as it permits an accurate calculation of the estimated half-life of the administered agent.

In addition to providing controlled specific in vivo labeling, maleimide-peptides can provide specific labeling of serum albumin and IgG ex vivo. Such ex vivo labeling involves the addition of maleimide-peptides to blood, serum or saline solution containing serum albumin and/or IgG. Once conjugation has occurred ex vivo with the maleimide-peptides, the blood, serum or saline solution can be readministered to the patient's blood for in vivo treatment.

In contrast to NHS-peptides, maleimide-peptides are generally quite stable in the presence of aqueous solutions and in the presence of free amines. Since maleimide-peptides will only react with free thiols, protective groups are generally not necessary to prevent the maleimide-peptides from reacting with itself. In addition, the increased stability of the modified peptide permits the use of further purification steps such as HPLC to prepare highly purified products suitable for in vivo use. Lastly, the increased chemical stability provides a product with a longer shelf life.

### Non-Specific Labeling.

The anti-viral peptides of the invention may also be modified for non-specific labeling of blood components. Bonds to amino groups will also be employed, particularly with the formation of amide bonds for non-specific labeling. To form such bonds, one may use as a chemically reactive group a wide variety of active carboxyl groups, particularly esters, where the hydroxyl moiety is physiologically acceptable at the levels required. While a number of different hydroxyl groups may be employed in these linking agents, the most convenient would be N-hydroxysuccinimide (NHS) and N-hydroxy-sulfosuccinimide (sulfo-NHS).

Other linking agents which may be utilized are described in U.S. Pat. No. 5,612,034.

The various sites with which the chemically reactive group of the modified peptides may react in vivo include cells, particularly red blood cells (erythrocytes) and platelets, and proteins, such as immunoglobulins, including IgG and IgM, serum albumin, ferritin, steroid binding proteins, transferrin, thyroxin binding protein, α-2-macroglobulin, and the like. Those receptors with which the modified peptides react, which are not long-lived, will generally be eliminated from the human host within about three days. The proteins indicated above (including the proteins of the cells) will remain at least three days, and may remain five days or more (usually not exceeding 60 days, more usually not exceeding 30 days) particularly as to the half life, based on the concentration in the blood.

For the most part, reaction will be with mobile components in the blood, particularly blood proteins and cells, more particularly blood proteins and erythrocytes. By “mobile” is intended that the component does not have a fixed situs for any extended period of time, generally not exceeding 5 minutes, more usually one minute, although some of the blood component may be relatively stationary for extended periods of time. Initially, there will be a relatively heterogeneous population of functionalized proteins and cells. However, for the most part, the population within a few days will vary substantially from the initial population, depending upon the half-life of the functionalized proteins in the blood stream. Therefore, usually within about three days or more, IgG will become the predominant functionalized protein in the blood stream.

Usually, by day 5 post-administration, IgG, serum albumin and erythrocytes will be at least about 60 mole %, usually at least about 75 mole %, of the conjugated components in blood, with IgG, IgM (to a substantially lesser extent) and serum albumin being at least about 50 mole %, usually at least about 75 mole %, more usually at least about 80 mole %, of the non-cellular conjugated components.

The desired conjugates of non-specific modified peptides to blood components may be prepared in vivo by administration of the modified peptides to the patient, which may be a human or other mammal. The administration may be done in the form of a bolus or introduced slowly over time by infusion using metered flow or the like.

If desired, the subject conjugates may also be prepared ex vivo by combining blood with modified peptides of the present invention, allowing covalent bonding of the modified peptides to reactive functionalities on blood components and then returning or administering the conjugated blood to the host. Moreover, the above may also be accomplished by first purifying an individual blood component or limited number of components, such as red blood cells, immunoglobulins, serum albumin, or the like, and combining the component or components ex vivo with the chemically reactive modified peptides. The functionalized blood or blood component may then be returned to the host to provide in vivo the subject therapeutically effective conjugates. The blood also may be treated to prevent coagulation during handling ex vivo.

### Synthesis of Modified Anti-Viral and Anti-Fusogenic Peptides

### A. Peptide Synthesis

Anti-viral and/or anti-fusogenic peptides according to the present invention may be synthesized by standard methods of solid phase peptide chemistry known to those of ordinary skill in the art. For example, peptides may be synthesized by solid phase chemistry techniques following the procedures described by Steward and Young (Steward, J. M. and Young, J. D., Solid Phase Peptide Synthesis, 2nd Ed., Pierce Chemical Company, Rockford, Ill., (1984) using an Applied Biosystem synthesizer. Similarly, multiple peptide fragments may be synthesized then linked together to form larger peptides. These synthetic peptides can also be made with amino acid substitutions at specific locations.

For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, Vol. 1, Acacemic Press (New York). In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid is then either attached to an inert solid support or utilized in solution by adding the next amino acid in the sequence having the complimentary (amino or carboxyl) group suitably protected and under conditions suitable for forming the amide linkage. The protecting group is then removed from this newly added amino acid residue and the next amino acid (suitably protected) is added, and so forth.

After all the desired amino acids have been linked in the proper sequence, any remaining protecting groups (and any solid support) are removed sequentially or concurrently to afford the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide.

A particularly preferred method of preparing compounds of the present invention involves solid phase peptide synthesis wherein the amino acid .alpha.-N-terminal is protected by an acid or base sensitive group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, alpha., .alpha.-dimethyl-3,5-dimethoxybenzyloxycarbonyl, o-nitrophenylsulfenyl, 2-cyano-t-butyloxycarbonyl, and the like. The 9-fluorenyl-methyloxycarbonyl (Fmoc) protecting group is particularly preferred for the synthesis of the peptides of the present invention. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxycarbonyl, 2,6-dichlorobenzyl, isopropyl, t-butyl (t-Bu), cyclohexyl, cyclopenyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl(trityl).

In the solid phase peptide synthesis method, the .alpha.-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. The preferred solid support for synthesis of .alpha.-C-terminal carboxy peptides is 4-hydroxymethylphenoxymethyl-copol-y(styrene-1% divinylbenzene). The preferred solid support for .alpha.-C-terminal amide peptides is the 4-(2′,4′-dimethoxyphenyl-Fmoc-am-inomethyl)phenoxyacetamidoethyl resin available from Applied Biosystems (Foster City, Calif). The .alpha.-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetra-methyluronium-hexafluorophosphate (HBTU), with or without 4-dimethylaminopyridine (DMAP), 1-hydroxybenzotriazole (HOBT), benzotriazol-1-yloxy-tris(dimethylamino)phosphonium-hexafluorophosphate (BOP) or bis(2-oxo-3-oxazolidinyl)phosphine chloride (BOPCl), mediated coupling for from about 1 to about 24 hours at a temperature of between 10.degree. and 50.degree. C. in a solvent such as dichloromethane or DMF.

When the solid support is 4-(2′,4′-dimethoxyphenyl-Fmoc-aminomethyl-)phenoxy-acetamidoethyl resin, the Fmoc group is cleaved with a secondary amine, preferably piperidine, prior to coupling with the .alpha.-C-terminal amino acid as described above. The preferred method for coupling to the deprotected 4-(2′,4′-dimethoxyphenyl-Fmoc-aminomethyl-)phenoxy-acetamidoethyl resin is O-benzotriazol-1-yl-N,N,N′,N′-tetramethyl-uroniumhexafluoro-phosphate (HBTU, 1 equiv.) and 1-hydroxybenzotriazole (HOBT, 1 equiv.) in DMF. The coupling of successive protected amino acids can be carried out in an automatic polypeptide synthesizer as is well known in the art. In a preferred embodiment, the .alpha.-N-terminal amino acids of the growing peptide chain are protected with Fmoc. The removal of the Fmoc protecting group from the .alpha.-N-terminal side of the growing peptide is accomplished by treatment with a secondary amine, preferably piperidine. Each protected amino acid is then introduced in about 3-fold molar excess, and the coupling is preferably carried out in DMF. The coupling agent is normally O-benzotriazol-1-yl-N,N,N′,N′-tetrame-thyluroniumhexafluorophosphate (HBTU, 1 equiv.) and 1-hydroxybenzotriazole (HOBT, 1 equiv).

At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the .alpha.-C-terminal of the polypeptide is an alkylamide, the resin is cleaved by aminolysis with an alkylamine. Alternatively, the peptide may be removed by transesterification, e.g. with methanol, followed by aminolysis or by direct transamidation. The protected peptide may be purified at this point or taken to the next step directly. The removal of the side chain protecting groups is accomplished using the cleavage cocktail described above. The fully deprotected peptide is purified by a sequence of chromatographic steps employing any or all of the following types: ion exchange on a weakly basic resin (acetate form); hydrophobic adsorption chromatography on underivitized polystyrene-divinylbenzene (for example, Amberlite XAD); silica gel adsorption chromatography; ion exchange chromatography on carboxymethylcellulose; partition chromatography, e.g. on Sephadex G-25, LH-20 or countercurrent distribution; high performance liquid chromatography (HPLC), especially reverse-phase HPLC on octyl- or octadecylsilyl-silica bonded phase column packing. Molecular weights of these ITPs are determined using Fast Atom Bombardment (FAB) Mass Spectroscopy.

### N-Terminal Protective Groups

As discussed above, the term “N-protecting group” refers to those groups intended to protect the .alpha.-N-terminal of an amino acid or peptide or to otherwise protect the amino group of an amino acid or peptide against undesirable reactions during synthetic procedures. Commonly used N-protecting groups are disclosed in Greene, “Protective Groups In Organic Synthesis,” (John Wiley & Sons, New York (1981)), which is hereby incorporated by reference. Additionally, protecting groups can be used as pro-drugs which are readily cleaved in vivo, for example, by enzymatic hydrolysis, to release the biologically active parent. .alpha.-N-protecting groups comprise loweralkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl-, 1-(p-biphenylyl)-1-methylethoxycarbonyl, .alpha., .alpha.-dimethyl-3,5-di-methoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like.

### Carboxy Protective Groups

As discussed above, the term “carboxy protecting group” refers to a carboxylic acid protecting ester or amide group employed to block or protect the carboxylic acid functionality while the reactions involving other functional sites of the compound are performed. Carboxy protecting groups are disclosed in Greene, “Protective Groups in Organic Synthesis” pp. 152-186 (1981), which is hereby incorporated by reference. Additionally, a carboxy protecting group can be used as a pro-drug whereby the carboxy protecting group can be readily cleaved in vivo, for example by enzymatic hydrolysis, to release the biologically active parent. Such carboxy protecting groups are well known to those skilled in the art, having been extensively used in the protection of carboxyl groups in the penicillin and cephalosporin fields as described in U.S. Pat. Nos. 3,840,556 and 3,719,667, the disclosures of which are hereby incorporated herein by reference. Representative carboxy protecting groups are C₁-C₈ loweralkyl (e.g., methyl, ethyl or t-butyl and the like); arylalkyl such as phenethyl or benzyl and substituted derivatives thereof such as alkoxybenzyl or nitrobenzyl groups and the like; arylalkenyl such as phenylethenyl and the like; aryl and substituted derivatives thereof such as 5-indanyl and the like; dialkylaminoalkyl such as dimethylaminoethyl and the like); alkanoyloxyalkyl groups such as acetoxymethyl, butyryloxymethyl, valeryloxymethyl, isobutyryloxymethyl, isovaleryloxymethyl, 1-(propionyloxy)-1-ethyl, 1-(pivaloyloxyl)-1-ethyl, 1-methyl-1-(propionyloxy)-1-ethyl, pivaloyloxymethyl, propionyloxymethyl and the like; cycloalkanoyloxyalkyl groups such as cyclopropylcarbonyloxymethyl, cyclobutylcarbonyloxymethyl, cyclopentylcarbonyloxymethyl, cyclohexylcarbonyloxymethyl and the like; aroyloxyalkyl such as benzoyloxymethyl, benzoyloxyethyl and the like; arylalkylcarbonyloxyalkyl such as benzylcarbonyloxymethyl, 2-benzylcarbonyloxyethyl and the like; alkoxycarbonylalkyl or cycloalkyloxycarbonylalkyl such as methoxycarbonylmethyl, cyclohexyloxycarbonylmethyl, 1-methoxycarbonyl-1-ethyl and the like; alkoxycarbonyloxyalkyl or cycloalkyloxycarbonyloxyalkyl such as methoxycarbonyloxymethyl, t-butyloxycarbonyloxymethyl, 1-ethoxycarbonyloxy-1-ethyl, 1-cyclohexyloxycarbonyloxy-1-ethyl and the like; aryloxycarbonyloxyalkyl such as 2-(phenoxycarbonyloxy)ethyl, 2-(5-indanyloxycarbonyloxy)ethyl and the like; alkoxyalkylcarbonyloxyalky-1 such as 2-(1-methoxy-2-methylpropan-2-oyloxy)ethyl and like; arylalkyloxycarbonyloxyalkyl such as 2-(benzyloxycarbonyloxy)ethyl and the like; arylalkenyloxycarbonyloxyalkyl such as 2-(3-phenylpropen-2-ylox-ylcarbonyloxy)ethyl and the like; alkoxycarbonylaminoalkyl such as t-butyloxycarbonylaminomethyl and the like; alkylaminocarbonylaminoalkyl such as methylaminocarbonylaminomethyl and the like; alkanoylaminoalkyl such as acetylaminomethyl and the like; heterocycliccarbonyloxyalkyl such as 4-methylpiperazinylcarbonyloxymethyl and the like; dialkylaminocarbonylalkyl such as dimethylaminocarbonylmethyl, diethylaminocarbonylmethyl and the like; (5-(loweralkyl)-2-oxo-1,3-dioxol-en-4-yl)alkyl such as (5-t-butyl-2-oxo-1,3-dioxolen-4-yl)methyl and the like; and (5-phenyl-2-oxo-1,3-dioxolen-4-yl)alkyl such as (5-phenyl-2-oxo-1,3-dioxolen-4-yl)methyl and the like.

Representative amide carboxy protecting groups are aminocarbonyl and lower alkylaminocarbonyl groups.

Preferred carboxy-protected compounds of the invention are compounds wherein the protected carboxy group is a loweralkyl, cycloalkyl or arylalkyl ester, for example, methyl ester, ethyl ester, propyl ester, isopropyl ester, butyl ester, sec-butyl ester, isobutyl ester, amyl ester, isoamyl ester, octyl ester, cyclohexyl ester, phenylethyl ester and the like or an alkanoyloxyalkyl, cycloalkanoyloxyalkyl, aroyloxyalkyl or an arylalkylcarbonyloxyalkyl ester. Preferred amide carboxy protecting groups are loweralkylaminocarbonyl groups. For example, aspartic acid may be protected at the alpha-C-terminal by an acid labile group (e.g., t-butyl) and protected at the beta-C-terminal by a hydrogenation labile group (e.g., benzyl) then deprotected selectively during synthesis.

### Peptide Modification

The manner of producing the modified peptides of the present invention will vary widely, depending upon the nature of the various elements comprising the peptide. The synthetic procedures will be selected so as to be simple, provide for high yields, and allow for a highly purified stable product. Normally, the chemically reactive group will be created at the last stage of the synthesis, for example, with a carboxyl group, esterification to form an active ester. Specific methods for the production of modified peptides of the present invention are described below.

Specifically, the selected peptide is first assayed for anti-viral activity, and then is modified with the linking group only at either the N-terminus, C-terminus or interior of the peptide. The anti-viral activity of this modified peptide-linking group is then assayed. If the anti-viral activity is not reduced dramatically (i.e., reduced less than 10-fold), then the stability of the modified peptide-linking group is measured by its in vivo lifetime. If the stability is not improved to a desired level, then the peptide is modified at an alternative site, and the procedure is repeated until a desired level of anti-viral and stability is achieved.

More specifically, each peptide selected to undergo modification with a linker and a reactive entity group will be modified according to the following criteria: if a terminal carboxylic group is available on the peptide and is not critical for the retention of anti-viral activity, and no other sensitive functional group is present on the peptide, then the carboxylic acid will be chosen as attachment point for the linker-reactive group modification. If the terminal carboxylic group is involved in anti-viral activity, or if no carboxylic acids are available, then any other sensitive functional group not critical for the retention of anti-viral activity will be selected as the attachment point for the linker-reactive entity modification. If several sensitive functional groups are available on a a peptide, a combination of protecting groups will be used in such a way that after addition of the linker/reactive entity and deprotection of all the protected sensitive functional groups, retention of anti-viral activity is still obtained. If no sensitive functional groups are available on the peptide, or if a simpler modification route is desired, synthetic efforts will allow for a modification of the original peptide in such a way that retention of anti-viral is maintained. In this case the modification will occur at the opposite end of the peptide

An NHS derivative may be synthesized from a carboxylic acid in absence of other sensitive functional groups in the peptide. Specifically, such a peptide is reacted with N-hydroxysuccinimide in anhydrous CH₂Cl₂ and EDC, and the product is purified by chromatography or recrystallized from the appropriate solvent system to give the NHS derivative.

Alternatively, an NHS derivative may be synthesized from a peptide that contains an amino and/or thiol group and a carboxylic acid. When a free amino or thiol group is present in the molecule, it is preferable to protect these sensitive functional groups prior to perform the addition of the NHS derivative. For instance, if the molecule contains a free amino group, a transformation of the amine into aN Fmoc or preferably into a tBoc protected amine is necessary prior to perform the chemistry described above. The amine functionality will not be deprotected after preparation of the NHS derivative. Therefore this method applies only to a compound whose amine group is not required to be freed to induce the desired anti-viral effect. If the amino group needs to be freed to retain the original properties of the molecule, then another type of chemistry described below has to be performed.

In addition, an NHS derivative may be synthesized from a peptide containing an amino or a thiol group and no carboxylic acid. When the selected molecule contains no carboxylic acid, an array of bifunctional linkers can be used to convert the molecule into a reactive NHS derivative. For instance, ethylene glycol-bis(succinimydylsuccinate) (EGS) and triethylamine dissolved in DMF and added to the free amino containing molecule (with a ratio of 10:1 in favor of EGS) will produce the mono NHS derivative. To produce an NHS derivative from a thiol derivatized molecule, one can use N-[-maleimidobutyryloxy]succinimide ester (GMBS) and triethylamine in DMF. The maleimido group will react with the free thiol and the NHS derivative will be purified from the reaction mixture by chromatography on silica or by HPLC.

An NHS derivative may also be synthesized from a peptide containing multiple sensitive functional groups. Each case will have to be analyzed and solved in a different manner. However, thanks to the large array of protecting groups and bifunctional linkers that are commercially available, this invention is applicable to any peptide with preferably one chemical step only to modify the peptide (as described above) or two steps (as described above involving prior protection of a sensitive group) or three steps (protection, activation and deprotection). Under exceptional circumstances only, would multiple steps (beyond three steps) synthesis be required to transform a peptide into an active NHS or maleimide derivative.

A maleimide derivative may also be synthesized from a peptide containing a free amino group and a free carboxylic acid. To produce a maleimide derivative from a amino derivatized molecule, one can use N-[.gamma.-maleimidobutyryloxy]succinimide ester (GMBS) and triethylamine in DMF. The succinimide ester group will react with the free amino and the maleimide derivative will be purified from the reaction mixture by crystallization or by chromatography on silica or by HPLC.

Finally, a maleimide derivative may be synthesized from a peptide containing multiple other sensitive functional groups and no free carboxylic acids. When the selected molecule contains no carboxylic acid, an array of bifunctional crosslinking reagents can be used to convert the molecule into a reactive NHS derivative. For instance maleimidopropionic acid (MPA) can be coupled to the free amine to produce a maleimide derivative through reaction of the free amine with the carboxylic group of MPA using HBTU/HOBt/DIEA activation in DMF.

Many other commercially available heterobifunctional crosslinking reagents can alternatively be used when needed. A large number of bifunctional compounds are available for linking to entities. Illustrative reagents include: azidobenzoyl hydrazide, N-[4-(p-azidosalicylamino)butyl]-3′-[2′-pyridyldithio)propionamide), bis-sulfosuccinimidyl suberate, dimethyl adipimidate, disuccinimidyl tartrate, N-.gamma.-maleimidobutyryloxysuccinimide ester, N-hydroxy sulfosuccinimidyl-4-azidobenzoate, N-succinimidyl [4-azidophenyl]-1,3′-di-thiopropionate, N-succinimidyl [4-iodoacetyl]aminobenzoate, glutaraldehyde, and succinimidyl 4-[N-maleimidomethyl]cyclohexane-1-carbo-xylate.

### Uses of Modified Anti-Viral Peptides

Modified anti-viral peptides of the invention may be used as a therapeutic agent in the treatment of patients who are suffering from viral infection, and can be administered to patients according to the methods described below and other methods known in the art. Effective therapeutic dosages of the modified peptides may be determined through procedures well known by those in the art and will take into consideration any concerns over potential toxicity of the peptide.

The modified peptides can also be administered prophylactically to previously uninfected individuals. This can be advantageous in cases where an individual has been subjected to a high risk of exposure to a virus, as can occur when individual has been in contact with an infected individual where there is a high risk of viral transmission. This can be especially advantageous where there is known cure for the virus, such as the HIV virus. As a example, prophylactic administration of a modified anti-HIV peptide would be advantageous in a situation where a health care worker has been exposed to blood from an HIV-infected individual, or in other situations where an individual engaged in high-risk activities that potentially expose that individual to the HIV virus.

### Administration of Modified Anti-Viral and Anti-Fusogenic Peptides

Generally, the modified peptides will be administered in a physiologically acceptable medium, e.g. deionized water, phosphate buffered saline (PBS), saline, aqueous ethanol or other alcohol, plasma, proteinaceous solutions, mannitol, aqueous glucose, alcohol, vegetable oil, or the like. Other additives which may be included include buffers, where the media are generally buffered at a pH in the range of about 5 to 10, where the buffer will generally range in concentration from about 50 to 250 mM, salt, where the concentration of salt will generally range from about 5 to 500 mM, physiologically acceptable stabilizers, and the like. The compositions may be lyophilized for convenient storage and transport.

The subject modified peptides will for the most part be administered parenterally, such as intravenously (IV), intraarterially (IA), intramuscularly (IM), subcutaneously (SC), or the like. Administration may in appropriate situations be by transfusion. In some instances, where reaction of the functional group is relatively slow, administration may be oral, nasal, rectal, transdermal or aerosol, where the nature of the conjugate allows for transfer to the vascular system. Usually a single injection will be employed although more than one injection may be used, if desired. The modified peptides may be administered by any convenient means, including syringe, trocar, catheter, or the like.

In certain embodiments, the modified peptides will be administered by pulmonary means by methods known in the art. Techniques for deep lung delivery of aerosol dry powder forms of peptides or proteins are disclosed by Patton et al. (1997) Chemtech 27(12):34-38. Additional references disclosing pulmonary administration of peptides include Senior, K. et al. (2000) PSTT Vol. 3:281-282; Gumbleton, M. (2006) Advanced Drug Delivery Reviews 58:993-995; Newhouse, M. T. (2006) Encyclopedia of Pharmaceutical Technology, entitled “Drug Delivery: Pulmonary Delivery;” and Labiris, N. R. (2003) J. Clin. Pharmacology 56:600-612. The contents of all of these references are hereby incorporated.

The particular manner of administration will vary depending upon the amount to be administered, whether a single bolus or continuous administration, or the like. Preferably, the administration will be intravascularly, where the site of introduction is not critical to this invention, preferably at a site where there is rapid blood flow, e.g., intravenously, peripheral or central vein. Other routes may find use where the administration is coupled with slow release techniques or a protective matrix. The intent is that the modified peptide be effectively distributed in the blood, so as to be able to react with the blood components. The concentration of the conjugate will vary widely, generally ranging from about 1 pg/ml to 50 mg/ml. The total administered intravascularly will generally be in the range of about 0.1 mg/ml to about 50 mg/ml, about 5 mg/ml to 40 mg/ml, about 10 to 30 mg/ml, about 10 to 20 mg/ml, or about 5 to 15 mg/ml, about 1 mg/ml to about 10 mg/ml, or about 1 to 5 mg/ml.

By bonding to long-lived components of the blood, such as immunoglobulin, serum albumin, red blood cells and platelets, a number of advantages ensue. The activity of the peptide is extended for days to weeks. Only one administration need be given during this period of time. Greater specificity can be achieved, since the active compound will be primarily bound to large molecules, where it is less likely to be taken up intracellularly to interfere with other physiological processes.

### Monitoring the Presence of Modified Peptides

The blood of the mammalian host may be monitored for the presence of the modified peptide compound one or more times. By taking a portion or sample of the blood of the host, one may determine whether the peptide has become bound to the long-lived blood components in sufficient amount to be therapeutically active and, thereafter, the level of the peptide compound in the blood. If desired, one may also determine to which of the blood components the peptide is bound. This is particularly important when using non-specific modified peptides. For specific maleimide-modified peptides, it is much simpler to calculate the half life of serum albumin and IgG.

### Immuno Assays

Another aspect of this invention relates to methods for determining the concentration of the anti-viral peptides and/or analogs, or their derivatives and conjugates in biological samples (such as blood) using antibodies specific for the peptides, peptide analogs or their derivatives and conjugates, and to the use of such antibodies as a treatment for toxicity potentially associated with such peptides, analogs, and/or their derivatives or conjugates. This is advantageous because the increased stability and life of the peptides in vivo in the patient might lead to novel problems during treatment, including increased possibility for toxicity.

The use of anti-therapeutic agent antibodies, either monoclonal or polyclonal, having specificity for a particular peptide, peptide analog or derivative thereof, can assist in mediating any such problem. The antibody may be generated or derived from a host immunized with the particular peptide, analog or derivative thereof, or with an immunogenic fragment of the agent, or a synthesized immunogen corresponding to an antigenic determinant of the agent. Preferred antibodies will have high specificity and affinity for native, modified and conjugated forms of the peptide, peptide analog or derivative. Such antibodies can also be labeled with enzymes, fluorochromes, or radiolables.

Antibodies specific for modified peptides may be produced by using purified peptides for the induction of peptide-specific antibodies. By induction of antibodies, it is intended not only the stimulation of an immune response by injection into animals, but analogous steps in the production of synthetic antibodies or other specific binding molecules such as screening of recombinant immunoglobulin libraries. Both monoclonal and polyclonal antibodies can be produced by procedures well known in the art.

The anti-peptide antibodies may be used to treat toxicity induced by administration of the modified peptide, analog or derivative thereof, and may be used ex vivo or in vivo. Ex vivo methods would include immuno-dialysis treatment for toxicity employing anti-therapeutic agent antibodies fixed to solid supports. In vivo methods include administration of anti-therapeutic agent antibodies in amounts effective to induce clearance of antibody-agent complexes.

The antibodies may be used to remove the modified peptides, analogs or derivatives thereof, and conjugates thereof, from a patient's blood ex vivo by contacting the blood with the antibodies under sterile conditions. For example, the antibodies can be fixed or otherwise immobilized on a column matrix and the patient's blood can be removed from the patient and passed over the matrix. The modified peptide, peptide analogs, derivatives or conjugates will bind to the antibodies and the blood containing a low concentration of peptide, analog, derivative or conjugate, then may be returned to the patient's circulatory system. The amount of peptide compound removed can be controlled by adjusting the pressure and flow rate.

Preferential removal of the peptides, analogs, derivatives and conjugates from the plasma component of a patient's blood can be effected, for example, by the use of a semipermeable membrane, or by otherwise first separating the plasma component from the cellular component by ways known in the art prior to passing the plasma component over a matrix containing the anti-therapeutic antibodies. Alternatively the preferential removal of peptide-conjugated blood cells, including red blood cells, can be effected by collecting and concentrating the blood cells in the patient's blood and contacting those cells with fixed anti-therapeutic antibodies to the exclusion of the serum component of the patient's blood.

The anti-therapeutic antibodies can be administered in vivo, parenterally, to a patient that has received the peptide, analogs, derivatives or conjugates for treatment. The antibodies will bind peptide compounds and conjugates. Once bound the peptide activity will be hindered if not completely blocked thereby reducing the biologically effective concentration of peptide compound in the patient's bloodstream and minimizing harmful side effects. In addition, the bound antibody-peptide complex will facilitate clearance of the peptide compounds and conjugates from the patient's blood stream.

The invention having been fully described can be further appreciated and understood with reference to the following non-limiting examples.

### EXAMPLES

### Example 1-5

### Synthesis and Purification of Cysteic Acid Derivatives of C34

Synthesis of cysteic acid derivatives of C34 is performed using an automated solid-phase procedure on a Symphony Peptide Synthesizer with manual intervention during the generation of the peptide. The synthesis was performed on Fmoc-protected Ramage amide linker resin, using Fmoc-protected amino acids. Coupling was achieved by using O-benzotriazol-1-yl-N,N,N′,N′-tetramethyl-uronium hexafluorophosphate (HBTU) and diisopropylethylamine (DIEA) as the activator cocktail in N,N-dimethylformamide (DMF) solution. The Fmoc protective group was removed using 20% piperidine/DMF. A Boc-protected amino acid was used at the N-terminus in order to generated the free N_{α}-terminus once the peptides were cleaved from the resin. Sigmacoted glass reaction vessels were used during the synthesis.

### Example 2

### Synthesis Of CA-C34

CA-C34 has the following amino acid sequence:

Cysteic Acid-Trp-Met-Glu-Trp-Asp-Arg-Glu-Ile-Asn-Asn-Tyr-Thr-Ser-Leu-Ile-His-Ser-Leu-Ile-Glu-Glu-Ser-Gln-Asn-Gln-Gln-Glu-Lys-Asn-Glu-Gln-Glu-Leu-Leu-CONH₂ (SEQ ID NO:2). The CA-C34 modified peptide was synthesized as follows:

Step 1: Solid phase peptide synthesis of CA-C34 on a 100 μmole scale was performed using manual and automated solid-phase synthesis, a Symphony Peptide Synthesizer and Ramage resin. The following protected amino acids were sequentially added to resin: Fmoc-Leu-OH, Fmoc-Leu-OH, Fmoc-Glu(tBu)-OH, Fmoc-Gln(Trt)-OH, Fmoc-Glu(tBu)-OH, Fmoc-Asn(Trt)-OH, Fmoc-Lys(Boc)-OH, Fmoc-Glu(tBu)-OH, Fmoc-Gln(Trt)-OH, Fmoc-Gln(Trt)-OH, Fmoc-Asn(Trt)-OH, Fmoc-Gln(Trt)-OH, Fmoc-Ser(tBu)-OH, Fmoc-Glu(tBu)-OH, Fmoc-Glu(tBu)-OH, Fmoc-Ile-OH, Fmoc-Leu-OH, Fmoc-Ser(tBu)-OH, Fmoc-His(Trt)-OH, Fmoc-Ile-OH, Fmoc-Leu-OH, Fmoc-Ser(tBu)—OH, Fmoc-Thr(tBu)-OH, Fmoc-Tyr(tBu)-OH, Fmoc-Asn(Trt)-OH, Fmoc-Asn(Trt)-OH, Fmoc-Ile-OH, Fmoc-Glu(tBu)-OH, Fmoc-Arg(Pbf)-OH, Fmoc-Asp(tBu)-OH, Fmoc-Trp(Boc)-OH, Fmoc-Glu(tBu)-OH, Fmoc-Met-OH, Fmoc-Trp(Boc)-OH, Boc-Cyteic Acid-OH. They were dissolved in N,N-dimethylformamide (DMF) and, according to the sequence, activated using O-benzotriazol-1-yl-N,N, N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold (0-4° C.) Et₂O and collection.

### Example 3

### Synthesis Of CA-C34 (Arg 28)

CA-C34 (Arg²⁸) has the following amino acid sequence:

(SEQ ID NO:3)

Cysteic Acid-Trp-Met-Glu-Trp-Asp-Arg-Glu-Ile-Asn-
Asn-Tyr-Thr-Ser-Leu-Ile-His-Ser-Leu-Ile-Glu-Glu-
Ser-Gln-Asn-Gln-Gln-Glu-Arg-Asn-Glu-Gln-Glu-Leu-
Leu-CONH₂.

Step 1: Solid phase peptide synthesis of CA-C34 (Arg²⁸) on a 100 μmole scale was performed using manual and automated solid-phase synthesis, a Symphony Peptide Synthesizer and Ramage resin. The following protected amino acids were sequentially added to resin: Fmoc-Leu-OH, Fmoc-Leu-OH, Fmoc-Glu(tBu)-OH, Fmoc-Gln(Trt)-OH, Fmoc-Glu(tBu)-OH, Fmoc-Asn(Trt)-OH, Fmoc-Arg(Pbf)-OH, Fmoc-Glu(tBu)—OH, Fmoc-Gln(Trt)-OH, Fmoc-Gln(Trt)-OH, Fmoc-Asn(Trt)-OH, Fmoc-Gln(Trt)-OH, Fmoc-Ser(tBu)-OH, Fmoc-Glu(tBu)-OH, Fmoc-Glu(tBu)-OH, Fmoc-Ile-OH, Fmoc-Leu-OH, Fmoc-Ser(tBu)-OH, Fmoc-His(Trt)-OH, Fmoc-Ile-OH, Fmoc-Leu-OH, Fmoc-Ser(tBu)-OH, Fmoc-Thr(tBu)-OH, Fmoc-Tyr(tBu)-OH, Fmoc-Asn(Trt)-OH, Fmoc-Asn(Trt)-OH, Fmoc-Ile-OH, Fmoc-Glu(tBu)-OH, Fmoc-Arg(Pbf)-OH, Fmoc-Asp(tBu)—OH, Fmoc-Trp(Boc)-OH, Fmoc-Glu(tBu)-OH, Fmoc-Met-OH, Fmoc-Trp(Boc)-OH, Boc-Cyteic Acid-OH. They were dissolved in N,N-dimethylformamide (DMF) and, according to the sequence, activated using O-benzotriazol-1-yl-N,N, N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold (0-4° C.) Et₂O and collection.

### Example 4

### Synthesis of CA-C34-Lys³⁵ (ε-AEEA-MPA)

CA-C34-Lys³⁵ (ε-AEEA-MPA) has the following amino acid sequence:

(SEQ ID NO:4)

Cysteic Acid-Trp-Met-Glu-Trp-Asp-Arg-Glu-Ile-Asn-
Asn-Tyr-Thr-Ser-Leu-Ile-His-Ser-Leu-Ile-Glu-Glu-
Ser-Gln-Asn-Gln-Gln-Glu-Lys-Asn-Glu-Gln-Glu-Leu-
Leu-Lys(AEEA-MPA)-CONH₂.

Step 1: Solid phase peptide synthesis of CA-C34-Lys³⁵ (ε-AEEA-MPA) on a 100 μmole scale was performed using manual and automated solid-phase synthesis, a Symphony Peptide Synthesizer and Ramage resin. The following protected amino acids were sequentially added to resin: Fmoc-Lys(Aloc)-OH, Fmoc-Leu-OH, Fmoc-Leu-OH, Fmoc-Glu(tBu)-OH, Fmoc-Gln(Trt)-OH, Fmoc-Glu(tBu)-OH, Fmoc-Asn(Trt)-OH, Fmoc-Lys(Boc)-OH, Fmoc-Glu(tBu)-OH, Fmoc-Gln(Trt)-OH, Fmoc-Gln(Trt)-OH, Fmoc-Asn(Trt)-OH, Fmoc-Gln(Trt)-OH, Fmoc-Ser(tBu)-OH, Fmoc-Glu(tBu)-OH, Fmoc-Glu(tBu)-OH, Fmoc-Ile-OH, Fmoc-Leu-OH, Fmoc-Ser(tBu)-OH, Fmoc-His(Trt)-OH, Fmoc-Ile-OH, Fmoc-Leu-OH, Fmoc-Ser(tBu)-OH, Fmoc-Thr(tBu)-OH, Fmoc-Tyr(tBu)-OH, Fmoc-Asn(Trt)-OH, Fmoc-Asn(Trt)-OH, Fmoc-Ile-OH, Fmoc-Glu(tBu)—OH, Fmoc-Arg(Pbf)-OH, Fmoc-Asp(tBu)-OH, Fmoc-Trp(Boc)-OH, Fmoc-Glu(tBu)-OH, Fmoc-Met-OH, Fmoc-Trp(Boc)-OH, Boc-Cyteic Acid-OH. They were dissolved in N,N-dimethylformamide (DMF) and, according to the sequence, activated using O-benzotriazol-1-yl-N,N, N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the Fmoc-AEEA-OH and 3-maleimidopropionic acid (Step 3). The Protecting group (Fmoc) on the AEEA was removed as previously describe and between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold (0-4° C.) Et₂O (Step 4) and collected.

### Example 5

### CA-C34 (Arg²⁸)-Lys³⁵ (ε-AEEA-MPA)

CA-C34 (Arg²⁸)-Lys³⁵ (ε-AEEA-MPA) has the following sequence:

(SEQ ID NO:5)

Cysteic Acid-Trp-Met-Glu-Trp-Asp-Arg-Glu-Ile-Asn-
Asn-Tyr-Thr-Ser-Leu-Ile-His-Ser-Leu-Ile-Glu-Glu-
Ser-Gln-Asn-Gln-Gln-Glu-Arg-Asn-Glu-Gln-Glu-Leu-
Leu-Lys(AEEA-MPA)-CONH₂.

Step 1: Solid phase peptide synthesis of CA-C34 (Arg²⁸)-Lys³⁵ (ε-AEEA-MPA) on a 100 μmole scale was performed using manual and automated solid-phase synthesis, a Symphony Peptide Synthesizer and Ramage resin. The following protected amino acids were sequentially added to resin: Fmoc-Lys(Aloc)-OH, Fmoc-Leu-OH, Fmoc-Leu-OH, Fmoc-Glu(tBu)-OH, Fmoc-Gln(Trt)-OH, Fmoc-Glu(tBu)-OH, Fmoc-Asn(Trt)-OH, Fmoc-Arg(Pbf)-OH, Fmoc-Glu(tBu)-OH, Fmoc-Gln(Trt)-OH, Fmoc-Gln(Trt)-OH, Fmoc-Asn(Trt)-OH, Fmoc-Gln(Trt)-OH, Fmoc-Ser(tBu)-OH, Fmoc-Glu(tBu)-OH, Fmoc-Glu(tBu)-OH, Fmoc-Ile-OH, Fmoc-Leu-OH, Fmoc-Ser(tBu)-OH, Fmoc-His(Trt)-OH, Fmoc-Ile-OH, Fmoc-Leu-OH, Fmoc-Ser(tBu)-OH, Fmoc-Thr(tBu)-OH, Fmoc-Tyr(tBu)-OH, Fmoc-Asn(Trt)-OH, Fmoc-Asn(Trt)-OH, Fmoc-Ile-OH, Fmoc-Glu(tBu)—OH, Fmoc-Arg(Pbf)-OH, Fmoc-Asp(tBu)-OH, Fmoc-Trp(Boc)-OH, Fmoc-Glu(tBu)-OH, Fmoc-Met-OH, Fmoc-Trp(Boc)-OH, Boc-Cyteic Acid-OH. They were dissolved in N,N-dimethylformamide (DMF) and, according to the sequence, activated using O-benzotriazol-1-yl-N,N, N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the Fmoc-AEEA-OH and 3-maleimidopropionic acid (Step 3). The Protecting group (Fmoc) on the AEEA was removed as previously describe and between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold (0-4° C.) Et₂O (Step 4) and collected.

### Purification Procedure:

Each C34 modified peptide was purified by preparative reversed phase HPLC, using a Varian (Dynamax) preparative binary HPLC system.

Purification of the exemplified derivatives was performed using a Phenomenex Luna 10μ phenyl-hexyl, 50 mm×250 mm column (particules 10μ) equilibrated with a water/TFA mixture (0.1% TFA in H₂O; Solvent A) and acetonitrile/TFA (0.1% TFA in CH₃CN; Solvent B). Elution was achieved at 50 mL/min by running a 28-38% B gradient over 180 min. Fractions containing peptide were detected by UV absorbance (Varian Dynamax UVD II) at 214 and 254 nm.

Fractions were collected in 25 mL aliquots. Fractions containing the desired product were identified by mass detection after direct injection onto LC/MS. The selected fractions were subsequently analyzed by analytical HPLC (20-60% B over 20 min; Phenomenex Luna 5μ phenyl-hexyl, 10 mm×250 mm column, 0.5 mL/min) to identify fractions with ≧90% purity for pooling. The pool was freeze-dried using liquid nitrogen and subsequently lyophilized for at least 2 days to yield a white powder.

IV-Flow Diagram:

Identical synthetic schemes were employed for the all derivatives. The schemes for CA-C34 and CA-C34-Lys³⁵ (s-AEEA-MPA) are exemplified in the flow diagram below. Of course, the Aloc removal step along with the addition of AEEA and MPA were omitted for CA-C34.

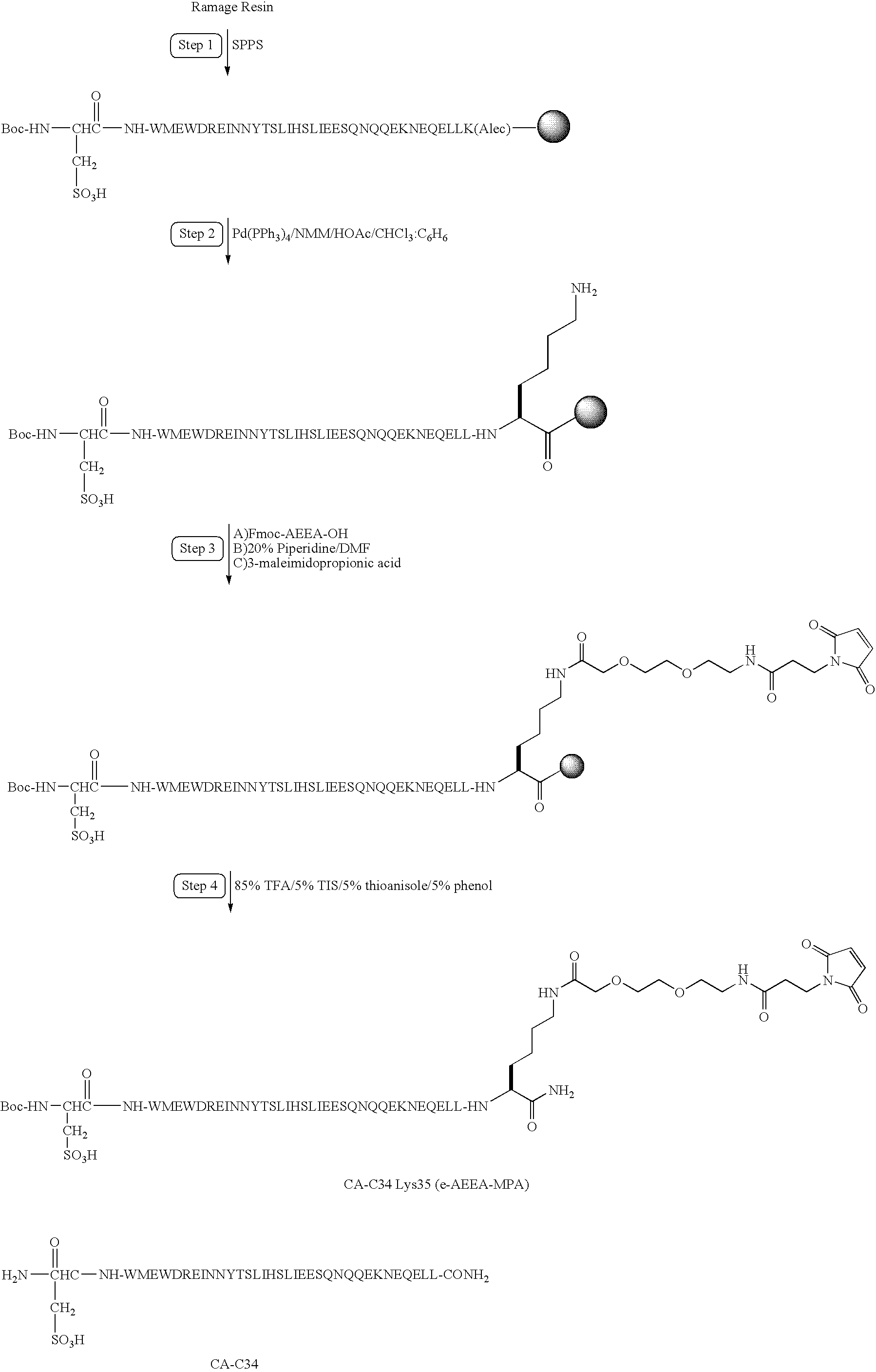

### Example 6

### Solubility Assays of Cysteic Acid Derivatives of C34

a. Solubility assays were performed in 100 mM sodium phosphate (starting pH 8) in water. It has been found that high buffer concentration is useful to neutralize the excess amounts of trifluoro acetic acid (TFA) that remains with the C34 derivatives after purification by HPLC.

Final pH of 5.8-7.0 is suitable for maintaining the solubility of the various C-34 derivatives. Therefore, the starting buffer is prepared at pH 8.0; and C34 derivatives solubilisation results in a final pH of approximately 6.3. Table 2 shows solubility limits of C34 with or without cysteic acid (CA) at the N-terminus, and with or without Lys³⁵(6-AEEA-MPA) in C-terminal. Furthermore, the final osmolalities shown in Table 1 reveal that these final solutions are isotonic.

TABLE 2

| | Solubility | | | |
|---|---|---|---|---|
| C34 and C34 | Limits ¹ | Final | Osmolality | |
| Derivatives | (mg/ml) | pH | (mOsm) | End Result |
| C34 | 15.75 | | | Gel formation |
| | | | | within minutes |
| C34-Lys³⁵(ε- | N/A ² | | | Gel formation |
| AEEA-MPA) | | | | |
| CA-C34 | 29.3 | 6.32 | 301 | Clear solution |
| CA-C34-Lys³⁵(ε- | 33.8 | 6.27 | 302 | Clear Yellowish ³ |
| AEEA-MPA) | | | | solution |

¹ Solubility limits indicated is the maximal concentration to maintain a clear solution. The concentration is corrected to represent the C34 derivatives weight free of TFA.
² “N/A” means the compound is not found to be soluble.
³ The yellowish color that is observed may be due to higher concentrations of (AEEA-MPA) or due to impurities.

Native C34 is to be found soluble at 15.75 mg/ml and the resulting solution forms a gel within a minute. C34-Lys³⁵ (ε-AEEA-MPA) forms a gel as soon as it is put in solution and further addition of buffer never succeed to solubilise the compound. As it can be noted from Table 1, addition of cysteic acid at the N-terminal end of both of these compounds confers significantly increased solubility to C34, i.e. 29.3 and 33.8 mg/ml, respectively.

b. Solubility of N-terminally modified AEEA-MPA linked to C34 (W1(AEEA-MPA)-C34) and N-terminal cysteic acid modified—C34 having a lysine addition at position 35 linked via an AEEA linker to MPA (CA K35(AEEA-MPA-C34)) at 30-35 mg/ml in 500 mM Sodium Phosphate buffer pH 8.0.

1 M Sodium Phosphate pH 8.0 buffer
A—2M sodium phosphate dibasic, anhydre, UQAM, OM-27, S1835 1M=141.96 g/1 L, 2 M=13.3568 g/40 ml nanopure H₂O.
B—2 M sodium phosphate, monobasic, monohydrate, UQAM, OM-27, S1820 1 M=137.99 g/1 L, 2M=11.0392 g/40 ml nanopure H₂O
C— Mix 30 ml nanopure H₂O+approximately 28 ml dibasic sodium phosphate+1.5 ml monobasic sodium phosphate. Verify if pH is at 8.0. Adjust volume with dibasic sodium phosphate.
D—Adjust for final pH 8 (real 7.98).
   500 mM Sodium Phosphate pH 8.0 buffer

Mixed 1 M sodium phosphate pH 8.0 buffer with an equal volume of nanopure H₂O (not filtered). Final pH is at 8.0.

c. Solubility of W1(AEEA-MPA)-C34 and (CA K35(AEEA-MPA-C34)) at 100 mg/ml in 500 mM Sodium Phosphate pH 8.0

### Compounds:

### W1 (AEEA-MPA)-C34 Batch B, lot no JC-205-12, Inventory no 1139.

1 M with salts=4769.1=12.64 mg weighed
1 M no salts=4541.1=12.0357 mg
Purity=90.8%=10.928 mg in 109.3 ul 500 mM Sodium Phosphate pH 8.0 for 100 mg/ml.
Comments: Buffer added to powder in glass vial. Soluble after 1 min. of vortex (medium speed). 3 particles left. Final pH 6.82 (measured with pH meter from Chemistry Department, 500 mM NaP pH 8 buffer was at 8.1).

### (CA K35(AEEA-MPA-C34)) Batch B, lot no PB-262-01, Inventory no 1352.

1 M with salts=5165.2=12.11 mg weighed
1 M no salts=4820.2=11.30 mg
Purity=92.8%=10.487 mg in 104.9 ul 500 mM Sodium Phosphate pH 8.0 for 100 mg/ml.

Comments: Buffer added to powder in glass vial. Mostly soluble after 1 min. 30 sec. of vortex (medium speed). 2 small pellets in the bottom of the glass vial. After 3 min., theses 2 small pellets were solubilized. Final pH 6.75 (measured with pH meter from Chemistry Department, 500 mM NaP pH 8 buffer was at 8.1).

W1(AEEA-MPA)-C34 and (CA K35(AEEA-MPA-C34)) are yellow once solubilized. W1(AEEA-MPA)-C34 is darker.

### Conclusion:

The W1(AEEA-MPA)-C34 and (CA K35(AEEA-MPA-C34)) compounds are soluble at 100 mg/ml in 500 mM sodium phosphate pH 8.0 buffer. Their final pH is above accepted limit i.e 6.8. The acceptable limit of pH for these compounds is 6.2.

d. Solubility of W1(AEEA-MPA)-C34 and (CA K35(AEEA-MPA-C34)) at 150 mg/ml in 500 mM Sodium Phosphate pH 8.0

### W1(AEEA-MPA)-C34 Batch B, lot no JC-205-12, Inventory no 1139.

1 M with salts=4769.1=11.97 mg weighed
1 M no salts=4541.1=11.398 mg
Purity=90.8%=10.35 mg in 69 ul 500 mM Sodium Phosphate pH 8.0 for 150 mg/ml.

Comments: Buffer added to powder in glass vial. Soluble after 1 min. of vortex (medium-fast speed). Final pH 6.60 (measured with pH meter from Chemistry Department, 500 mM NaP pH 8 buffer was at 8.23).

### (CA K35(AEEA-MPA-C34)) Batch B, lot no PB-262-01, Inventory no 1352.

1 M with salts=5165.2=12.48 mg weighed

1 M no salts=4820.2=11.64 mg

Purity=92.8%=10.808 mg in 72.05 ul 500 mM Sodium Phosphate pH 8.0 for 150 mg/mil.

Comments: Buffer added to powder in glass vial. Mostly soluble after 1 min. of vortex (medium-fast speed). 1 small pellet in the bottom of the glass vial. After 10 min., this small pellet was solubilized. Final pH 6.57 (measured with pH meter from Chemistry Department, 500 mM NaP pH 8 buffer was at 8.23).

W1(AEEA-MPA)-C34 and (CA K35(AEEA-MPA-C34)) are yellow once solubilized. W1(AEEA-MPA)-C34 is darker.

Conclusion: W1(AEEA-MPA)-C34 and (CA K35(AEEA-MPA-C34)) are both soluble at 150 mg/ml in 500 mM Sodium Phosphate pH 8.0 buffer.

### Example 7

### Activity Assays of Cysteic Acid Derivatives of C34

First Activity Assay: Anti-HIV-1-induced Cell-Cell Fusion Assay

The efficacy of various anti-fusogenic compounds and respective prefonmed albumin conjugates were evaluated using a HIV-1-induced cell-cell fusion assay designed by Dr. Shibo Jiang and co-workers at the New York Blood Center, 310 East 67^{th} Street, New York, N.Y. 10021. The inhibitory activity of C34 derivatives on HIV-induced cell-cell fusion was detected as previously described (Jiang, S. L. et al. (2000) A Convenient Cell Fusion Assay for Rapid Screening for HIV Entry Inhibitors, Proc. SPIE 3926: 212-219).

Briefly, a compound was first diluted in phosphate-citrate buffer (pH 7.0) at 100 μM as a stock solution, and then further diluted in culture medium at 1000, 500, 250, 100, 50, 25, 5, 1 nM. Fifty microlitres of the compound solution was mixed with 50 μl of HIV-1_{IIB} infected H9 cells (H9/HIV-1_{IIIB}) labeled with calcein-AM (Molecular Probes, Inc., Eugene, Oreg.) at 2×10⁵ cells/ml. After co-culture at 37° C. for 2 hrs, calcein-labeled H9/HIV-1_{IIIB} cells, both fused or unfused with MT-2 cells were counted under an inverted fluorescence microscope (Zeiss, Gennany) using filter excitation wavelengths of 485 nm and 535 nm, respectively, with an eyepiece micrometer disc (10×10 mm sq.) and a 20× objective. The fused cell is much larger (at least 2-fold) than the unfused cell, and thus, the intensity of fluorescence in the fused cell is weaker than that for the unfused cell due to the diffusion of calcein from one cell to two or more cells. Four fields per well were examined and the percentage of cell fusion was calculated by the following formula: fused cells/(fused+unfused cells)×100%.

The wells for positive control were added with 50 μl of calcein-labeled HIV-infected cells. The wells for negative controls were added with culture medium and calcein-labeled uninfected H9 cells. The percent inhibition of cell fusion was calculated using the following formula: [1−(E−N)/(P−N)]×100%, where “E” represents the % cell fusion in the experimental group, “P” represents the % fusion in the positive control group to which no test compound was added, “N” means the % fusion in the negative control group where calcein-labeled H9/HIV-1_{IIIB} cells were replaced by calcein-labeled H9 cells. The concentration for 50% inhibition (IC₅₀) of cell fusion by an antiviral compound was calculated using a computer program kindly provided by Dr. T. C. Chou (Chou, T. C. and Hayball, M. P., CalcuSyn: Windows software for dose effect analysis (1991) Ferguson, Mo. 63135, USA, BIOSOFT.

Table 3 shows anti-fusiogenic activity of C34 with and without a cysteic acid at the N-terminal; and with and without being conjugated to human serum albumin (HSA) via the group Lys(F-AEEA-MPA).

TABLE 3

| C34, C34 Derivatives and | |
|---|---|
| Albumin Conjugates thereof | IC50 (nM) |
| C34 | 3.6-4.6 |
| C34-Lys³⁵(ε-AEEA-MPA):HSA | 16.17 |
| CA-C34 | 7.3 |
| CA-C34-Lys³⁵(ε-AEEA-MPA):HSA | 6.1-9.4 |

As shown in Table 3, no significant difference is observed between the anti-fusiogenic activities of C34 and CA-C34. Therefore, the addition of cysteic acid at the N-terminal end of C34 does not negatively impact upon the anti-fusiogenic activity of C34.

Table 2 also shows that coupling Lys (ε-AEEA-MPA) to C34 and CA-C34 to their C-terminal end following by their conjugation to HSA, has no significant negative effect on their anti-fusiogenic activities.

### Example 8

### Second Activity Assay: Inhibition of HIV_{IIIb} Replication in Human PBMCS

The anti-HIV efficacy and cellular cytotoxicity of the compounds were assessed following acute infection in a PBMC based assay using the HIV-1 strain IIIB. These experiments were carried out at Southern Research Institute, Infectious Disease Research Department, 431 Aviation Way, Frederick, Md., following the protocol described below.

a. HIV-1 Infection of PBMCs

Fresh human PBMCs, seronegative for HIV and HBV, were isolated from screened donors and commercially provided by Biological Specialty Corporation Colmar, Pennsylvania. Cells were pelleted/washed 2-3 times by low speed centrifugation and re-suspension in PBS to remove contaminating platelets. The leukophoresed blood was then diluted with Dulbecco's Phosphate Buffered Saline (DPBS) and layered over Lymphocyte Separation Medium (LSM; Cellgro® by Mediatech, Inc.; density 1.078+/−0.002 g/ml; Cat. #85-072-CL) in a 50 mL centrifuge tube and then centrifuged. The buffy coat layer was gently aspirated from the resulting interface and subsequently washed with PBS by low speed centrifugation. After the third wash, cells were re-suspended in RPMI 1640 supplemented with fetal bovine serum (FBS), L-glutamine, penicillin, streptomycin, and phytohemagglutinin (PHA-P; Sigma, St-Louis, Mo). The cells were incubated at 37° C. After two days incubation, PBMCs were centrifuged and resuspended in RPMI 1640 with FBS, L-glutamine, penicillin, streptomycin, and recombinant human IL-2 (R&D Systems, Inc., Minneapolis, Minn). IL-2 is included in the culture medium to maintain the cell division initiated by the PHA mitogenic stimulation. Cells were kept in culture for a maximum of two weeks and monocytes were depleted from the culture as the result of adherence to the tissue culture flask.

For the standard PBMC assay, PHA-P stimulated cells from at least two normal donors were pooled, diluted in fresh media and plated in the interior wells of a 96 well round bottom microplate in a standard format developed by the Infectious Disease Research department of Southern Research Institute. Pooling PBMCs from more than one donor is used to minimize the variability observed between individual donors, which results from quantitative and qualitative differences in HIV infection and overall response to the PHA and IL-2 of primary lymphocyte populations. Each plate contains virus/cell control wells (cells+virus), experimental wells (compound+cells+virus) and compound control wells (compound+media, no cells, necessary for MTS monitoring of cytotoxicity). Test compound dilutions were prepared in microtiter tubes and each concentration was placed in appropriate wells using the standard format. Following addition of the compound dilutions to the PBMCs, a predetermined dilution of virus stock solution was then placed in each test well (final MOI≅0. 1). The virus stock solution is prepared from a low passage clinical isolate HIV-1_{IIIB} obtained from the NIAID AIDS Research and Reference Reagent Program. A pre-titered aliquot of HIV-1_{IIIB} stored at −80° C. was thawed rapidly to room temperature in a biological safety cabinet immediately before use. Since HIV-1 is not cytopathic to PBMCs, the same assay plate can be used for both antiviral efficacy and cytotoxicity measurements. The PBMC cultures were maintained for seven days following infection at 37° C., 5% CO₂.

b. Reverse Transcriptase Activity Assay

A microtiter plate-based reverse transcriptase (RT) reaction was utilized (Buckheit et al., AIDS Research and Human Retroviruses 7:295-302, 1991). Tritiated thymidine triphosphate (3H-TTP, 80 Ci/mmol, NEN) was received in 1:1 dH₂O:ethanol at 1 mCi/ml. Poly rA:oligo dT template:primer (Pharmacia) was prepared as a stock solution by combining 150 μl poly rA (20 mg/ml) with 0.5 ml oligo dT (20 units/ml) and 5.35 ml sterile dH₂O followed by aliquoting (1.0 ml) and storage at −20° C. The RT reaction buffer was prepared fresh on a daily basis and consisted of 125 μl 1.0 M EGTA, 125 μl dH₂O, 125 μl 20% Triton X100, 50 μl 1.0 M Tris (pH 7.4), 50 μl 1.0 M DTT, and 40 μl 1.0 M MgCl₂. The final reaction mixture was prepared by combining 1 part ³H-TTP, 4 parts dH₂O, 2.5 parts poly rA:oligo dT stock and 2.5 parts reaction buffer. Ten microliters of this reaction mixture was placed in a round bottom microtiter plate and 15 μl of virus-containing supernatant was added and mixed. The plate was incubated at 37° C. for 60 minutes. Following incubation, the reaction volume was spotted onto DE81 filter-mats (Wallac), washed 5 times for 5 minutes each in a 5% sodium phosphate buffer or 2×SSC (Life Technologies), 2 times for 1 minute each in distilled water, 2 times for 1 minute each in 70% ethanol, and then dried. Incorporated radioactivity (counts per minute, CPM) was quantified using standard liquid scintillation techniques.

c. MTS Staining for PBMC Viability to Measure Cytotoxicity

At assay termination, the assay plates were stained with the soluble tetrazolium-based dye MTS (CellTiter Reagent, Promega) to determine cell viability and quantify compound cytotoxicity. MTS is metabolized by the mitochondrial enzymes of metabolically active cells to yield a soluble formazan product, allowing the rapid quantitative analysis cell viability and compound cytotoxicity. The MTS is a stable solution that does not require preparation before use. At termination of the assay, 20 μl of MTS reagent was added per well. The wells were incubated for 4 hrs at 37° C. for the HIV PBMC assay. The incubation intervals were chosen based on empirically determined times for optimal dye reduction in each cell type. Adhesive plate sealers were used in place of the lids, the sealed plate was inverted several times to mix the soluble formazan product and the plate was read spectrophotometrically at 490/650 nm with a Molecular Devices Vmax plate reader.

d. Data Analysis

Using an in-house computer program, IC₅₀ (50% inhibition of virus replication), IC₉₀ (90% inhibition of virus replication), TC₅₀ (50% cytotoxicity), TC₉₀ (90% cytotoxicity), and a therapeutic index (TI=TC₅₀/IC₅₀) were calculated. Raw data for both antiviral activity and cytotoxicity with a graphic representation of the data are provided in a printout summarizing the individual compound activity. AZT was evaluated in parallel as a relevant positive control compound in the anti-HIV assay.

e. Results

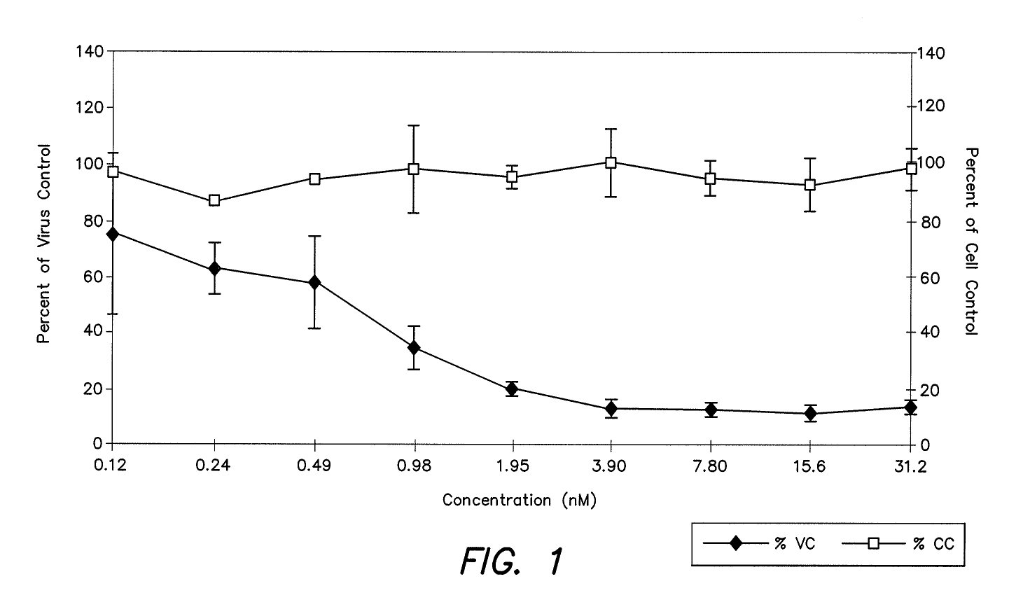

FIG. 1 shows the inhibition of HIV-1_{IIIB} replication in PBMC by native C34 (see curve ♦). This compound did not display any significant cytotoxic affect on the PBMCs as illustrated below (see curve □).

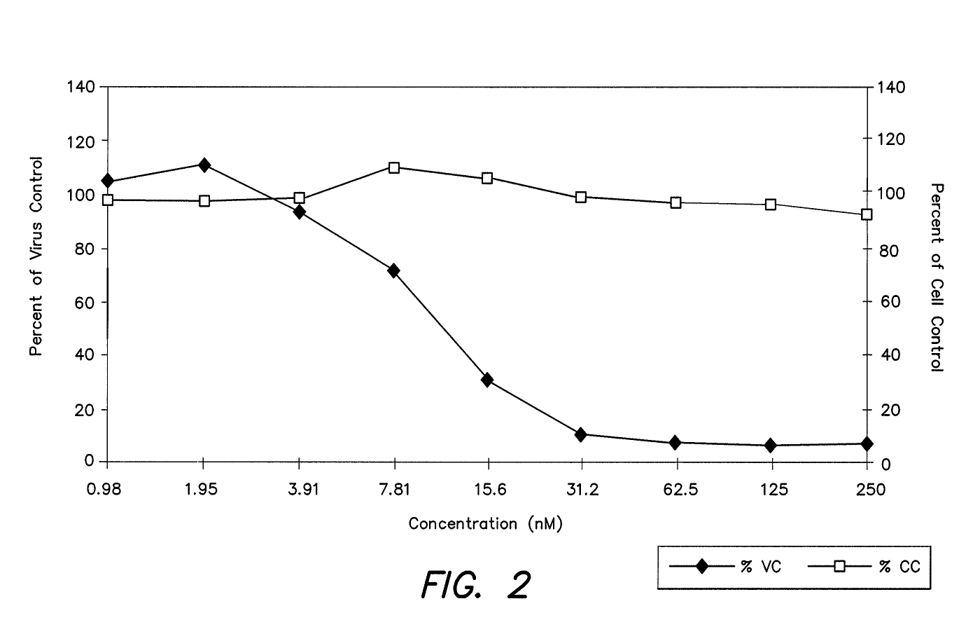

FIG. 2 shows the inhibition of HIV-1_{IIIB} replication in PBMC by the albumin conjugate of C34 having AEEA-MPA on epsilon NH₂ of lysine added at the C-terminal end, i.e. C34-Lys³ (ε-AEEA-MPA):HSA (see curve ♦). This compound did not display any cytotoxic affect on the PBMCs as illustrated below (see curve □).

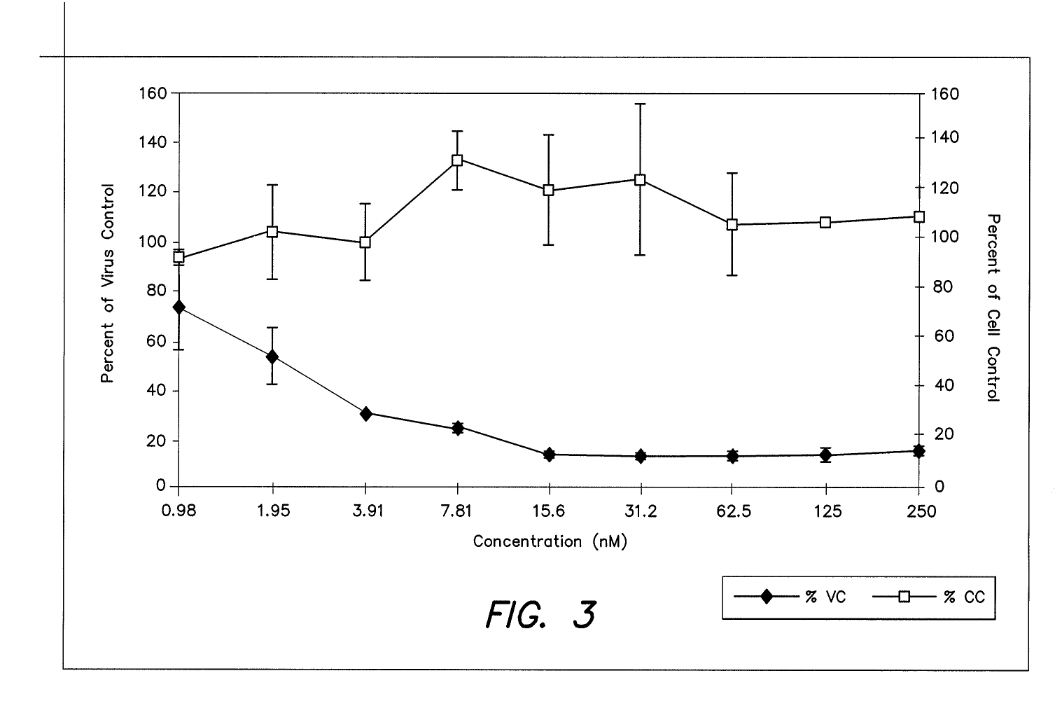

FIG. 3 shows the inhibition of HIV-1_{IIIB} replication in PBMC by the albumin conjugate of C34 having a cysteic acid at the N-terminal end, and AEEA-MPA on epsilon NH₂ of lysine added at the C-terminal end, i.e. CA-C34-Lys³⁵ (ε-AEEA-MPA):HSA (see curve ♦). This compound did not display any cytotoxic affect on the PBMCs as illustrated below (see curve □).

Based on the data illustrated in FIGS. 1, 2 and 3, the IC50 values of both albumin conjugates are given in Table 4 in comparison to that for native C34.

TABLE 4

| C34 and Albumin Conjugates | IC50 (nM) |
|---|---|
| C34 | 0.6-1.7 |
| C34-Lys³⁵(ε-AEEA-MPA):HSA | 11.2-18.9 |
| CA-C34-Lys³⁵(ε-AEEA-MPA):HSA | 1.7-2.2 |

Table 4 shows similar anti-HIV activities for native C34, albumin conjugate of C34 and albumin conjugate of CA-C34. In conclusion, addition of a cysteic acid in N-terminal and its subsequent conjugation to albumin via Lys³⁵ (ε-AEEA-MPA) does not negatively impact the activity of C34 in this assay.

### Example 8

### Additional Anti-Fusogenic Peptide Derivatives

### Experimental Procedures

The following procedures were used throughout the experiments performed to obtain the results discussed in detail below.

Synthesis of the CHR peptide analogs were performed using an automated solid-phase procedure on a Symphony Peptide Synthesizer with manual intervention during the generation of the peptides. The synthesis was performed on Fmoc-protected Ramage amide linker resin, using Fmoc-protected amino acids. Coupling was achieved by using O-benzotriazol-1-yl-N,N,N′,N′-tetramethyl-uronium hexafluorophosphate (HBTU) and diisopropylethylamine (DIEA) as the activator cocktail in N,N-dimethylformamide (DMF) solution. The Fmoc protective group was removed using 20% piperidine/DMF. A Boc-protected amino acid was used at the N-terminus in order to generate the free α-N-terminus following cleavage of the peptides from the resin. Sigmacoated glass reaction vessels were used during the synthesis.

When the maleimido is positioned at the C-terminus portion of the molecule (Table 5, albumin-conjugated Compound VII and acetylated-conjugated Compound X), the solid-phase synthesis of the peptide was initiated by the addition of Fmoc-Lys(Aloc). Aloc is a specific orthogonal protective group stable to acidic medium. The peptide chain was then elongated on solid support via the sequential addition of amino acids having their side chains protected with groups labile to acidic medium. When the peptide chain was completed, the Aloc protective group on the C-terminal lysine was removed selectively using tetrakistriphenylphosphine Palladium. The Fmoc-aminoethoxy ethoxy acetic acid (AEEA) linker was then chemically coupled to the unprotected lysine. Following classical Fmoc deprotection protocols, maleimide proprionic acid (MPA) was then chemically coupled to the AEEA spacer. Finally, the acid labile protecting groups were removed from the peptide and the peptide was then cleaved from the solid support using a strong acidic cocktail. When the maleimido is positioned at the N-terminus portion of the molecule (Table 5, maleimido-Compound VIII, albumin-conjugated Compound VIII), and albumin-conjugated-MPA-AEEA-Compound VIII, the solid-phase synthesis of the peptide was initiated by the native amino-acid sequence of the fusion peptide inhibitor.

TABLE 5

| | |
|---|---|
| HSA^{a} | Human Serum Albumin |

| | |
|---|---|
| C34 | (628)WMEWDREINNYTSLIHSLIEESQNQQEKNEQELL |
| | (661)-CONH₂^{b} |
| maleimido- | MPA^{c}-AEEA^{d}-(628)WMEWDREINNYTSLIHSLIEESQ |
| Compound | NQQEKNEQELL(661)CONH₂^{b} |
| VIII | |
| albumin- | [HSA^{a}-C34^{e}]-MPA^{c}-AEEA^{d}-(628)WMEWDREINNY |
| conjugated | TSLIHSLIEESQNQQEKNEQELL(661)-CONH₂^{b} |
| Compound | |
| VIII | |
| albumin- | [HSA^{a}-Cys34^{e}]-MPA-(628)WMEWDREINNYTSLIH |
| conjugated | SLIEESQNQQEKNEQELL(661)-CONH₂^{b} |
| Compound | |
| VII | |
| albumin- | (628)WMEWDREINNYTSLIHSLIEESQNQQEKNEQELL |
| conjugated | (661)K(εN)-AEEA^{d}-MPA^{c}-[CYS34^{e}-HSA^{a}] |
| Compound | |
| VI | |
| T-20 | Ac^{f}-(638)YTSLIHSLIEESQNQQEKNEQELLELDKWA |
| | SLWNWF(673)-CONH₂^{b} |
| albumin- | [HSA^{a}-CYS34^{e}]-MPA^{c}-AEEA^{d}-(638)YTSLIHSLI |
| conjugated | EESQNQQEKNEQELLELDKWASLWNWF(673)-CONH₂^{b} |
| Compound | |
| IX | |
| albumin- | Ac^{f}-(638)YTSLIHSLIEESQNQQEKNEQELLELDKWA |
| conjugated | SLWNWF(673)K(εN)-AEEA^{d}-MPA^{c}-[CyS34^{e}- |
| Compound | HSA^{a}] |
| X | |

^{a}HSA, human serum albumin
^{b}CONH₂, carboxamide
^{c}MPA, maleimide proprionic acid
^{d}AEEA, amino ethyl ethoxy acetic acid
^{e}CyS34, cysteine-34 of albumin
^{f}Ac, acetyl

Peptide Purification

Each product was purified by preparative reverse—phase HPLC, using a Varian (Dynamax) preparative binary HPLC system. Purification of all DAC peptides were performed using a Phenomenex Luna phenyl-hexyl (10 micron, 50 mm×250 mm) column equilibrated with a water/TFA mixture (0.1% TFA in H₂O; Solvent A) and acetonitrile/TFA (0.1% TFA in CH₃CN; Solvent B). Elution was achieved at 50 mL/min by running various gradients of Solvent B over 180 min. Fractions containing peptide were detected by UV absorbance (Varian Dynamax UVD II) at 214 and 254 nm.

Fractions were collected in 25 mL aliquots. Fractions containing the desired product were identified by mass after direct injection onto LC/MS. The selected fractions were subsequently analyzed by analytical HPLC (20-60% B over 20 min; Phenomenex Luna 5 micron phenyl-hexyl, 10 mm×250 mm column, 0.5 mL/min) to identify fractions with >90% purity for pooling. The pool was then freeze-dried using liquid nitrogen and subsequently lyophilized for at least 2 days yielding a white powder.

### Preparation of Albumin Conjugates

The conjugation of maleimido-C34 and maleimido-T-20 derivatives to cysteine-34 of HSA and subsequent purification using hydrophobic interaction chromatography has recently become an efficient process. The conjugation step involves mixing each maleimido-peptide with a 25% solution of HSA (Cortex-Biochem, San Leandro, Calif.) and incubating for 30 min at 37° C. Using an ÄKTA purifier (GE Healthcare), the resulting mixtures were loaded at a flow rate of 2.5 ml/min directly onto a 50 ml column packed with butyl sepharose 4 fast flow resin (GE Healthcare) equilibrated in 20 mM sodium phosphate buffer (pH 7) composed of 5 mM sodium octanoate and 750 mM (NH₄)₂SO₄. Under these conditions, the C34-HSA conjugates adsorbed onto the hydrophobic resin whereas essentially all non-conjugated HSA eluted within the void volume of the column. Each conjugate was further purified from any free (unreacted) maleimido-C34 derivative by applying a linear gradient of decreasing (NH₄)₂SO₄ concentration (750-0 mM) over four column volumes. Each purified conjugate was then desalted and concentrated in water using 10 kDa ultracentrifugal filter devices (Amicon; Millipore, Bedford, Mass). Finally, each conjugate solution was reformulated in an isotonic buffer solution at pH 7. Mass spectrometry of each purified sample confirmed the most abundant protein product corresponded to a 1:1 covalent complex of HSA with each maleimido derivative, and reverse-phase HPLC analysis of each purified sample confirmed the removal of essentially all unbound (free) maleimido derivative. Each albumin conjugate was formulated using sterile 0.9% NaCl and T-20 (obtained from the San Francisco General Hospital pharmacy) was dissolved in sterile water for injection and adjusted to pH 7 with HCl.

### Anti-HIV Efficacy Evaluation in Fresh Human PBMCs

HIV-1 IIIB was obtained through the AIDS Research and Reference Reagent Program, Division of AIDS, NIAID, NIH courtesy of Dr. Robert C. Gallo (Popovic M E, Read-Connole E, Gallo RC (1984) T4 positive human neoplastic cell lines susceptible to and permissive for HTLV-III. Lancet ii: 1472-1473; Popovic M, Sarngadharan M G, Read E, Gallo R C (1984) Detection, isolation, and continuous production of cytopathic retroviruses (HTLV-III) from patients with AIDS and pre-AIDS. Science 224:497-500; Ratner L et al. (1985) Complete nucleotide sequence of the AIDS virus, HTLV-III. Nature 313:277-283). Fresh human peripheral blood mononuclear cells (PBMCs), seronegative for HIV and HBV, were isolated from blood of screened donors (Biological Specialty Corporation; Colmar, Pa.) using Lymphocyte Separation Medium (LSM; Cellgro® by Mediatech, Inc.; density 1.078+/−0.002 g/ml) following the manufacturer's instructions. Cells were stimulated by incubation in 4 μg/mL Phytohemagglutinin (PHA; Sigma) for 48-72 hours. Mitogenic stimulation was maintained by the addition of 20 U/mL recombinant human IL-2 (R&D Systems, Inc) to the culture medium. PHA-stimulated PBMCs from at least two donors were pooled, diluted in fresh medium and added to 96-well plates at 5×10⁴ cells/well. Cells were infected (final MOI≅0.1) in the presence of 9 different concentrations of test compounds (triplicate wells/concentration) and incubated for 7 days. To determine the level of virus inhibition, cell-free supernatant samples were collected for analysis of reverse transcriptase activity (Buckheit R W, Swanstrom R (1991) Characterization of an HIV-1 isolate displaying an apparent absence of virion-associated reverse transcriptase activity. AIDS Res Hum Retrovir 7:295-302). Following removal of supernatant samples, compound cytotoxicity was measured by the addition of 3-(4,5-dimethylthiazol-2-yl)-5-(3-carboxymethoxyphenyl)-2-(4-sulfophenyl)-2H-tetrazolium (MTS; CellTiter 96 Reagent, Promega) following the manufacturer's instructions. Using an in-house computer program, IC₅₀ (50%, inhibition of virus replication), IC₉₀ (90%, inhibition of virus replication), TC₅₀ (50% reduction in cell viability) and selectivity index (IC₅₀/TC₅₀) were determined. AZT (nucleoside reverse transcriptase inhibitor) was used as the assay control compound.

### Viruses

The following reagent was obtained through the AIDS Research and Reference Reagent Program, Division of AIDS, NIAID, NIH: pNL4-3 from Malcolm Martin. (Adachi A et al. (1986) Production of acquired immunodeficiency syndrome-associated retrovirus in human and nonhuman cells transfected with an infectious molecular clone. J Virol 59:284-291.)

NL4-3 from the AIDS Reagent Program contains an unexpected variant DIV (G36D) mutation in gp41, which confers 8-fold resistance to T-20 in vitro A T-20-sensitive NLA-3 (NL4-3G) was altered by site-directed mutagenesis to match the consensus sequence at amino acid position 36 (aspartic acid replaced by glycine) of gp41. Stocks of NL4-3G and NL4-3D (original clone) were prepared by transfection of 293T cells and collection of supernatants on days 3. Virus stocks were titrated by 50% endpoint assay in PHA-activated PBMCs with p24 detection by ELISA.

### Results

### Antiviral Activities In-Vitro Using PBMC Based Assays

The antiviral activity of each albumin conjugate was compared to the original peptide inhibitors in vitro using a PBMC-based assay against HIV-1 IIIB (Popovic M E, Read-Connole E, Gallo R C (1984) T4 positive human neoplastic cell lines susceptible to and permissive for HTLV-III. Lancet ii:1472-1473; Popovic M, Samgadharan M G, Read E, Gallo R C (1984) Detection, isolation, and continuous production of cytopathic retroviruses (HTLV-III) from patients with AIDS and pre-AIDS. Science 224:497-500; Ratner L et al. (1985) Complete nucleotide sequence of the AIDS virus, HTLV-III. Nature 313:277-283; Buckheit R W, Swanstrom R (1991) Characterization of an HIV-1 isolate displaying an apparent absence of virion-associated reverse transcriptase activity. AIDS Res Hum Retrovir 7:295-302). Interestingly, the antiviral activity of PC-1505 (albumin-conjugated Compound VIII), compound C (albumin-conjugated Compound VII), and compound D (albumin-conjugated Compound VI) were all found to be essentially equipotent to C34 peptide and T-20 in-vitro. That is, placement of the reactive maleimide group at either the N-terminus (PC-1505 (albumin-conjugated Compound VIII) and compound C (albumin-conjugated Compound VII) or C-terminus (compound D (albumin-conjugated Compound VI) of the C34 peptide followed by albumin conjugation did not alter the antiviral activity of the fusion inhibitor (Table 7). Following albumin conjugation to T-20, there was excellent retention of antiviral activity when the reactive peptide is designed such that conjugation occurs at the N-terminal end of the peptide (compound E albumin-conjugated Compound III), whereas a significant decrease in the antiviral activity for this peptide was observed when conjugation to albumin occurs at the C-terminal end of the peptide (compound F Compound X—does cpd F have an equivalent in Erikson (Compounds I-VIII).

TABLE 6

| | | | Selectivity |
|---|---|---|---|
| Compound | IC₅₀ (nM) | IC₉₀ (nM) | Index |
| HSA | NA | NA | NA |
| C34 | 0.6 | 2.8 | >255 |
| A (maleimido-1505) | NP | NP | NP |
| maleimido-Compound | | | |
| VIII | | | |
| B preformed | 1.8 | 13.5 | >81.5 |
| conjugate-Compound | | | |
| VIII (PC-1505) | | | |
| C preformed | 11.2 | 30.2 | >22.4 |
| conjugate-Compound | | | |
| VII | | | |
| T20 | 2.2 | 9.5 | >109 |
| E preformed | 10.7 | 31.7 | >23.4 |
| conjugate-Compound | | | |
| IX | | | |
| F preformed | 87.0 | >2,000 | >23.0 |
| conjugate-Compound | | | |
| X | | | |
| AZT | 2.9 | 26.9 | >346 |

NA = IC₅₀ not achieved
NP = not performed

### Pharmacokinetic Profiles of C34 Peptide, Compound VIII and rHA in Rats

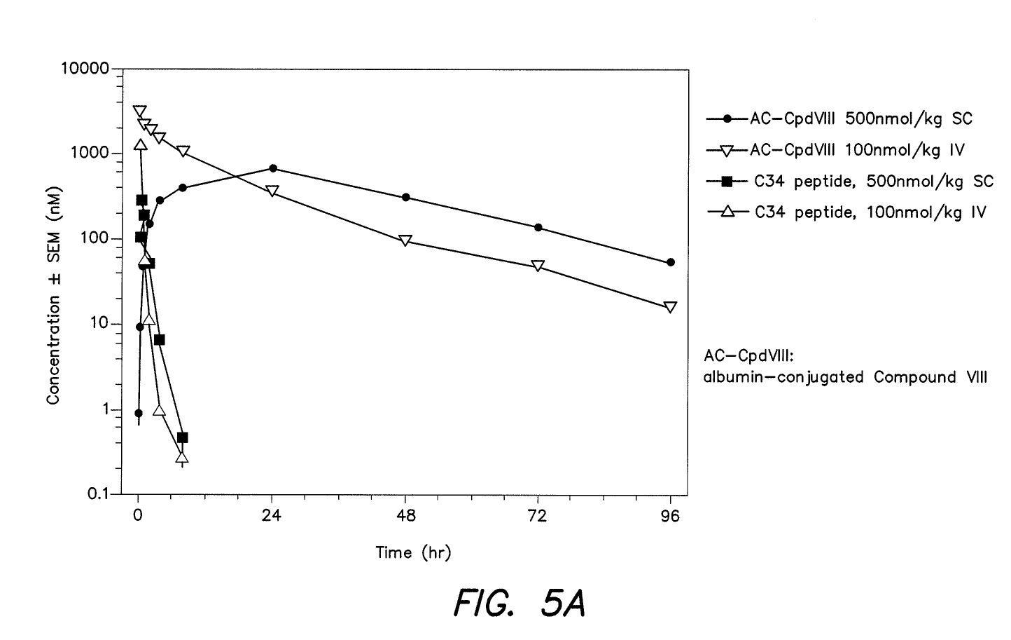

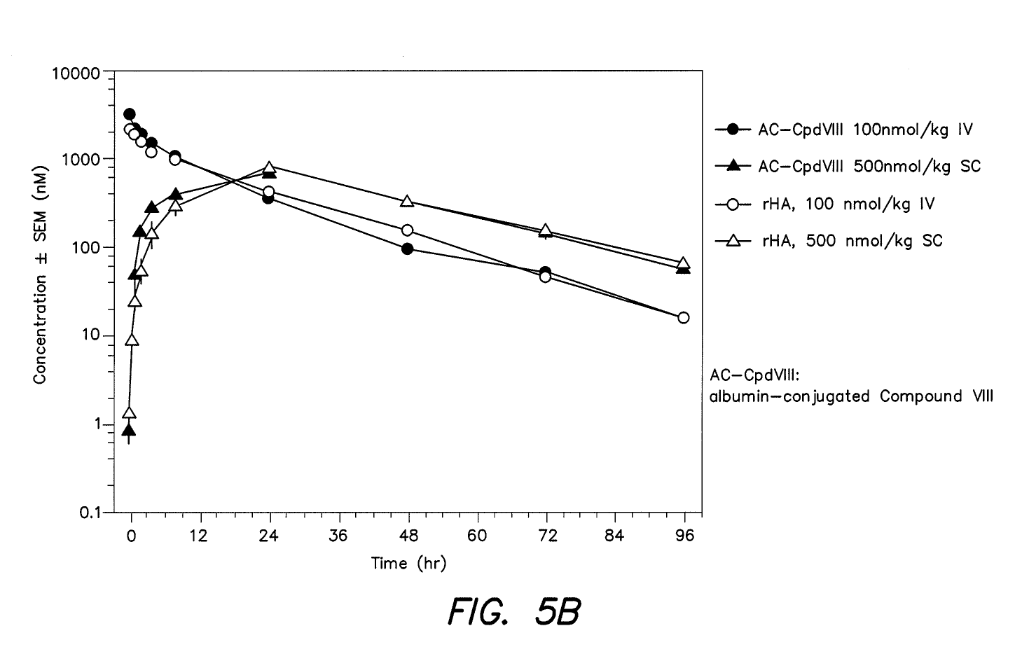

In order to ensure the antiviral activities observed in this study were due to the action of the albumin conjugates rather than to the free peptide or to the reversibility of the covalent bond between maleimide and cysteine-34, all albumin conjugates were purified to remove any unbound peptide prior to testing and the pharmacokinetic profile of Compound VIII was compared to C34 peptide (FIG. 5A) and to rHA (FIG. 5B) in rats. Clearly, exposure of C34 peptide is improved dramatically following albumin conjugation and the fact the pharmacokinetic profiles of preformed conjugate—Compound VIII is superimposed to that for rHA confirms C34 peptide has adopted a half-life closer to that of albumin. Superimposition of pharmacokinetic curves measuring for C34 peptide and HSA have also been observed using Balb/c mice for at least 30 hours following either intravenous or subcutaneous administration of preformed conjugate-Compound VIII (data not shown, T_{1/2} of albumin shorter in mice than in rats). Conversely, a slow and continuous release of C34 peptide from the conjugate would cause the two pharmacokinetic profiles to no longer superimpose as the total exposure of preformed conjugate—Compound VIII would be inferior to that of rHA. Furthermore, C34 peptide released from the conjugate would be subject to a very short half-life in vivo with limited antiviral effectiveness as compared to the long-lasting preformed conjugate-Compound VIII. Hence, the bond linking maleimide to cysteine-34 is highly stable in vivo and C34 peptide is rendered more stable against rapid renal clearance and against peptidase degradation. Taken together, it may be concluded the antiviral activities for all albumin conjugates in vitro and in vivo are due solely to the action of chemically stable conjugates rather than to reversibility of the maleimide-cysteine-34 bond.

Discussion

Synthetic peptides based upon the N-terminal helical region (NHR) and the C-terminal helical region (CHR) sequences of HIV gp41 have been shown to inhibit HIV entry by competing for exposed gp41 binding sites during the multi-step fusion process (Chan D C, Fass D, Berger J M, Kim P S (1997) Core structure of gp41 from the HIV envelope glycoprotein. Cell 89: 263-273; Chan D C, Chutkowski C T, Kim P S (1998) Evidence that a prominent cavity in the coiled coil of HIV type 1 gp41 is an attractive drug target. Proc Natl Acad Sci USA 95: 15613-15617). In the clinic, the most successful of these peptides is T-20 (Fuzeon® from Trimeris/Roche Applied Sciences) derived from the CHR of gp41. As compared to small molecules, the commercial utility of peptides is often limited by their high cost as well as their short half-lives and poor distribution in vivo. We sought to address these shortcomings by engineering CHR peptides (C34 and T-20) to bond covalently to cysteine-34 of human albumin as has already been done for other classes of peptides (Holmes D L et al. (2000) Site specific 1:1 opioid:albumin conjugate with in vitro activity and long in vivo duration. Bioconj Chem 11: 439-444; Léger R et al. (2003) Synthesis and in vitro analysis of atrial natriuretic peptide-albumin conjugates. Bioorg & Med Chem Lett 13: 3571-3575; Léger R et al. (2004) Kringle 5 peptide-albumin conjugates with anti-migratory activity. Bioorg & Med Chem Lett 14: 841-845; Léger R et al. (2004) Identification of CJC-1131-albumin bioconjugate as a stable and bioactive GLP-1 (7-36) analog. Bioorg & Med Chem Lett 14: 4395-4398; Jette L et al. (2005) Human growth hormone-releasing factor (hGRF)₁₋₂₉ albumin bioconjugates activate the GRF receptor on the anterior pituitary in rats: Identification of CJC-1295 as a long-lasting GRF analog. Endocrinology 146: 3052-3058; Thibaudeau K et al. (2005) Synthesis and evaluation of insulin-human serum albumin conjugates. Bioconj Chem 16: 1000-1008). That is, we postulated the CHR-peptide-HSA conjugates would experience a half-life in the body closer to that of albumin as opposed to a much shorter half-life for the original fusion inhibitor.

The results shown herein suggest that NHR of gp41 is more accessible than what had been originally believed. For example, to allow for such competitive inhibition to take place as that shown using preformed conjugate—Compound VIII, gp41 may be involved in a conformational equilibrium exposing the NHR region in the absence of target cells (i.e. in the context of a cell-free virus or infected cell), or that the pre-hairpin intermediate formed within the “entry claw” (Sougrat R et al. (2007) Electron tomography of the contact between T cells and SIV/HIV-1: Implications for viral entry. PLoS Pathogens 3: 0571-0581), is sufficiently solvent-exposed prior to the formation of the six helix bundle and subsequent lipid mixing and membrane puncturing steps. That is, with a Mw of ˜71 kDa for preformed conjugate—Compound VIII, our results suggest the molecular weight cutoff for accessing the NHR-trimer of gp41 is much greater than previously reported, i.e. <25 kDa (11). Second, the N-terminal segment of the C34 peptide, ⁶²⁸WMEW⁶³¹, represents the gp41 coiled-coil cavity binding residues postulated to be essential for C34 peptide's ability to inhibit HIV-1 entry (18,19). Therefore, in the case of either preformed conjugate—Compound VIII (composed of AEEA linker) or compound C preformed conjugate—Compound VII (absence of AEEA linker), how is it possible for the ⁶²⁸WMEW⁶³¹ segment of C34 peptide to reach the NHR of gp41, and simultaneously, be permanently bonded and positioned in close proximity to the surface of albumin? One possible explanation for the retention of antiviral activity for preformed conjugate—Compound VIII and compound C preformed conjugate—Compound VII is the fact that serum albumin is a highly flexible protein capable of being induced to adopt several conformational states (Peters T, Jr (1996) All about albumin-biochemistry, genetics, and medical applications, Copyright by Academic Press, Inc). For example, since C34 peptide is permanently attached to cysteine-34 of albumin, it is possible local conformational rearrangements within the unconstrained N-terminal domain of albumin (i.e. absence of disulfide bridges) cause partial unwinding so as to facilitate correct insertion of the fusion inhibitor onto the NHR region of gp41. Therefore, it is not known whether positioning of C34 peptide elsewhere within the albumin molecule other than on cysteine-34 will lead to similar conservation of antiviral activity for this fusion inhibitor (e.g. lysine residues, N-terminal or C-terminal ends), or whether similar conservation of antiviral activity would be observed following permanent conjugation of C34 peptide to other abundant serum proteins of higher molecular weight such as transferrin or IgG. Hence, it is also possible the albumin molecule plays an active participatory role rather than merely serving as a protein cargo. For example, maleylated-, aconitylated-, and succinylated-albumin function as potent HIV-1 entry inhibitors in-vitro (35-38).

Additionally, given that 24 out of the 34 amino-acid residues found in the C34 peptide overlaps with those found in T-20, how is it possible for T-20 to be a poorer candidate for albumin conjugation following modification at the C-terminus of this peptide whereas an improved retention of antiviral activity is observed when T-20 is modified at its N-terminus? One possible explanation for this finding is the recent evidence suggesting the mechanism of HIV-1 inhibition due to T-20 is distinct from that of C34 peptide (Liu S et al. (2005) J Biol Chem 280:11259-11273; Muñoz-Barroso I, et al. (1998) J Cell Biol 140: 315-23; Kliger Y et al. (2001) J Biol Chem 276:1391-1397). For example, T-20 has also been shown to inhibit recruitment of gp41 to the plasma membrane and its subsequent oligomerization at a post-lipid mixing step, whereas C34 peptide was found to be incapable of exerting its inhibitory effect following formation of the six helix bundle (Liu S et al. (2005) J Biol Chem 80:11259-11273). That is, it has been proposed that T-20 performs such inhibitory functions following its insertion into plasma membrane and that the hydrophobic C-terminal segment of T-20, ⁶⁶⁶WASLWNWF⁶⁷³, was deemed critical for effectuating these hydrophobic interactions (Mu\ddot{n}oz-Barroso I, et al. (1998) J Cell Biol 140: 315-23; Kliger Y et al. (2001) J Biol Chem 276:1391-1397). More specifically, T-20 inhibits gp41 recruitment and oligomerization by binding to the corresponding sequence within gp41 situated in close proximity to the plasma membrane (Mu\ddot{n}oz-Barroso I, Durell S, Sakaguchi K, Appella E, Blumenthal R (1998) Dilation of the human immunodeficiency virus-1 envelope glycoprotein fusion pore revealed by the inhibitory action of a synthetic peptide from gp41. J Cell Biol 140: 315-23; Kliger Y et al. (2001) J Biol Chem 276:1391-1397). Hence, the dramatic loss in antiviral activity observed for compound F Compound X, where the ⁶⁶⁶WASLWNWF⁶⁷³ sequence is positioned directly adjacent to the albumin molecule, may be attributed to this peptide's inability to function at a post lipid-mixing step as efficiently as the unconjugated (free) T-20 peptide. Conversely, the ⁶⁶⁶WASLWNWF⁶⁷³ sequence less conformationally constrained in the design of compound E (compound IX). In summary, our results provide definitive supporting evidence for reports that have suggested that T-20 and C34 peptide do not function at the same steps of HIV-1 fusion.

The results presented herein establish a proof-of-principle for this new class of albumin-peptide conjugates for inhibition of HIV or other viruses that have adopted similar mechanisms of membrane fusion and viral entry. As compared to unconjugated (free) peptide inhibitors, albumin conjugation may lead to a significantly improved exposure to the lymphatic system representing the anatomical home of approximately 98% of total HIV-infected cells (Stebbing J, Gazzard B, Douek D C (2004) Where does HIV live? N Engl J Med 350:1872-1880). This improvement may be expected due primarily to significant steady-state lymph to plasma concentration ratios observed for serum albumin (Bent-Hansen L (1991) Whole body capillary exchange of albumin. Acta Physiol Scand Suppl 603: 5-10 (Review); Porter C J H, Charman S A (2000) Lymphatic transport of proteins after subcutaneous administration. J Pharm Sci 89: 297-310), and to the efficient lymphatic uptake, transport and permeability observed for subcutaneously injected proteins larger than 16-20 kDa (Porter CJH, Charman S A (2000) Lymphatic transport of proteins after subcutaneous administration. J Pharm Sci 89: 297-310).

Finally, due to the high content of hydrophobic residues found in C34 peptide and many other antifusogenic peptides, albumin conjugation may also help remedy the low solubility limits commonly observed for this family of peptides when they are placed in simple aqueous formulations amenable for subcutaneous delivery. For example, the solubility limit of C34 peptide was found to be no more than 1 mg/ml in aqueous buffer whereas that of PC-1505 was found to be similar to that for albumin corresponding to approximately 16 mg/ml of C34 peptide (i.e. 25% (w/v) solution=250 mg/ml of PC-1505≈16 mg/ml of C34 peptide).

In summary, conjugation of antifusogenic peptides through albumin's cysteine-34 overcomes the steric block commonly associated to the NHR trimer of gp41, and thus, offers hope for the discovery of novel, larger molecular weight molecules exhibiting potent and broadly neutralizing activity. One example of an albumin-conjugated C34 peptide HIV-1 fusion inhibitor, PC-1505, may require less frequent dosing than T-20 and is likely to be an effective agent against T-20-resistant HIV-1 in humans.

### Example 9

### Additional Anti-Fusogenic Peptide Derivatives

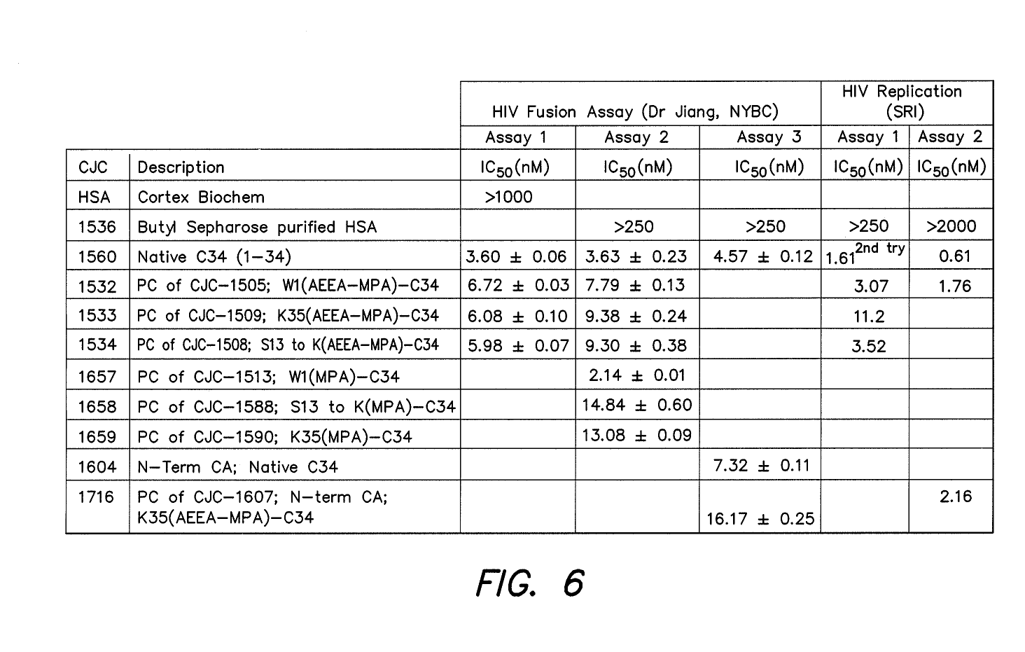

FIG. 6 depicts a table showing anti-HIV activity in vitro of several conjugates (shown as PC, preformed complexes) of the anti-fusogenic described. The assays were performed as described in the Examples herein.

While certain embodiments of the invention have been described and exemplified, those having ordinary skill in the art will understand that the invention is not intended to be limited to the specifics of any of these embodiments, but is rather defined by the accompanying claims.

We claim:

1. A modified anti-fusogenic peptide, or a conjugate thereof, wherein the peptide is modified to have increased solubility in aqueous solution at a pH ranging from about 5 to 8, compared to the peptide prior to modification, and wherein the modified peptide has the following properties:
a) shows less than about 10% precipitation in the aqueous solution at a concentration in the range of about 10 to 180 mg/ml;
b) has a solubility limit that is at least about 2.5-fold higher than the peptide prior to modification; and
c) has a solubility limit of at least about 20 mg/ml in the aqueous solution.

2. The modified anti-fusogenic peptide, or conjugate thereof, of claim 1, wherein the modified peptide comprises one or more polar moieties that are either charged or uncharged at physiological pH.

3. The modified anti-fusogenic peptide, or conjugate thereof, of claim 2, wherein the one or more polar moieties of the modified peptide comprise one or more polar or neutral side chain not found in the twenty naturally occurring amino acids.

4. The modified anti-fusogenic peptide, or conjugate thereof, of claim 3, wherein the one or more polar moieties of the modified peptide comprise one or more cysteic acids.

5. The modified anti-fusogenic peptide, or conjugate thereof, of claim 1 or 4, the one or more cysteic acids of the modified peptide are added to the N-terminal or C-terminal end of the modified anti-fusogenic peptide.

6. The modified anti-fusogenic peptide, or conjugate thereof, of claim 3, wherein the one or more polar moieties of the modified peptide do not substantially affect the secondary or tertiary structure of the peptide.

7. The modified anti-fusogenic peptide, or conjugate thereof, of claim 1 or 4, wherein the modified peptide comprises at least a portion of a gp41 coiled-coil cavity binding residues.

8. The modified anti-fusogenic peptide, or conjugate thereof, of claim 7, wherein the modified peptide comprises the amino acid sequence of C34 from amino acids ⁶²⁸WMEWDREINNYTSLIHSLIEESQNQQEKNEQELL⁶⁶¹ (SEQ ID NO:2), or up to two amino acid substitutions, insertions or deletions thereto.

9. The modified anti-fusogenic peptide, or conjugate thereof, of claim 7, wherein the modified peptide comprises the amino acid sequence of DP107 and DP178, or up to two amino acid substitutions, insertions or deletions thereto.

10. The modified anti-fusogenic peptide, or conjugate thereof, of claim 7, further comprising one or more chemically reactive moieties such that the modified peptides can react with available functionalities on blood components or carrier proteins to form stable covalent bonds of the conjugate.

11. The modified anti-fusogenic peptide, or conjugate thereof, of claim 10, wherein the reactive moiety is a maleimide-containing group.

12. The modified anti-fusogenic peptide, or conjugate thereof, of claim 10, further comprising one or more linkers selected from the group consisting of: (2-amino)ethoxy acetic acid (AEA), [2-(2-amino)ethoxy)]ethoxy acetic acid (AEEA), ethylenediamine (EDA); one or more alkyl chains (C1-C10) such as 8-aminooctanoic acid (AOA), 8-aminopropanoic acid (APA), and 4-aminobenzoic acid (APhA).

13. The modified anti-fusogenic peptide, or conjugate thereof, of claim 10, wherein the reactive moiety, with or without linker, is added to the C-terminal of the modified peptide, and the one or more polar moieties are added to the N-terminal end of the modified anti-fusogenic peptide.

14. The modified anti-fusogenic peptide, or conjugate thereof, of claim 10, wherein the reactive moiety, with or without linker, is added to the N-terminal of the modified peptide, and the one or more polar moieties are added to the C-terminal end of the modified anti-fusogenic peptide.

15. The modified anti-fusogenic peptide, or conjugate thereof, of claim 10, wherein the blood component or carrier protein is albumin.

16. The modified anti-fusogenic peptide, or conjugate thereof, of claim 15, wherein the albumin is recombinant.

17. The modified anti-fusogenic peptide, or conjugate thereof, of claim 16, wherein the albumin is covalently linked.

18. A modified anti-fusogenic peptide, or a conjugate thereof, having a configuration as follows:
[(cysteic acid)−MODIFIED PEPTIDE−Linkerₙ−Reactive Group]; or
[Reactive Group−Linkerₙ−MODIFIED PEPTIDE−(cysteic acid)].
wherein the reactive group is a maleimide-containing group covalently coupled to human serum albumin, with or without a linker;
n can be 0, 1, 2, 3, 4 or more linkers selected from the group consitisting of (2-amino)ethoxy acetic acid (AEA), [2-(2-amino)ethoxy)]ethoxy acetic acid (AEEA), ethylenediamine (EDA); one or more alkyl chains (C1-C10) such as 8-aminooctanoic acid (AOA), 8-aminopropanoic acid (APA), and 4-aminobenzoic acid (APhA); and
the modified peptide comprises the amino acid sequence of C34 from amino acids ⁶²⁸WMEWDREINNYTSLIHSLIEESQNQQEKNEQELL⁶⁶¹ (SEQ ID NO:2), or up to two amino acid substitutions or additions thereto.

19. A modified anti-fusogenic peptide, or a conjugate thereof, having a formula as follows:
(R₁)ₘ-X-(R₂)ₙ (I)
wherein in formula (I), the sum of m and n is at least 1 and m and n are each integers that are zero or greater;
X comprises the amino acid sequence of C34, DP107, DP178, or an analog thereof,
R₁ is present and R₂ is absent, R₁ is present at the N-terminus of the X group; and
When R₁ is absent and R₂ is present, R₂ is present at the C-terminus of the X group.

20. The modified anti-fusogenic peptide, or conjugate thereof, of claim 19, wherein R₁ and R₂ are each independently selected from a compound having formula (II): 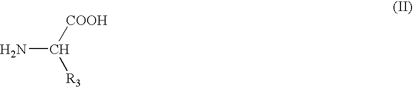
wherein the core structure of formula (II) is similar to that of an amino acid and includes an amino group, an alpha carbon and a carboxyl group; and
wherein the R₃ group of formula (II) comprises a sulfonyl group (HS═(O)₂), a sulfoxide group (HS═O), a sulfonic acid group (HO—S═(O)₂), a haloalkyl group, a secondary amine, a tertiary amine, a hydroxyl group, or other side chain group that is polar or even neutral and that can increase the overall solubility of the peptide derivative in an aqueous solution.

21. The modified anti-fusogenic peptide, or conjugate thereof, of claim 20, wherein the R₁ and R₂ groups do not substantially affect the overall secondary or the tertiary structure of the peptide. By not substantially affecting the secondary structure of the peptide conjugate, the overall activity of the peptide conjugate should not be appreciably less than that of the non-derivatized peptide.

22. A modified anti-fusogenic peptide having the structure selected from the group consisting of:
CA Compound I: (Cysteic Acid (CA) directly linked to C34; also referred to herein as
CA-C34 (SEQ ID NO:3). 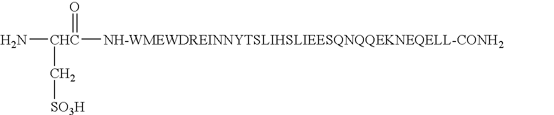
CA Compound II: (Cysteic Acid (CA) directly linked to C34 having a substitution of native Lysine at position 28 (Lys²⁸) for an arginine; also referred to herein as CA-C34 (Arg²⁸) (SEQ ID NO:4). 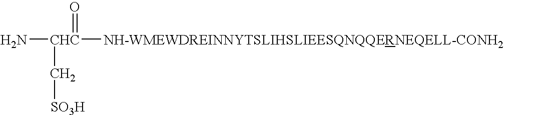
CA Compound III: (Cysteic Acid (CA) directly linked to C34 having an additional Lysine residue at position 35 (Lys³⁵), wherein the epsilon NH₂ group of lysine is coupled to the reactive group via linker (AEEA-MPA); also referred to herein as CA-C34-Lys³⁵ (ε-AEEA-MPA) (SEQ ID NO:5). 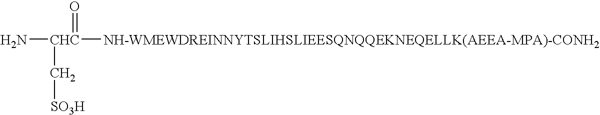
and
CA Compound IV: (Cysteic Acid (CA) directly linked to C34 having a substitution of native Lysine at position 28 (Lys²⁸) for an arginine; an additional Lysine residue at position 35 (Lys³⁵), wherein the epsilon NH₂ group of lysine is coupled to the reactive group via linker (AEEA-MPA); also referred to herein as CA-C34 (Arg²⁸)-Lys³⁵ (ε-AEEA-MPA) (SEQ ID NO:6).

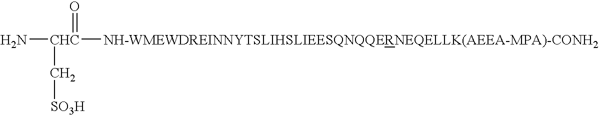

23. A conjugate comprising the modified anti-fusogenic peptide of any of claims 1, 4, 18, 19, or 22.

24. A conjugate comprising the modified anti-fusogenic peptide of claim 8 coupled, with or without a linker, to one or more amino groups, hydroxyl groups, or thiol groups on albumin to form stable covalent bonds.

25. A conjugate comprising the modified anti-fusogenic peptide of claim 10 coupled, with or without a linker, to one or more amino groups, hydroxyl groups, or thiol groups on albumin to form stable covalent bonds.

26. A conjugate comprising the modified anti-fusogenic peptide of claim 11 coupled, with or without a linker, to one or more amino groups, hydroxyl groups, or thiol groups on albumin to form stable covalent bonds.

27. A conjugate comprising the modified anti-fusogenic peptide of claim 12 coupled, with or without a linker, to one or more amino groups, hydroxyl groups, or thiol groups on albumin to form stable covalent bonds.

28. A conjugate comprising the modified anti-fusogenic peptide of claim 13 coupled, with or without a linker, to one or more amino groups, hydroxyl groups, or thiol groups on albumin to form stable covalent bonds.

29. A conjugate comprising the modified anti-fusogenic peptide of claim 15 coupled, with or without a linker, to one or more amino groups, hydroxyl groups, or thiol groups on albumin to form stable covalent bonds.

30. A conjugate comprising the modified anti-fusogenic peptide of claim 22 coupled, with or without a linker, to one or more amino groups, hydroxyl groups, or thiol groups on albumin to form stable covalent bonds.

31. A pharmaceutical composition comprising the modified anti-fusogenic peptide, or conjugate thereof, of claim 8 and a pharmaceutically acceptable carrier suitable for subcutaneous, intravenous or pulmonary administration.

32. A pharmaceutical composition comprising the modified anti-fusogenic peptide, or conjugate thereof, of claim 22 and a pharmaceutically acceptable carrier suitable for subcutaneous, intravenous or pulmonary administration.

33. A pharmaceutical composition comprising the conjugate of claim 23 and a pharmaceutically acceptable carrier suitable for subcutaneous, intravenous or pulmonary administration.

34. A pharmaceutical composition comprising the conjugate of claim 24 and a pharmaceutically acceptable carrier suitable for subcutaneous, intravenous or pulmonary administration.

35. A method of treating or preventing a virus selected from the group consisting of human immunodeficiency virus (HIV) infection, respiratory syncytial virus (RSV), human parainfluenza virus type 3 (HPIV-3), measles virus (MeV) and simian immunodeficiency virus (SIV) in a subject, comprising
administering the modified anti-fusogenic peptide, or conjugate thereof, of claim 8 to the subject having, or at risk of having, the virus, thereby treating or preventing the infection

36. A method of treating or preventing a virus selected from the group consisting of human immunodeficiency virus (HIV) infection, respiratory syncytial virus (RSV), human parainfluenza virus type 3 (HPIV-3), measles virus (MeV) and simian immunodeficiency virus (SIV) in a subject, comprising
administering the modified anti-fusogenic peptide, or conjugate thereof, of claim 22 to the subject having, or at risk of having, the virus, thereby treating or preventing the infection

37. A method of treating or preventing a virus selected from the group consisting of human immunodeficiency virus (HIV) infection, respiratory syncytial virus (RSV), human parainfluenza virus type 3 (HPIV-3), measles virus (MeV) and simian immunodeficiency virus (SIV) in a subject, comprising
administering the conjugate of claim 23 to the subject having, or at risk of having, the virus, thereby treating or preventing the infection

38. A method of treating or preventing a virus selected from the group consisting of human immunodeficiency virus (HIV) infection, respiratory syncytial virus (RSV), human parainfluenza virus type 3 (HPIV-3), measles virus (MeV) and simian immunodeficiency virus (SIV) in a subject, comprising
administering conjugate of claim 24 to a subject having, or at risk of having, the virus, thereby treating or preventing the infection

39. A methods for inhibiting one or more activities of human immunodeficiency virus (HIV) infection, respiratory syncytial virus (RSV), human parainfluenza virus type 3 (HPIV-3), measles virus (MeV) and simian immunodeficiency virus (SIV) in a subject, comprising administering to the subject in need to treatment an effective amount of the modified anti-fusogenic peptide, or conjugate thereof, of claim 8.

40. A method for inhibiting one or more activities of human immunodeficiency virus (HIV) infection, respiratory syncytial virus (RSV), human parainfluenza virus type 3 (HPIV-3), measles virus and simian immunodeficiency virus (SIV) in a subject, comprising administering to the subject in need to treatment an effective amount of the modified anti-fusogenic peptide, or conjugate thereof, of claim 22.

41. A method for enhancing the large-scale preparation of an anti-fusogenic peptide, comprising:
providing a modified anti-fusogenic peptide of claim 8; and
preparing a solution of the modified peptide that has a concentration of the modified peptide of at least 100 mg/ml.
